SEQUENCE LISTING (75 sequences)

SEQ ID NO: 1 - protein, 5 aa, Mus musculus
GYNMN

SEQ ID NO: 2 - protein, 17 aa, Mus musculus
KSSQSLLNSGNQKNYLT

SEQ ID NO: 3 - protein, 17 aa, Mus musculus
NIDPYYGATRYNQKFKG

SEQ ID NO: 4 - protein, 7 aa, Mus musculus
WASTRES

SEQ ID NO: 5 - protein, 9 aa, Mus musculus
SYYGNAFDY

SEQ ID NO: 6 - protein, 9 aa, Mus musculus
QNDYSFPFT

SEQ ID NO: 7 - protein, 17 aa, Mus musculus
YIDPYYGGTRYNQKFKG

SEQ ID NO: 8 - protein, 9 aa, Mus musculus
QNDYSYPFT

SEQ ID NO: 9 - protein, 17 aa, Mus musculus
HIDPYYVTTTYNQKFRG

SEQ ID NO: 10 - protein, 17 aa, Mus musculus
KSSQSLFNSGNQKNYLT

SEQ ID NO: 11 - protein, 9 aa, Mus musculus
SFYGNAFDY

SEQ ID NO: 12 - protein, 9 aa, Mus musculus
QNDYYYPLT

SEQ ID NO: 13 - protein, 7 aa, Mus musculus
TYFIGVG

SEQ ID NO: 14 - protein, 17 aa, Mus musculus
KSSQSLLNSGNLKNYLT

SEQ ID NO: 15 - protein, 16 aa, Mus musculus
HIWWNDNKYYNTALKS

SEQ ID NO: 16 - protein, 7 aa, Mus musculus
WASTRKS

SEQ ID NO: 17 - protein, 9 aa, Mus musculus
MGSGAWFTY

SEQ ID NO: 18 - protein, 9 aa, Mus musculus
QNDYSYPLT

SEQ ID NO: 19 - protein, 17 aa, Mus musculus
NIDPYYGGTSYNQKFKG

SEQ ID NO: 20 - protein, 17 aa, Mus musculus
KSSQNLLNNGNQKNYLT

SEQ ID NO: 21 - protein, 9 aa, Mus musculus
MYHGNAFDY

SEQ ID NO: 22 - protein, 17 aa, Mus musculus
NIDPYYGGTRYNQKFKG

SEQ ID NO: 23 - protein, 98 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 24 - protein, 95 aa, Homo sapiens
DIVMTQSPDSLAVSLGERATINCKSSQNNKNYLAWYQQKPGQPPKLLIYWASTRESGVPDRFSGSGSGTDFTLTISSLQAEDVAVYYCQQYYSTP

SEQ ID NO: 25 - protein, 118 aa, Homo sapiens
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 26 - protein, 113 aa, Homo sapiens
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIK

SEQ ID NO: 27 - protein, 118 aa, Homo sapiens
QVQLVQSGAEVKKPGASVKVSCKASGYSFTGYNMNWVRQAPGQGLEWMGNIDPYYGGTSYNQKFKGRVTLTIDKSTSTVYMELSSLRSEDTAVYYCARMYHGNAFDYWGQGTTVTVSS

SEQ ID NO: 28 - protein, 113 aa, Homo sapiens
DIVMTQSPDSLAVSLGERATMNCKSSQSLLNSGNLKNYLTWYQQKPGQPPKLLIYWASTRKSGVPDRFTGSGSGTDFTLTISSLQAEDVAVYYCQNDYSYPLTFGGGTKVEIK

SEQ ID NO: 29 - protein, 118 aa, Homo sapiens
QVQLVQSGAEVKKPGASVKVSCKASGYSFTGYNMNWVKQAPGQGLEWIGNIDPYYGGTSYNQKFKGRVTLTIDKSTSTVYMELSSLRSEDTAVYYCARMYHGNAFDYWGQGTTVTVSS

SEQ ID NO: 30 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 31 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 32 - protein, 17 aa, Artificial Sequence
  synthesic
  misc_feature (1)..(1): Xaa can be Asn, Tyr or His
  misc_feature (7)..(7): Xaa can be Gly or Val.
  misc_feature (8)..(8): Xaa can be Ala, Gly or Thr.
  misc_feature (10)..(10): Xaa can be Arg, Thr or Ser.
  misc_feature (16)..(16): Xaa can be Lys or Arg.
XIDPYYXXTXYNQKFXG

SEQ ID NO: 33 - protein, 9 aa, Artificial Sequence
  synthesic
  misc_feature (1)..(1): Xaa can be Ser or Met.
  misc_feature (2)..(2): Xaa can be Tyr or Phe.
  misc_feature (3)..(3): Xaa can be Tyr or His.
XXXGNAFDY

SEQ ID NO: 34 - protein, 17 aa, Artificial Sequence
  synthesic
  misc_feature (5)..(5): Xaa can be Ser or Asn.
  misc_feature (7)..(7): Xaa can be Leu or Phe.
  misc_feature (9)..(9): Xaa can be Ser or Asn.
  misc_feature (12)..(12): Xaa can be Gln or Leu.
KSSQXLXNXGNXKNYLT

SEQ ID NO: 35 - protein, 7 aa, Artificial Sequence
  synthesic
  misc_feature (6)..(6): Xaa can be Glu or Lys.
WASTRXS

SEQ ID NO: 36 - protein, 9 aa, Artificial Sequence
  synthesic
  misc_faeture (5)..(5): Xaa can be Ser or Tyr.
  misc_faeture (6)..(6): Xaa can be Phe or Tyr.
  misc_faeture (8)..(8): Xaa can be Phe or Leu.
QNDYXXPXT

SEQ ID NO: 37 - protein, 118 aa, Mus musculus
EFQLQQSGPELEKPGASVRISCKTSGYSFTGYNMNWVKQSNGESLEWIGNIDPYYGATRYNQKFKGKATLTVDKSSSTAYMQLKSLTSEDSAVYYCARSYYGNAFDYWGQGTTLTVSS

SEQ ID NO: 38 - protein, 113 aa, Mus musculus
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSFPFTFGSGTKLEIK

SEQ ID NO: 39 - protein, 118 aa, Mus musculus
EFQLQQSGPELEKPGASVRISCKTSGYSFTGYNMNWVKQSNGESLEWIGYIDPYYGGTRYNQKFKGKATLTVDKSSSTAYMQLKSLTSEDSAVYYCARSYYGNAFDYWGQGTTLTVSS

SEQ ID NO: 40 - protein, 113 aa, Mus musculus
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 41 - protein, 118 aa, Mus musculus
EFQLQQSGPELEKPGASVKISCKTSGYSFTGYNMNWVKQSNGQSLEWIGHIDPYYVTTTYNQKFRGKATLTVDKSSSTAYMQLKSLTSEDSAVYYCARSFYGNAFDYWGQGTTLTVSS

SEQ ID NO: 42 - protein, 113 aa, Mus musculus
DIVMTQSPSSLTVTAGEKVTMSCKSSQSLFNSGNQKNYLTWYQQKPGQPPKLLIYWASTRESGVPDRFTGSGSGTDFTLTISSVQAEDLAVYYCQNDYSFPFTFGSGTKLEIK

SEQ ID NO: 43 - protein, 119 aa, Mus musculus
QITQKESGPGILQPSQTLSLTCSLSGFSLSTYFIGVGWIRQPSGKGLEWLAHIWWNDNKYYNTALKSRLTISKDTSNNQVFLKIASVDTADTATYYCARMGSGAWFTYWGQGTLVTVSA

SEQ ID NO: 44 - protein, 113 aa, Mus musculus
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYYYPLTFGSGTKLEIK

SEQ ID NO: 45 - protein, 118 aa, Mus musculus
EFQLQQSGPELEKPGASVRISCKTSGYSFTGYNMNWVKQSNGESLEWIGNIDPYYGGTSYNQKFKGKATLTVDKSSSTAYMQLKSLTSEDSAVYYCARMYHGNAFDYWGQGTTLTVSS

SEQ ID NO: 46 - protein, 113 aa, Mus musculus
DIVMTQSPSSLTVTAGEKVTMSCKSSQSLLNSGNLKNYLTWYQQKPGQPPKLLIYWASTRKSGVPDRFTGSGSGTDFTLTLSSVQAEDLAVYYCQNDYSYPLTFGAGTKLELK

SEQ ID NO: 47 - protein, 118 aa, Mus musculus
EFQLQQSGPELEKPGASVRISCKTSGYSFTGYNMNWVKQSNGESLEWIGNIDPYYGGTRYNQKFKGKATLTVDKSSSTAYMQLKSLTSEDSAVYYCARSYYGNAFDYWGQGTTLTVSS

SEQ ID NO: 48 - protein, 113 aa, Mus musculus
DIVMTQSPSSLTVTAGEKVTMSCKSSQNLLNNGNQKNYLTWYQQKPGQPPKLLIYWASTRESGVPDRFTGSGSGTDFILTISSVQAEDLAVYYCQNDYSFPFTFGAGTKLELK

SEQ ID NO: 49 - protein, 330 aa, Homo sapiens [sequence omitted]

SEQ ID NO: 50 - protein, 107 aa, Homo sapiens [sequence omitted]

SEQ ID NO: 51 - protein, 330 aa, Artificial Sequence
  synthesic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 52 - protein, 486 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYVMASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 53 - protein, 486 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYVMASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 54 - protein, 30 aa, Artificial Sequence
  synthesic
  misc_feature (28)..(28): Xaa can be Thr or Ser.
QVQLVQSGAEVKKPGASVKVSCKASGYXFT

SEQ ID NO: 55 - protein, 14 aa, Artificial Sequence
  synthesic
  misc_faeture (3)..(3): Xaa can be Arg or Lys.
  misc_faeture (13)..(13): Xaa can be Met or IIe.
WVXQAPGQGLEWXG

SEQ ID NO: 56 - protein, 32 aa, Artificial Sequence
  synthesic
  misc_feature (4)..(4): Xaa can be Met or Leu.
RVTXTIDKSTSTVYMELSSLRSEDTAVYYCAR

SEQ ID NO: 57 - protein, 11 aa, Homo sapiens
WGQGTTVTVSS

SEQ ID NO: 58 - protein, 23 aa, Artificial Sequence
  synthesic
  misc_feature (21)..(21): Xaa can be Ile or Met.
DIVMTQSPDSLAVSLGERATXNC

SEQ ID NO: 59 - protein, 15 aa, Homo sapiens
WYQQKPGQPPKLLIY

SEQ ID NO: 60 - protein, 32 aa, Artificial Sequence
  synthesic
  misc_feature (7)..(7): Xaa can be Ser or Thr.
GVPDRFXGSGSGTDFTLTISSLQAEDVAVYYC

SEQ ID NO: 61 - protein, 10 aa, Homo sapiens
FGGGTKVEIK

SEQ ID NO: 62 - protein, 30 aa, Homo sapiens
QVQLVQSGAEVKKPGASVKVSCKASGYTFT

SEQ ID NO: 63 - protein, 30 aa, Homo sapiens
QVQLVQSGAEVKKPGASVKVSCKASGYSFT

SEQ ID NO: 64 - protein, 14 aa, Homo sapiens
WVRQAPGQGLEWMG

SEQ ID NO: 65 - protein, 14 aa, Homo sapiens
WVKQAPGQGLEWIG

SEQ ID NO: 66 - protein, 32 aa, Homo sapiens
RVTMTIDKSTSTVYMELSSLRSEDTAVYYCAR

SEQ ID NO: 67 - protein, 32 aa, Homo sapiens
RVTLTIDKSTSTVYMELSSLRSEDTAVYYCAR

SEQ ID NO: 68 - protein, 23 aa, Homo sapiens
DIVMTQSPDSLAVSLGERATINC

SEQ ID NO: 69 - protein, 23 aa, Homo sapiens
DIVMTQSPDSLAVSLGERATMNC

SEQ ID NO: 70 - protein, 32 aa, Homo sapiens
GVPDRFSGSGSGTDFTLTISSLQAEDVAVYYC

SEQ ID NO: 71 - protein, 32 aa, Homo sapiens; identical to SEQ ID NO: 70

SEQ ID NO: 72 - protein, 118 aa, Artificial Sequence
  synthesic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 73 - protein, 113 aa, Artificial Sequence; identical to SEQ ID NO: 40
  synthesic

SEQ ID NO: 74 - protein, 115 aa, Mus musculus
QIQLVQSGPELKKFGETVKISCKASGYTFTDYSIHWVKQAPGKGLKWMGWINTETGVPTYADDFKGRFAFSLETSASTAYLQINNLKNEDTATYFCARRTGFDYWGQGTTLTVSS

SEQ ID NO: 75 - protein, 112 aa, Mus musculus
DIVMTQAAFSIPVTLGTSASISCRSSKNLLHSDGITYLYWYLQRPGQSPQLLIYRVSNLASGVPNRFSGSESGTDFTLRISRVEAEDVGVYYCVQVLELPFTFGGGTKLEIK

(19) United States
(12) Patent Application Publication
(10) Pub. No.: US 2022/0306765 A1
(43) Pub. Date: Sep. 29, 2022

(54) NOVEL ANTI-CLDN18.2 ANTIBODIES

(71) Applicant: SUZHOU TRANSCENTA THERAPEUTICS CO., LTD., Suzhou, Jiangsu (CN)

(72) Inventors: Xueming QIAN, Suzhou, Jiangsu (CN); Zhen LI, Suzhou, Jiangsu (CN); Fei TENG, Suzhou, Jiangsu (CN); Hongjun LI, Suzhou, Jiangsu (CN); Hui CHAI, Suzhou, Jiangsu (CN); Huanhuan GUO, Suzhou, Jiangsu (CN)

(21) Appl. No.: 17/636,373

(22) PCT Filed: Aug. 20, 2020
(86) PCT No.: PCT/CN2020/110220

§ 371 Date: Feb. 18, 2022

(30) Foreign Application Priority Data
Aug. 20, 2019 (CN) PCT/CN2019/101563
Jun. 22, 2020 (CN) PCT/CN2020/097559

(51) Int. Cl. C07K 16/30; A61K 47/68; A61P 35/00; C07K 14/725; A61K 45/06

(52) U.S. Cl. CPC C07K 16/3076; A61K 47/6849; A61P 35/00; C07K 14/7051; A61K 45/06; A61K 2039/505

(57) ABSTRACT

Provided are anti-CLDN18.2 antibodies or antigen-binding fragments thereof, isolated polynucleotides encoding the same, pharmaceutical compositions comprising the same, and the uses thereof.

### FIELD OF THE INVENTION

The present disclosure generally relates to novel anti-CLDN18.2 antibodies that specifically bind to human CLDN18.2.

### BACKGROUND

The Claudin-18 (CLDN18) molecule (Genbank accession number: splice variant 1 (CLDN18A1 or CLDN18.1): NP_057453, NM_016369, and splice variant 2 (CLDN18A2 or CLDN18.2): NM_001002026, NP_001002026) is an integral transmembrane protein with a molecular weight of approximately 27.9/27.72 kD. CLDN18 proteins are located within the tight junctions of epithelia and endothelia that organize a network of interconnected strands of intramembranous particles between adjacent cells. CLDN18 and occludin are the most prominent transmembrane protein components in the tight junctions. Due to their strong intercellular adhesion properties, these tight junction proteins create a primary barrier to prevent and control the paracellular transport of solutes, and also restrict the lateral diffusion of membrane lipids and proteins to maintain cellular polarity. Therefore, they are critically involved in organizing epithelial tissue architecture.

CLDN18 is a member of the tetraspanin family and has 4 hydrophobic regions. CLDN18 displays several different conformations, which may be selectively addressed by antibodies (see Sahin U, Koslowski M, Dhaene K, et al. Claudin-18 splice variant 2 is a pan-cancer target suitable for therapeutic antibody development[J]. Clinical Cancer Research, 2008, 14(23): 7624-7634). CLDN18-Conformation-1 has all four hydrophobic regions serving as the transmembrane domains (TM), and two extracellular loops (loop1 embraced by hydrophobic region 1 and hydrophobic region 2; loop2 embraced by hydrophobic region 3 and 4) are formed, as described for the vast majority of CLDN family members. A second conformation (CLDN18-Conformation-2) implies that, as described for PMP22, the second and third hydrophobic domains do not fully cross the plasma membrane so that portion (loop D3) between the first and fourth transmembrane domains is extracellular. A third conformation (CLDN18-Conformation-3) shows a large extracellular domain with two internal hydrophobic regions embraced by the first and fourth hydrophobic regions. Because of a classical N-glycosylation site in the loop D3, the CLDN-18 topology variants CLDN18 topology-2 and CLDN18 topology-3 harbor an additional extracellular N-glycosylation site.

CLDN18 has two different splice variants, which are present in both mouse and human. The splice variants CLDN18.1 and CLDN18.2 differ in the first 21 amino acids at the N-terminus that comprises the first TM and the loop1, whereas the protein sequences in the C-terminus are identical (see Niimi T, Nagashima K, Ward J M, et al. Claudin-18, a novel downstream target gene for the T/EBP/NKX2. 1 homeodomain transcription factor, encodes lung- and stomach-specific isoforms through alternative splicing[J]. Molecular and cellular biology, 2001, 21(21): 7380-7390).

CLDN18.1 is selectively expressed on normal lung and stomach epithelia, whereas CLDN18.2 is only expressed on gastric cells. Most importantly, CLDN18.2 expression is restricted to the differentiated short-lived cells of stomach epithelium, but devoid from the gastric stem cell region. Using sensitive RT-PCR, both variants are not detectable in any other normal human organ. However, they are highly expressed in several cancer types including stomach, esophageal, pancreatic and lung tumors as well as human cancer cell lines (see Matsuda Y, Semba S, Ueda J, et al. Gastric and intestinal claudin expression at the invasive front of gastric carcinoma[J]. Cancer science, 2007, 98(7): 1014-1019).

There exists significant needs for novel anti-CLDN18.2 antibodies which can be used for treatment of diseases positive for CLDN18.2 expression, such as cancers.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides, among others, novel monoclonal anti-CLDN18.2 antibodies, nucleotide sequences encoding such, and the uses thereof.

In one aspect, the present disclosure provides an isolated antibody against human CLDN18.2 or an antigen-binding fragment thereof, capable of binding to an epitope comprising at least one, two, or three of amino acid residues at positions D28, W30, V43, N45, Y46, L49, W50, R51, R55, E56, F60, E62, Y66, L72, L76, V79 and R80 in the amino acid sequence of SEQ ID NO: 30.

In certain embodiments, the epitope comprises the amino acid residue at position E56. In certain embodiments, the epitope does not contain at least one of the following residues: A42, or N45. In certain embodiments, the epitope comprises the amino acid residue at position W30, L49, W50, R55, and E56. In certain embodiments, the epitope further comprises one or more amino acid residues: T41, N45, Y46, R51, F60, E62, and R80. In certain embodiments, the epitope further comprises one or more amino acid residues: D28, V43, N45, Y46, Y66, L72, L76, and V79.

In one aspect, the present disclosure provides an isolated antibody or an antigen-binding fragment thereof that are capable of specifically binding to human CLDN18.2 and having at least one of the following characteristics: a) binding to a cell expressing human CLDN18.2 at a Kd value of no more than 2.5 nM (or no more than 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4 nM) as measured by KinExA assay;

b) binding to a cell expressing human CLDN18.2 at an EC50 value of no more than 70 μg/ml (or no more than 65, 60, 55, 50, 45, 40, 35, 30, 25, 20, 15, 12, or 10, 9, 8, 7, 6, 5, 4, 3, 2, or 1 μg/ml) as measured by flow cytometry;

c) inducing complement dependent cytotoxicity (CDC) on a cell expressing human CLDN18.2 at an EC50 value of no more than 1 μg/ml (or no more than 0.9, 0.8, 0.7, 0.6, 0.5, 0.4, 0.3, 0.2, 0.1, 0.05, or 0.01 μg/ml) as measured by cytotoxicity assay;

d) inducing antibody-dependent cell cytotoxicity (ADCC) on a cell expressing human CLDN18.2 at an EC50 value of no more than 2 μg/ml (or no more than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 μg/ml) as measured by an ADCC reporter assay.

In certain embodiments, the cell comprises a NUGC4 cell, SNU-620 cell, SNU-601 cell, KATOIII cell, or a comparable cell thereof having a human CLDN18.2 protein expression level comparable to or no more than that of NUGC4 cell, SNU-620 cell, SNU-601 cell, or KATOIII cell.

In certain embodiments, the cell comprises a human CLDN18.2 high-expressing cell, a human CLDN18.2 medium-expressing cell, or a human CLDN18.2 low-expressing cell.

In certain embodiments, the human CLDN18.2 high-expressing cell expresses human CLDN18.2 at an intensity of at least 2+ as measured by IHC and at a level where at least 40% (e.g. at least 45%, at least 50%, at least 55%, at least 60%, at least 65%, at least 70%, at least 75%, at least 80%, at least 85%, at least 90%, at least 95%, 40-100%, 50-100%, 60-100%, 70-100%, 80-100%, 90-100%, 40-90%, 50-90%, 60-90%, 70-90%, 80-90%, 40-80%, 40-70%, 40-60%, 40-50%, 50-80%, 50-70%, 50-60%, 60-80%, 60-70%, or 70-80%) of the cells are stained positive in Immunohistochemistry (IHC); the human CLDN18.2 medium-expressing cell expresses human CLDN18.2 at an intensity of at least 1+ and below 2+ as measured by IHC and at a level where at least 30% (or at least 35%) but below 40% of the cells are stained positive in IHC; and the human CLDN18.2 low-expressing cell expresses human CLDN18.2 at an intensity of above 0 but below 1+ as measured by IHC and at a level where above 0 but below 30% (e.g. 5%, 10%, 15%, 20%, 25%, 5-25%, 10-25%, 15-25%, 20-25%, 5-20%, 5-15%, 5-10%, 10-20%, or 10-15%) of the cells are stained positive in IHC.

In certain embodiments, the EC50 value for binding to NUGC4 cells is no more than 70 μg/ml (or no more than 65, 60, 55, 50, 45, 40, 35, 30, 25, 20, 15, 12, or g/ml).

In certain embodiments, the ADCC on NUGC4 cells at an EC50 value of no more than 2 μg/ml (or no more than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 μg/ml) as measured by an ADCC reporter assay.

In one aspect, the present disclosure provides an isolated antibody or an antigen-binding fragment thereof that are capable of specifically binding to human CLDN18.2 and having at least one of the following characteristics:
a) binding to human CLDN18.2 at a Kd value no more than 80%, 70%, 60%, 50%, 40%, 30%, 20%, 15% of that of IMAB362, as measured by KinExA assay;
b) binding to a cell expressing human or mouse CLDN18.2 at an EC50 value no more than 80%, 70%, 60%, 50%, 40%, 30%, 20%, 15% or 10% of that of IMAB362, as measured by flow cytometry assay;
c) inducing complement dependent cytotoxicity (CDC) on a cell expressing human CLDN18.2 at an EC50 value no more than 80%, 70%, 60%, 50%, 40%, 30%, 20%, 10%, or 5% of that of IMAB362, as measured by cytotoxicity assay; and
d) inducing antibody-dependent cell cytotoxicity (ADCC) on a cell expressing human CLDN18.2 at an EC50 value no more than 80%, 70%, 60%, 50%, 40%, 30%, 20%, 10%, 5%, or 1% of that of IMAB362, as measured by an ADCC reporter assay,
wherein IMAB362 is an antibody comprising a heavy chain variable region comprising the amino acid sequence of SEQ ID NO: 72, and a light chain variable region comprising the amino acid sequence of SEQ ID NO: 73.

In certain embodiments, the cell comprises a NUGC4 cell, SNU-620 cell, SNU-601 cell, KATOIII cell, or a cell line having a human CLDN18.2 protein expression level comparable to or no more than that of that of NUGC4 cell, SNU-620 cell, SNU-601 cell, or KATOIII cell. In certain embodiments, the cell comprises a human CLDN18.2 high-expressing cell, a human CLDN18.2 medium-expressing cell, or a human CLDN18.2 low-expressing cell.

In certain embodiments, the human CLDN18.2 high-expressing cell expresses human CLDN18.2 at an intensity of at least 2+ as measured by IHC and at a level where at least 40% (e.g. at least 45%, at least 50%, at least 55%, at least 60%, at least 65%, at least 70%, at least 75%, at least 80%, at least 85%, at least 90%, at least 95%, 40-100%, 50-100%, 60-100%, 70-100%, 80-100%, 90-100%, 40-90%, 50-90%, 60-90%, 70-90%, 80-90%, 40-80%, 40-70%, 40-60%, 40-50%, 50-80%, 50-70%, 50-60%, 60-80%, 60-70%, or 70-80%) of the cells are stained positive in IHC; the human CLDN18.2 medium-expressing cell expresses human CLDN18.2 at an intensity of at least 1+ and below 2+ as measured by IHC and at a level where at least 30% (or at least 35%) but below 40% of the cells are stained positive in IHC; and the human CLDN18.2 low-expressing cell expresses human CLDN18.2 at an intensity of above 0 but below 1+ as measured by IHC and at a level where above 0 but below 30% (e.g. 5%, 10%, 15%, 20%, 25%, 5-25%, 10-25%, 15-25%, 20-25%, 5-20%, 5-15%, 5-10%, 10-20%, or 10-15%) of the cells are stained positive in IHC.

In certain embodiments, the isolated antibodies or the antigen-binding fragments thereof capable of binding to an epitope comprising at least one, two, or three of amino acid residues at positions D28, W30, V43, N45, Y46, L49, W50, R51, R55, E56, F60, E62, Y66, L72, L76, V79 and R80 in the amino acid sequence of SEQ ID NO: 30. In certain embodiments, the epitope comprises the amino acid residue at position E56. In certain embodiments, the epitope does not contain at least one of the following residues: A42 or N45. In certain embodiments, the epitope comprises the amino acid residue at position W30, L49, W50, R55, and E56. In certain embodiments, the epitope further comprises one or more amino acid residues: T41, N45, Y46, R51, F60, E62, and R80. In certain embodiments, the epitope further comprises one or more amino acid residues: D28, V43, N45, Y46, Y66, L72, L76, and V79.

In one aspect, the present disclosure provides an anti-CLDN18.2 antibody or an antigen-binding fragment thereof, comprising heavy chain HCDR1, HCDR2 and HCDR3 and/or light chain LCDR1, LCDR2 and LCDR3 sequences, wherein
the HCDR1 sequence comprises GYNMN (SEQ ID NO: 1), or TYFIGVG (SEQ ID NO: 13), or a homologue sequence of at least 80% sequence identity thereof;
the HCDR2 sequence comprises X₁IDPYYX₂X₃TX₄YNQKFX₅G (SEQ ID NO: 32), or HIWWNDNKYYNTALKS (SEQ ID NO: 15), or a homologue sequence of at least 80% (or at least 85%, 90%, 95%) sequence identity thereof; the HCDR3 sequence comprises X₆X₇X₈GNAFDY (SEQ ID NO: 33), or MGSGAWFTY (SEQ ID NO: 17), or a homologue sequence of at least 80% sequence identity thereof;
the LCDR1 sequence comprises KSSQX₉LX₁₀NX₁₁GNX₁₂KNYLT (SEQ ID NO: 34) or a homologue sequence of at least 80% (or at least 85%, 90%, 95%) sequence identity thereof;
the LCDR2 sequence comprises WASTRX₁₃S (SEQ ID NO: 35) or a homologue sequence of at least 80% sequence identity thereof;
the LCDR3 sequence comprises QNDYX₁₄X₁₅PX₁₆T (SEQ ID NO: 36) or a homologue sequence of at least 80% sequence identity thereof;
wherein X₁ is N or Y or H, X₂ is G or V, X₃ is A or G or T, X₄ is R or T or S, X₅ is K or R, X₆ is S or M, X₇ is Y or F, X₈ is Y or H, X₉ is S or N, X₁₀ is L or F, Xᵤ is S or N, X₁₂ is Q or L, X₁₃ is E or K, X₁₄ is S or Y, X₁₅ is F or Y and X₁₆ is F or L.

In one aspect, the present disclosure provides an anti-CLDN18.2 antibody or an antigen-binding fragment thereof provided herein, wherein the heavy chain variable region comprises:
a) a HCDR1 comprises a sequence selected from SEQ ID NO: 1, and SEQ ID NO: 13,
b) a HCDR2 comprises a sequence selected from SEQ ID NO: 3, SEQ ID NO: 7, SEQ ID NO: 9, SEQ ID NO: 15, SEQ ID NO: 19, and SEQ ID NO: 22, and
c) a HCDR3 comprises a sequence selected from SEQ ID NO: 5, SEQ ID NO: 11, SEQ ID NO: 17, and SEQ ID NO: 21, and/or the light chain variable region comprises:
d) a LCDR1 comprises a sequence of SEQ ID NO: 2, SEQ ID NO: 10, SEQ ID NO: 14, and SEQ ID NO: 20,
e) a LCDR2 comprises a sequence of SEQ ID NO: 4, and SEQ ID NO: 16, and
f) a LCDR3 comprises a sequence selected from SEQ ID NO: 6, SEQ ID NO: 8, SEQ ID NO: 12, and SEQ ID NO: 18.

In certain embodiments, the antibody or an antigen-binding fragment thereof provided herein, wherein the heavy chain variable region is selected from the group consisting of:
a) a heavy chain variable region comprising a HCDR1 comprising the sequence of SEQ ID NO: 1, a HCDR2 comprising the sequence of SEQ ID NO: 3, and a HCDR3 comprising the sequence of SEQ ID NO: 5;
b) a heavy chain variable region comprising a HCDR1 comprising the sequence of SEQ ID NO: 1, a HCDR2 comprising the sequence of SEQ ID NO: 7, and a HCDR3 comprising the sequence of SEQ ID NO: 5;
c) a heavy chain variable region comprising a HCDR1 comprising the sequence of SEQ ID NO: 1, a HCDR2 comprising the sequence of SEQ ID NO: 9, and a HCDR3 comprising the sequence of SEQ ID NO: 11;
d) a heavy chain variable region comprising a HCDR1 comprising the sequence of SEQ ID NO: 13, a HCDR2 comprising the sequence of SEQ ID NO: 15, and a HCDR3 comprising the sequence of SEQ ID NO: 17;
e) a heavy chain variable region comprising a HCDR1 comprising the sequence of SEQ ID NO: 1, a HCDR2 comprising the sequence of SEQ ID NO: 19, and a HCDR3 comprising the sequence of SEQ ID NO: 21; and
f) a heavy chain variable region comprising a HCDR1 comprising the sequence of SEQ ID NO: 1, a HCDR2 comprising the sequence of SEQ ID NO: 22, and a HCDR3 comprising the sequence of SEQ ID NO: 5.

In certain embodiments, the antibody or an antigen-binding fragment thereof provided herein, wherein the light chain variable region is selected from the group consisting of:
a) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 6;
b) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 8;
c) a light chain variable region comprising a LCDR1 comprising the sequence of SEQ ID NO: 10, a LCDR2 comprising the sequence of SEQ ID NO: 4, and a LCDR3 comprising the sequence of SEQ ID NO: 6;
d) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 12;
e) a light chain variable region comprising a LCDR1 comprising the sequence of SEQ ID NO: 14, a LCDR2 comprising the sequence of SEQ ID NO: 16, and a LCDR3 comprising the sequence of SEQ ID NO: 18; and
f) a light chain variable region comprising a LCDR1 comprising the sequence of SEQ ID NO: 20, a LCDR2 comprising the sequence of SEQ ID NO: 4, and a LCDR3 comprising the sequence of SEQ ID NO: 6.

In certain embodiments, the antibody or an antigen-binding fragment thereof provided herein, wherein:
a) the heavy chain variable region comprises a HCDR1 comprising the sequence of SEQ ID NO: 1, a HCDR2 comprising the sequence of SEQ ID NO: 3, and a HCDR3 comprising the sequence of SEQ ID NO: 5; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 6;
b) the heavy chain variable region comprises a HCDR1 comprising the sequence of SEQ ID NO: 1, a HCDR2 comprising the sequence of SEQ ID NO: 7, and a HCDR3 comprising the sequence of SEQ ID NO: 5; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 8;
c) the heavy chain variable region comprises a HCDR1 comprising the sequence of SEQ ID NO: 1, a HCDR2 comprising the sequence of SEQ ID NO: 9, and a HCDR3 comprising the sequence of SEQ ID NO: 11; and the light chain variable region comprises a LCDR1 comprising the sequence of SEQ ID NO: 10, a LCDR2 comprising the sequence of SEQ ID NO: 4, and a LCDR3 comprising the sequence of SEQ ID NO: 6;
d) the heavy chain variable region comprises a HCDR1 comprising the sequence of SEQ ID NO: 13, a HCDR2 comprising the sequence of SEQ ID NO: 15, and a HCDR3 comprising the sequence of SEQ ID NO: 17; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 12;
e) the heavy chain variable region comprises a HCDR1 comprising the sequence of SEQ ID NO: 1, a HCDR2 comprising the sequence of SEQ ID NO: 19, and a HCDR3 comprising the sequence of SEQ ID NO: 21; and the light chain variable region comprises a LCDR1 comprising the sequence of SEQ ID NO: 14, a LCDR2 comprising the sequence of SEQ ID NO: 16, and a LCDR3 comprising the sequence of SEQ ID NO: 18; or
f) the heavy chain variable region comprises a HCDR1 comprising the sequence of SEQ ID NO: 1, a HCDR2 comprising the sequence of SEQ ID NO: 22, and a HCDR3 comprising the sequence of SEQ ID NO: 5; and the light chain variable region comprises a LCDR1 comprising the sequence of SEQ ID NO: 20, a LCDR2 comprising the sequence of SEQ ID NO: 4, and a LCDR3 comprising the sequence of SEQ ID NO: 6.

In certain embodiments, wherein the heavy chain variable region comprises a sequence selected from the group consisting of SEQ ID NO: 25, SEQ ID NO: 27, SEQ ID NO: 29, SEQ ID NO: 37, SEQ ID NO: 39, SEQ ID NO: 41, SEQ ID NO: 43, SEQ ID NO: 45, and SEQ ID NO: 47, and a homologous sequence thereof having at least 80% (e.g. at least 85%, 90%, 95%, 96%, 97%, 98%, or 99%) sequence identity yet retaining specific binding affinity to CLDN18.2.

In certain embodiments, wherein the light chain variable region comprises a sequence selected from the group consisting of SEQ ID NO: 26, SEQ ID NO: 28, SEQ ID NO: 38, SEQ ID NO: 40, SEQ ID NO: 42, SEQ ID NO: 44, SEQ ID NO: 46, SEQ ID NO: 48, and a homologous sequence thereof having at least 80% (e.g. at least 85%, 90%, 95%, 96%, 97%, 98%, or 99%) sequence identity yet retaining specific binding affinity to CLDN18.2.

In certain embodiments, the antibody or an antigen-binding fragment thereof provided herein, wherein:
a) a heavy chain variable region comprising the sequence of SEQ ID NO: 23 and a light chain variable region comprising the sequence of SEQ ID NO: 24;
b) the heavy chain variable region comprises a sequence of SEQ ID NO: 25 and the light chain variable region comprises a sequence of SEQ ID NO: 26;
c) the heavy chain variable region comprises a sequence of SEQ ID NO: 27 and the light chain variable region comprises a sequence of SEQ ID NO: 28;
d) the heavy chain variable region comprises a sequence of SEQ ID NO: 29 and the light chain variable region comprises a sequence of SEQ ID NO: 26, or 28;
e) the heavy chain variable region comprises a sequence of SEQ ID NO: 37 and the light chain variable region comprises a sequence of SEQ ID NO: 38;
f) the heavy chain variable region comprises a sequence of SEQ ID NO: 39 and the light chain variable region comprises a sequence of SEQ ID NO: 40;
g) the heavy chain variable region comprises a sequence of SEQ ID NO: 41 and the light chain variable region comprises a sequence of SEQ ID NO: 42;
h) the heavy chain variable region comprises a sequence of SEQ ID NO: 43 and the light chain variable region comprises a sequence of SEQ ID NO: 44;
i) the heavy chain variable region comprises a sequence of SEQ ID NO: 45 and the light chain variable region comprises a sequence of SEQ ID NO: 46; or
j) the heavy chain variable region comprises a sequence of SEQ ID NO: 47 and the light chain variable region comprises a sequence of SEQ ID NO: 48.

In certain embodiments, the anti-CLDN18.2 antibody or an antigen-binding fragment thereof provided herein further comprises one or more of heavy chain HFR1, HFR2, HFR3 and HFR4, and/or one or more of light chain LFR1, LFR2, LFR3 and LFR4, wherein:
the HFR1 comprises QVQLVQSGAEVKKPGASVKVSCKASGYX₁₇FT (SEQ ID NO: 54) or a homologous sequence of at least 80% (or at least 85%, 90%, 95%) sequence identity thereof,
the HFR2 comprises WVX₁₈QAPGQGLEWX₁₉G (SEQ ID NO: 55) or a homologous sequence of at least 80% (or at least 90%) sequence identity thereof, the HFR3 sequence comprises
RVTX₂₀TIDKSTSTVYMELSSLRSEDTAVYYCAR (SEQ ID NO: 56) or a homologous sequence of at least 80% (or at least 85%, 90%, 95%) sequence identity thereof,
the HFR4 comprises WGQGTTVTVSS (SEQ ID NO: 57) or a homologous sequence of at least 80% sequence identity thereof,
the LFR1 comprises DIVMTQSPDSLAVSLGERATX₂₁NC (SEQ ID NO: 58) or a homologous sequence of at least 80% (or at least 85%, 90%, 95%) sequence identity thereof,
the LFR2 comprises WYQQKPGQPPKLLIY (SEQ ID NO: 59) or a homologous sequence of at least 80% (or at least 85%, 90%) sequence identity thereof,
the LFR3 comprises GVPDRFX₂₂GSGSGTDFTLTISSLQAEDVAVYYC (SEQ ID NO: 60) or a homologous sequence of at least 80% (or at least 85%, 90%, 95%) sequence identity thereof, and
the LFR4 comprises FGGGTKVEIK (SEQ ID NO: 61) or a homologous sequence of at least 80% (or at least 90%) sequence identity thereof,
wherein X₁₇ is T or S, X₁₈ is R or K, X₁₉ is M or I, X₂₀ is M or L, X₂₁ is I or M, and X₂₂ is S or T.

In certain embodiments,
the HFR1 comprises a sequence selected from the group consisting of SEQ ID NOs: 62 and 63,
the HFR2 comprises a sequence selected from the group consisting of SEQ ID NOs: 64 and 65,
the HFR3 comprises the sequence selected from the group consisting of SEQ ID NOs: 66 and 67,
the HFR4 comprises a sequence of SEQ ID NOs: 57,
the LFR1 comprises the sequence from the group consisting of SEQ ID NOs: 68 and 69,
the LFR2 comprises a sequence of SEQ ID NO: 59,
the LFR3 comprises a sequence selected from the group consisting of SEQ ID NOs: 70 and 71, and
the LFR4 comprises a sequence of SEQ ID NO: 61.

In certain embodiments, the antibody or antigen-binding fragment thereof provided herein, further comprising one or more amino acid residue substitutions or modifications yet retains specific binding affinity to CLDN18.2. In certain embodiments, at least one of the substitutions or modifications is in one or more of the CDR sequences, and/or in one or more non-CDR regions of the VH or VL sequences.

In certain embodiments, the antibody binds to an epitope comprising at least one, two, or three of amino acid residues at positions D28, W30, V43, N45, Y46, L49, W50, R51, R55, E56, F60, E62, Y66, L72, L76, V79 and R80 of human CLDN18.2 having the amino acid sequence of SEQ ID NO: 30.

In certain embodiments, the antibodies or antigen-binding fragments thereof comprising an immunoglobulin constant region, optionally a constant region of human Ig, or optionally a constant region of human IgG. In certain embodiments, the constant region comprises a constant region of human IgG1, IgG2, IgG3, or IgG4. In certain embodiments, the constant region of human IgG1 comprises SEQ ID NO: 49, or a homologous sequence having at least 80% (e.g. at least 85%, 90%, 95%, 96%, 97%, 98%, or 99%) sequence identity thereof.

In certain embodiments, the constant region comprises one or more amino acid residue substitutions or modifications conferring increased CDC or ADCC relative to wild-type constant region. In certain embodiments, the constant region comprises one or more amino acid residue substitutions relative to SEQ ID NO: 49, selected from the group consisting of: L235V, F243L, R292P, Y300L, P396L, or any combination thereof. In certain embodiments, the constant region comprises the sequence of SEQ ID NO: 51.

In certain embodiments, the antibody or antigen-binding fragment thereof is afucosylated.

In certain embodiments, the antibody or antigen-binding fragment thereof is humanized. In certain embodiments, the antibody or antigen-binding fragment thereof is a camelized single domain antibody, a diabody, a scFv, an scFv dimer, a BsFv, a dsFv, a (dsFv)₂, an Fv fragment, a Fab, a Fab′, a F(ab′)₂, a ds diabody, a nanobody, a domain antibody, or a bivalent domain antibody.

In certain embodiments, the antibody or antigen-binding fragment thereof is bispecific. In certain embodiments, the antibody or antigen-binding fragment thereof is capable of specifically binding to a first epitope on CLDN18.2, and a second epitope that is on CLDN18.2 or on a second antigen different from CLDN18.2. In certain embodiments, the second antigen is an immune related target, optionally selected from the group consisting of: PD-L1, PD-L2, PD-1, CLTA-4, TIM-3, LAG3, CD160, 2B4, TGF β, VISTA, BTLA, TIGIT, LAIR1, OX40, CD2, CD27, ICAM-1, NKG2C, SLAMF7, NKp80, CD160, B7-H3, LFA-1, 1COS, 4-1BB, GITR, CD30, CD40, BAFFR, HVEM, CD7, LIGHT, IL-2, IL-15, CD3, CD16 and CD83.

In certain embodiments, the second antigen comprises a tumor antigen. In certain embodiments, the tumor antigen is present in a CLDN18.2-expressing cell.

In embodiments, the tumor antigen comprises CA-125, gangliosides G (D2), G (M2) and G (D3), CD20, CD52, CD33, Ep-CAM, CEA, bombesin-like peptides, PSA, HER2/neu, epidermal growth factor receptor (EGFR), erbB2, erbB3/HER3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R or CO17-1A.

In certain embodiments, the antibody or an antigen-binding fragment thereof of is capable of specifically binding to mouse CLDN18.2. In certain embodiments, the antibody or an antigen-binding fragment thereof does not bind to human CLDN18.1.

In certain embodiments, the antibody or antigen-binding fragment thereof is linked to one or more conjugate moieties. In certain embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

In one aspect, the present disclosure provides an antibody or an antigen-binding fragment thereof, which competes for binding to CLDN18.2 with the antibody or antigen-binding fragment thereof provided herein.

In one aspect, the present disclosure provides a pharmaceutical composition comprising the antibody or antigen-binding fragment thereof of provided herein and one or more pharmaceutically acceptable carriers.

In one aspect, the present disclosure provides an isolated polynucleotide encoding the antibody or an antigen-binding fragment thereof provided herein. In one aspect, the present disclosure provides a vector comprising the isolated polynucleotide provided herein. In one aspect, the present disclosure provides a host cell comprising the vector provided herein.

In one aspect, the present disclosure provides methods of expressing the antibody or antigen-binding fragment thereof provided herein, comprising culturing the host cell provided herein under the condition at which the vector provided herein is expressed.

In one aspect, the present disclosure provides methods of treating a disease or condition in a subject that would benefit from modulation of CLDN18.2 activity, comprising administering to the subject a therapeutically effective amount of the antibody or antigen-binding fragment thereof provided herein and/or the pharmaceutical composition provided herein. In certain embodiments, the disease or condition is a CLDN18.2 related disease or condition. In certain embodiments, the disease or condition is cancer, optionally CLDN18.2-expressing cancer. In certain embodiments, the subject is identified as having a CLDN18.2-expressing cancer cell. In certain embodiments, the subject is identified as having a CLDN18.2 high-expressing cancer cell, a CLDN18.2 medium-expressing cancer cell, or a CLDN18.2 low-expressing cancer cell. In certain embodiments, the CLDN18.2 high-expressing cancer cell expresses CLDN18.2 at an intensity of at least 2+ as measured by IHC and at a level where at least 40% (e.g. at least 45%, at least 50%, at least 55%, at least 60%, at least 65%, at least 70%, at least 75%, at least 80%, at least 85%, at least 90%, at least 95%, 40-100%, 50-100%, 60-100%, 70-100%, 80-100%, 90-100%, 40-90%, 50-90%, 60-90%, 70-90%, 80-90%, 40-80%, 40-70%, 40-60%, 40-50%, 50-80%, 50-70%, 50-60%, 60-80%, 60-70%, or 70-80%) of the cells are stained positive in IHC; the CLDN18.2 medium-expressing cancer cell expresses CLDN18.2 at an intensity of at least 1+ and below 2+ as measured by IHC and at a level where at least 30% (or at least 35%) but below 40% of the cells are stained positive in IHC, and the CLDN18.2 low-expressing cancer cell expresses CLDN18.2 at an intensity of above 0 but below 1+ as measured by IHC and at a level where above 0 but below 30% (e.g. 5%, 10%, 15%, 20%, 25%, 5-25%, 10-25%, 15-25%, 20-25%, 5-20%, 5-15%, 5-10%, 10-20%, or 10-15%) of the cells are stained positive in IHC.

In certain embodiments, the method further comprises administering a therapeutically effective amount of a second therapy agent. In certain embodiments, the disease or condition is a CLDN18.2 related disease or condition. In certain embodiments, the disease or condition is cancer, optionally CLDN18.2-expressing cancer.

In certain embodiments, the subject is human.

In certain embodiments, the administration is via oral, nasal, intravenous, subcutaneous, sublingual, or intramuscular administration.

In certain embodiments, the methods further comprises administering a therapeutically effective amount of a second therapeutic agent. In certain embodiments, the second therapy agent is selected from a chemotherapeutic agent, an anti-cancer drug, radiation therapy, an immunotherapy agent, anti-angiogenesis agent, a targeted therapy agent, a cellular therapy agent, a gene therapy agent, a hormonal therapy agent, or cytokines.

In one aspect, the present disclosure provides a kit comprising an antibody or an antigen-binding fragment thereof provided herein and a second therapeutic agent.

In one aspect, the present disclosure provides methods of modulating CLDN18.2 activity in a CLDN18.2-expressing cell, comprising exposing the CLDN18.2-expressing cell to the antibody or antigen-binding fragment thereof provided herein.

In one aspect, the present disclosure provides methods of detecting presence or amount of CLDN18.2 in a sample, comprising contacting the sample with the antibody or antigen-binding fragment thereof provided herein, and determining the presence or the amount of CLDN18.2 in the sample.

In one aspect, the present disclosure provides methods of diagnosing a CLDN18.2 related disease or condition in a subject, comprising: a) contacting a sample obtained from the subject with the antibody or antigen-binding fragment thereof provided herein; b) determining presence or amount of CLDN18.2 in the sample; and c) correlating the presence or the amount of CLDN18.2 to existence or status of the CLDN18.2 related disease or condition in the subject.

In one aspect, the present disclosure provides use of the antibody or antigen-binding fragment thereof provided herein in the manufacture of a medicament for treating a CLDN18.2 related disease or condition in a subject.

In one aspect, the present disclosure provides use of the antibody or antigen-binding fragment thereof provided herein in the manufacture of a diagnostic reagent for diagnosing a CLDN18.2 related disease or condition.

In one aspect, the present disclosure provides a kit comprising the antibody or antigen-binding fragment thereof provided herein, which is useful in detecting CLDN18.2.

In one aspect, the present disclosure provides a chimeric antigen receptor (CAR) comprising an antigen binding domain, a transmembrane domain, a costimulatory signaling region, and a TCR signaling domain, wherein the antigen binding domain specifically binds to CLDN18.2 and comprises an antigen binding fragment thereof provided herein.

In certain embodiments, the antigen binding fragment is a Fab or a scFv.

In certain embodiments, the CAR is bispecific. In certain embodiments, the CAR is capable of specifically binding to a first epitope on CLDN18.2, and a second epitope. In certain embodiments, the second epitope is on CLDN18.2. In certain embodiments, the second epitope is on a second antigen different from CLDN18.2. In certain embodiments, the second antigen comprises a tumor antigen.

In one aspect, the present disclosure provides a nucleic acid sequence encoding the chimeric antigen receptor (CAR) provided herein. In one aspect, the present disclosure provides a cell comprising the nucleic acid sequence provided herein. In one aspect, the present disclosure provides a cell genetically modified to express the CAR.

In one aspect, the present disclosure provides a vector comprising the nucleic acid sequence provided herein.

In one aspect, the present disclosure provides methods for stimulating a T cell-mediated immune response to a CLDN18.2-expressing cell or tissue in a mammal, the method comprising administering to the mammal an effective amount of a cell genetically modified to express the CAR provided herein.

In one aspect, the present disclosure provides methods of treating a mammal having a CLDN18.2 related disease or condition, comprising administering to the mammal an effective amount of a cell provided herein, thereby treating the mammal.

In certain embodiments, the cell is an autologous T cell. In certain embodiments, the CLDN18.2 related disease or condition is cancer. In certain embodiments, the mammal is a human subject. In certain embodiments, the mammal is identified as having a CLDN18.2-expressing cancer cell, optionally the mammal is identified as having a CLDN18.2 high-expressing cancer cell, a CLDN18.2 medium-expressing cancer cell, or a CLDN18.2 low-expressing cancer cell.

### BRIEF DESCRIPTION OF FIGURES

FIG. 1A is a scatter plot showing the binding affinities of 7C12, 11F12, 12E9 and 26G6 to HEK293-CLDN18.2 cells that express human CLDN18.2. FIG. 1B is a scatter plot showing the binding affinities of 59A9, 18B10, 7C12, 12C12 and 11F12 to HEK293-CLDN18.2 cells. FIG. 1C is a scatter plot showing the binding affinities of 7C12, 11F12, 12E9 and 26G6 to NUGC4 cells. FIG. 1D is a scatter plot showing the binding affinities of 59A9, 18B10, 7C12, 12C12 and 11F12 to NUGC4 cells. Names of the cell lines containing “CLDN18.2”, described herewith and in the figures and examples, refer to human CLDN18.2, unless otherwise specified. Mouse CLDN18.2 is abbreviated as “mCLDN18.2”.

FIG. 2A is a scatter plot showing that chimeric antibodies 7C12-C, 11F12-C, 12E9-C bind to HEK293-CLDN18.2 cell with the EC50 around 0.6 μg/ml, and 26G6-C binds to HEK293-CLDN18.2 cell with the EC50 around 1 μg/ml. FIG. 2B is a scatter plot showing that the CDC potency of antibodies 7C12-C, 11F12-C, 12E9-C and 26G6-C are over 2-fold increase than IMAB362. FIG. 2C is a scatter plot showing that 59A9-C has a slightly higher EC50 (1.3 μg/ml) than 18B10-C (1.0 g/ml). FIG. 2D is a scatter plot showing that antibodies 59A9-C and 18B10-C had over 3-fold increase in CDC potency as compared to IMAB362.

FIG. 3A is a scatter plot showing that 18B10-C binds to MKN45-CLDN18.2-high cells with a significant higher affinity than IMAB362.

FIG. 3B is a scatter plot showing that, using MKN45-CLDN18.2 cell, the two curves suggested that 18B10-C had a better ADCC activity than IMAB362. FIG. 3C is a scatter plot showing that 18B10-C binds to MKN45-CLDN18.2-medium cells with a significant higher affinity than IMAB362. FIG. 3D is a scatter plot showing that, in MKN45-CLDN18.2-medium cell, 18B10-C had over 50-fold increase in ADCC potency as measured by EC50 than IMAB362.

FIG. 4A is a scatter plot showing that 3 out of 4 chimeric antibodies bind to NUGC4 cell with EC50 around 10 μg/ml, except for 26G6-C. FIG. 4B is a scatter plot showing that ADCC activity of 7C12-C, 11F12-C, 12E9-C and 26G6-C chimeric antibodies against NUGC4 cells. FIG. 4C is a scatter plot showing that 18B10-C chimeric antibody binds to NUGC4 cell with EC50 around 10 μg/ml, but not for 59A9-C. FIG. 4D is a scatter plot showing that ADCC activity of 59A9-C and 18B10-C chimeric antibodies against NUGC4 cells in a separate experiment.

FIG. 5 is a bar graph showing the selective binding of 18B10-C and IMAB362 to CLDN18.2—or CLDN18.1-expressing HEK293 cell.

FIGS. 6A and 6B are scatter plots showing the binding affinity when hybridoma antibody 18B10 competes with 10 μg/ml 1MAB362 on MKN 45-CLDN 18.2-high cells and 5 μg/ml 18B10-C on MKN45-CLDN18.2-high cells, respectively. Hybridoma antibody 18B10 could completely block the binding of IMAB362 to MKN45-CLDN18.2-high cells.

FIG. 7A-B are bar graphs showing binding signal of chimeric antibodies to mutated hCLDN 18.2 variants using epitope mapping. Binding of 18B10-C was completely lost when E56 was mutated to Q. This change also applied to IMAB362 and other chimeric antibodies, except for 59A9-C. Other amino acids, such as A42, N45, also contributed to binding of IMAB362 and other antibodies at some extent but not so for 18B10-C

FIG. 8A is a scatter plot showing the binding ability of all the humanized variants along with their chimeric counterparts. FIG. 8B is a scatter plot showing the binding ability of humanized antibody 18B10-HaLa compared to IMAB362 and control hlgG1. 18B10-HaLa binds well to mouse CLDN18.2 with a better potency and a higher MFI than IMAB362.

FIG. 9 is a scatter plot showing the CDC effect of humanized antibody 18B10-HaLa on HEK293-CLDN18.2. 18B10-HaLa has more than 20-fold higher CDC activity than IMAB362.

FIG. 10A is a scatter plot showing the binding affinity of humanized variants of 18B10 relative to chimeric 18B10 for MKN45 cells expressing intermediate levels of CLDN18.2 protein (MKN45-CLDN18.2-medium). All humanized variants of 18B10 bind to MKN45-CLDN18.2-medium cells with a comparable affinity with chimeric 18B10. FIG. 10B is a scatter plot showing the ADCC reporter assay of 18B10-HaLa and IMAB362 on MKN45-CLDN 18.2-medium cells. 18B10-HaLa has a much lower EC50 (0.05 μg/ml) than IMAB362, consistent with that of chimeric 18B10.

FIG. 11A is a scatter plot showing the result of the binding affinity of 18B10-HaLa to NUGC4 cells. FIG. 11B is a scatter plot showing the ADCC effect of 18B10-HaLa compared to IMAB362. 18B10-HaLa has much better ADCC potency than IMAB362.

FIG. 12 is a scatter plot showing the results of the ADCC assay using PBMC (Donor ID:A18Z017017) as the effector cell. 18B10-HaLa shows much better ADCC potency than IMAB362.

FIG. 13A is a bar graph showing the result of 18B10-HaLa epitope mapping (Ab conc: 10 μg/ml). FIG. 13B is a bar graph showing the result of 59A9-C epitope mapping (Ab conc: 10 μg/ml).

FIG. 14A is a scatter plot showing the ADC cytotoxicity of both 18B10-HaLa-vcMMAE and IMAB362-vcMMAE on HEK293-CLDN18.2 cell. Both 18B10-HaLa-vcMMAE and IMAB362-vcMMAE but not the control hIgG1-vcMMAE induced cytotoxicity on HEK293-CLDN18.2 cell. FIG. 14B is a scatter plot showing the result of the ADC cytotoxic effect of 18B10-HaLa-MMAE and IMAB362-MMAE on NUGC-4. 18B10-HaLa-vcMMAE demonstrated a dose-dependent cell growth inhibition starting at a concentration of 0.03 μg/ml, whereas IMAB362-vcMMAE only inhibited cell growth at 10 μg/ml, a much higher concentration. FIG. 14C is a scatter plot showing the ADC cytotoxicity of 18B10-HaLa-MMAE and IMAB362-MMAE on MKN45-CLDN18.2-high. 18B10-HaLa-vcMMAE reached to a maximum cell killing of 86%, which was also higher than IMAB362 (60%).

FIG. 15 is a scatter plot showing the change in tumor volume in relation to time for the isotype control, IMAB362 and 18B10-HaLa. 18B10-HaLa shows a significantly better anti-tumor activity than IMAB362 or isotype control as measured by the tumor size and the TGI (tumor growth inhibition).

FIG. 16 is a scatter plot showing the change in tumor volume in relation to time of the model group (without PBMC), isotype control, 18B10-HaLa 3 mg/kg (mpk) and 18B10-HaLa 10 mpk, respectively. 18B10-HaLa either at 3 mpk or 10 mpk had significant inhibition on tumor growth relative to isotype control or PBMC control.

FIG. 17 is a scatter plot showing the change in tumor volume in relation to time of the isotype control, 18B10-HaLa 0.1 mpk, 18B10-HaLa 0.3 mpk and 18B10-HaLa 1 mpk, respectively. The results indicate that the anti-tumor activity of 18B10-HaLa is dose-dependent.

FIGS. 18A-I are scatter plots showing 18B10-HaLa hIgG1 binding to hFcγRI-his, hFcγRIIB-his, hFcγRIIIA (F176)-his, hFcγRIIIA (V176)-his, mouse FcγRI-his, mouse FcγRIIB-his, mouse FcγRIIIA-his, FcγRIV-his and Cyno FcγRIII-his, mouse FcγRlllA-his, FcγRlV-his and Cyno FcγRlll-his. There was no significant difference between 18B10-HaLa_VLPYLL and 18B10-HaLa-wt in binding to human FcγRI or FcγRIIB. However 18B10-Hala_VLPYLL showed 10-fold increased binding to human FcγRIIIA (F176) and FcγRIIIA (V176) as compared to its wild-type (wt) one. The similar results were shown with mouse FcγRs and cyno FcγRs.

FIGS. 19A-19B are scatter plots showing the binding affinity of 18B10 HaLa hlgG1 to huFcRn-biotin and human C1q, respectively. Results in FIG. 19A indicates that there is no significant difference in FcRn binding between 18B10-HaLa_VLPYLL and 18B10-HaLa wt. Results in FIG. 19B indicates that 18B10-HaLa_VLPYLL has the slightly better binding signal at a lower C1q concentration than that of 18B10-HaLa wt.

FIG. 20A is a scatter plot showing the result of the 18B10-HaLa hlgG1 reporter assay on NUGC-4 (E/T ratio=6:1) using Jurkat-NFAT-luc-FcγRIIIA-V176 as the effector cells. 18B10-HaLa-VLPYLL shows a 3-fold increase in ADCC potency (EC50-0.0097 μg/ml) as compared to that of 18B10-HaLawt (EC50-0.032 μg/ml). FIG. 20B is a scatter plot showing the result of the NUGC-4 ADCC assay using PBMC (Donor ID: A18Z017017) as the effector cells. 18B10-HaLa-VLPYLL shows a 3-fold increase of ADCC potency as compared to that of 18B10-HaLawt and 100-fold increase of ADCC potency as compared to that of IMAB362.

FIG. 21 shows comparison of CLDN18.2 expression level in different gastric cancer cell lines.

FIG. 22A-22D are scatter plots showing the ADCC assays of different gastric cancer cell lines with different CLDN18.2 expression levels.

FIG. 23 is a scatter plot showing the result of the ADCC reporter assay on NUGC4 (E/T ratio=6:1). The antibody produced using the process with the addition of 50 μM 2F—O—F increased the ADCC activity by over 30-fold than that of reference sample produced using a process without the addition of 2F—O—F, or over 1000-fold higher ADCC activity than that of IMAB362.

FIG. 24A-24C are scatter plots showing the FACS binding of different gastric cancer cell lines using 18B10-HaLa low fucose.

FIG. 25A-25E are scatter plots showing the results of ADCC reporter assay on different gastric cancer cell lines using 18B10-HaLa low fucose.

FIG. 26A-26D are scatter plots showing the result of ADCC assay on different gastric cancer cell lines using PBMC (Donor ID: A19028011) as effector cell and 50 μM 2F—O—F sample of 18B10-HaLa.

FIG. 27 shows specific cytotoxicity of 18B10-HaLa low fucose on ADCC assay on NUGC-4 cells.

FIG. 28A to 28B shows Tumor Growth Inhibition of the 18B10-HaLa low fucose at different doses in MKN45-CLDN18.2-high and hPBMC co-inoculation xenograft tumor model.

FIG. 29 shows Tumor Growth Inhibition of 18B10-HaLa low fucose combination with Oxaliplatin and 5-FU on MKN45-CLDN18.2-high tumor model.

FIGS. 30A and 30B shows Tumor Growth Inhibition of antibodies in MKN45-CLDN18.2-high xenograft tumor model.

FIGS. 31A, 31B and 31C show Efficacy of 18B10-HaLa low fucose combined with Paclitaxel in GC02-0004 PDX tumor model in nude mice.

FIGS. 31D and 31E show Tumor Growth Inhibition of antibodies in GC02-0004 PDX tumor model.

FIG. 32 shows Tumor Growth Inhibition (TGI) of antibodies in MKN45-CLDN18.2 xenograft model.

FIGS. 33A and 33B show FACS binding to pancreatic cancer cell lines using 18B10-HaLa low fucose.

FIGS. 34A and 34B show ADCC reporter assay on pancreatic cancer cell lines using Jurkat-NFAT-luc-Fc RIIIA-V176 as the effector cells.

FIG. 35 shows Tumor Growth Inhibition (TGI) of antibodies in MIA PaCa-2-CLDN18.2 xenograft model.

FIG. 36 shows Tumor Growth Inhibition (TGI) of antibodies in BxPC-3-CLDN18.2 xenograft model.

FIGS. 37A and 37B show FACS binding to lung cancer cell lines using 18B10-HaLa low fucose.

FIG. 38 show ADCC reporter assay on NCI-H146 using Jurkat-NFAT-luc-Fc RIIIA-V176 as the effector cells.

FIG. 39 show ADCC assay on NCI-H460-CLDN18.2 using PBMC as the effector cells.

FIGS. 40A and 40B show Tumor Growth Inhibition (TGI) of antibodies in NCI-H146 and PBMC co-inoculation Model.

FIG. 41 shows Tumor Growth Inhibition (TGI) of antibodies in NCI-H460-CLDN18.2 tumor model.

FIG. 42 shows FACS binding to colon cancer cell lines using 18B10-HaLa low fucose.

FIG. 43 shows ADCC reporter assay on colon cancer cell lines using Jurkat-NFAT-luc-FcγRIIIA-V176 as the effector cells.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as λ or κ, while each light chain consists of a variable region (V_{L}) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding domains disclosed herein may be defined or identified by the conventions of Kabat, IMGT, AbM, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J.Mol.Biol., 196,901 (1987); N. R. Whitelegg et al, Protein Engineering, v13(12), 819-824 (2000); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain). In certain embodiments, the antibody provided herein encompasses any antigen-binding fragments thereof.

As used herein, the term “antigen-binding fragment” refers to an antibody fragment formed from a fragment of an antibody comprising one or more CDRs, or any other antibody portion that binds to an antigen but does not comprise an intact native antibody structure. Examples of antigen-binding fragment include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular human antibody.

“Fab” with regard to an antibody refers to a monovalent antigen-binding fragment of the antibody consisting of a single light chain (both variable and constant regions) bound to the variable region and first constant region

of a single heavy chain by a disulfide bond. Fab can be obtained by papain digestion of an antibody at the residues proximal to the N-terminus of the disulfide bond between the heavy chains of the hinge region.

“Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)₂” refers to a dimer of Fab′ that comprises two light chains and part of two heavy chains.

“Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, ADCP and CDC, but does not function in antigen binding.

“Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879 (1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'S of one moiety coordinate with the V_{L}'S of the other moiety and form two binding sites which can target the same antigens (or eptipoes) or different antigens (or eptipoes). In other embodiments, a “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231 (1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub Jun. 15, 2007 (2007)). “Diabodies” include small antibody fragments with two antigen-binding sites, wherein the fragments comprise a V_{H} domain connected to a V_{L} domain in a single polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more V_{H} domains are covalently joined with a peptide linker to form a bivalent or multivalent domain antibody. The two V_{H} domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or “dsFv-dsFv′” comprises three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

The term “humanized” as used herein means that the antibody or antigen-binding fragment comprises CDRs derived from non-human animals, FR regions derived from human, and when applicable, constant regions derived from human. In certain embodiments, the amino acid residues of the variable region framework of the humanized CLDN18.2 antibody are substituted for sequence optimization. In certain embodiments, the variable region framework sequences of the humanized CLDN18.2 antibody chain are at least 65%, 70%, 75%, 80%, 85%, 90%, 95% or 100% identical to the corresponding human variable region framework sequences.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

The term “germline sequence” refers to the nucleic acid sequence encoding a variable region amino acid sequence or subsequence that shares the highest determined amino acid sequence identity with a reference variable region amino acid sequence or subsequence in comparison to all other known variable region amino acid sequences encoded by germline immunoglobulin variable region sequences. The germline sequence can also refer to the variable region amino acid sequence or subsequence with the highest amino acid sequence identity with a reference variable region amino acid sequence or subsequence in comparison to all other evaluated variable region amino acid sequences. The germline sequence can be framework regions only, complementarity determining regions only, framework and complementarity determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. Germline sequences can be determined, for example, through the publicly available international ImMunoGeneTics database (IMGT) and V-base.

“Anti-CLDN18.2 antibody” or “an antibody against CLDN18.2” as used herein refers to an antibody that is capable of specific binding to CLDN18.2 (e.g. human or non-human CLDN18.2) with a sufficient affinity, for example, to provide for diagnostic and/or therapeutic use.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. In certain embodiments, the antibodies or antigen-binding fragments provided herein specifically bind to human and/or non-human CLDN18.2 with a binding affinity (K_{D}) of 10⁻⁶ M (e.g., ≤5×10⁻⁷ M, ≤2×10⁻⁷ M, 10⁻⁷ M, ≤5×10⁻⁸ M, ≤2×10⁻⁸ M, ≤10⁻⁸ M, ≤5×10⁻⁹ M, ≤4×10⁻⁹ M, ≤3×10⁻⁹ M, ≤2×10⁻⁹ M, or ≤10⁻⁹ M. K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry method. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum correspondence. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm. In certain embodiments, the non-identical residue positions may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference.

As used herein, a “homologue sequence” and “homologous sequence” are used interchangeable and refer to polynucleotide sequences (or its complementary strand) or amino acid sequences that have sequences identity of at least 80% (e.g. at least 85%, 88%, 90%, 91%, 92%, 93%, 94%, 95%, 96%, 97%, 98%, 99%) to another sequences when optionally aligned.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An isolated “nucleic acid” or “polynucleotide” are used interchangeably and refer to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The ability to “block binding” or “compete for the same epitope” as used herein refers to the ability of an antibody or antigen-binding fragment to inhibit the binding interaction between two molecules (e.g. human CLDN18.2 and an anti-CLDN18.2 antibody) to any detectable degree. In certain embodiments, an antibody or antigen-binding fragment that blocks binding between two molecules inhibits the binding interaction between the two molecules by at least 50%. In certain embodiments, this inhibition may be greater than 60%, greater than 70%, greater than 80%, or greater than 90%.

The term “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the antibody drug conjugate. Additionally, the antibody drug conjugate can be provided in the form of a fusion protein that may be expressed from a polynucleotide encoding the conjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Except when noted, the terms “patient” or “subject” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Effector functions” or “antibody effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody dependent cell mediated phagocytosis (ADCP), where nonspecific cytotoxic cells that express FcγRs recognize bound antibody on a target cell and subsequently cause phagocytosis of the target cell. Effector functions include both those that operate after the binding of an antigen and those that operate independent of antigen binding.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or antigen-binding fragment thereof, at least one promoter (e.g. SV40, CMV, EF-1α) operably linked to the nucleic acid sequence, and at least one selection marker.

The “host cell” as used herein refers to a cell into which an exogenous polynucleotide and/or a vector has been introduced.

The term “CLDN18.2” refers to Claudin-18 splice variant 2 derived from mammals, such as primates (e.g. humans, monkeys) and rodents (e.g. mice). In certain embodiments, CLDN18.2 is human CLDN18.2. Exemplary sequence of human CLDN18.2 includes human CLDN18.2 protein (NCBI Ref Seq No. NP_001002026.1, or SEQ ID NO: 30). Exemplary sequence of CLDN18.2 includes Mus musculus (mouse) CLDN18.2 protein (NCBI Ref Seq No. NP_001181852.1), Macaca fascicularis (crab-eating macaque) CLDN18.2 protein (NCBI Ref Seq No. XP_015300615.1). CLDN18.2 is expressed in a cancer cell. In one embodiment said CLDN18.2 is expressed on the surface of a cancer cell.

The term “CLDN18.1” refers to Claudin-18 splice variant 1 derived from mammals, such as primates (e.g. humans, monkeys) and rodents (e.g. mice). In certain embodiments, CLDN18.1 is human CLDN18.1. Exemplary sequence of human CLDN18.1 includes human CLDN18.1 protein (NCBI Ref Seq No. NP_057453.1, or SEQ ID NO: 31), Mus musculus (mouse) CLDN18.2 protein (NCBI Ref Seq No. NP_001181851.1), Macaca fascicularis (crab-eating macaque) CLDN18.2 protein (NCBI Ref Seq No. XP_005545920.1).

A “CLDN18.2-related” disease or condition as used herein refers to any disease or condition caused by, exacerbated by, or otherwise linked to increased or decreased expression or activities of CLDN18.2. In some embodiments, the CLDN18.2 related condition is, for example, cancer.

“Cancer” as used herein refers to any medical condition characterized by malignant cell growth or neoplasm, abnormal proliferation, infiltration or metastasis, and includes both solid tumors and non-solid cancers (e.g. hematologic malignancies) such as leukemia. As used herein “solid tumor” refers to a solid mass of neoplastic and/or malignant cells. The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

Anti-CLDN18.2 Antibodies

The present disclosure provides anti-CLDN18.2 antibodies and antigen-binding fragments thereof. The anti-CLDN18.2 antibodies and antigen-binding fragments provided herein are capable of specifically binding to CLDN18.2 (e.g. human CLDN18.2) or CLDN18.2-expressing cells. “Specifically binding” as used herein means a binding affinity (e.g. K_{D}) of ≤10⁻⁶ M (e.g., ≤5×10⁻⁷ M, ≤2×10⁻⁷ M, ≤10⁻⁷ M, ≤5×10⁻⁸ M, 2×10⁻⁸ M, ≤10⁻⁸ M, ≤5×10⁻⁹ M, ≤4×10⁻⁹ M, ≤3×10⁻⁹ M, ≤2×10⁻⁹ M, or ≤10⁻⁹ M).

i. Binding Affinity

Binding affinity of the anti-CLDN18.2 antibodies and antigen-binding fragments provided herein can be represented by K_{D} value, which represents the ratio of dissociation rate to association rate (k_{off}/kₒₙ) when the binding between the antigen and antigen-binding molecule reaches equilibrium. Low-affinity antibodies generally bind antigen slowly and tend to dissociate readily, whereas high-affinity antibodies generally bind antigen faster and tend to remain bound longer. The antigen-binding affinity (e.g. K_{D}) can be appropriately determined using any suitable methods known in the art, including, for example, Kinetic Exclusion Assay (KinExA), or flow cytometry.

In certain embodiments, the “Kd” or “Kd value” according to the present disclosure is in an embodiment measured by KinExA assay, performed with the anti-CLDN18.2 antibody and CLDN18.2 as described by the following assay that measures solution binding affinity of an anti-CLDN18.2 antibody. In general, the KinExA works by equilibrating a constant amount of one binding partner (CBP) with a varying concentration of the other binding partner (Titrant), and then capture a portion of the free CBP by fluorescence labeled secondary antibody in a short contact time which is less than the time needed for dissociation of the pre-formed CBP-Titrant complex. The fluorescence signals generated from the captured CBP are directly proportional to the concentration of free CBP in the equilibrated samples, and are used to generate a binding curve (percent free CBP vs. total Titrant concentration) when measured in a series. More details are available from Schreiber, G., Fersht, A. R. Nature Structural Biology. 1996, 3(5), 427-431. When anti-CLDN18.2 antibody is used as CBP with a constant amount, then CLDN18.2 expressing cell can be used as the Titrant, or vice versa. CLDN18.2 or CLDN18.2-expressing cells can be used in measuring Kd by KinExA. In certain embodiments, the Kd of the anti-CLDN18.2 antibody or antigen-binding fragments thereof is determined in accordance to the method as described in section 3 of Example 10 in the present disclosure.

Other methods suitable for measurement of Kd may also be used under applicable circumstances, for example, radiolabelled antigen-binding assay (see, e.g. Chen, et al., (1999) J. Mol Biol 293:865-881), or surface plasmon resonance assays such as BIAcore using immobilized CLDN18.2 CM5 chips at a proper response units (RU).

In certain embodiments, the binding affinity of the anti-CLDN18.2 antibody is measured by flow cytometry. In general, CLDN18.2-expressing cells are incubated with a range of concentrations of an anti-CLDN18.2 antibody, followed by incubation with a fluorescently labelled secondary antibody, and then analyzed for fluorescent signal intensity. In certain embodiments, the binding affinity of the anti-CLDN18.2 antibody or antigen-binding fragments thereof is determined in accordance to the method as described in Example 5 in the present disclosure.

In certain embodiments, the anti-CLDN18.2 antibodies and the antigen-binding fragments thereof provided herein specifically bind to human CLDN18.2 (or a cell expressing human CLDN18.2) at a K_{D} value of no more than 2.5 nM (or no more than 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4 nM) as measured by KinExA assay.

Alternatively, binding affinity of the anti-CLDN18.2 antibodies and antigen-binding fragments provided herein to human CLDN18.2 can also be represented by “half maximal effective concentration” (EC₅₀) value, which refers to the concentration of an antibody where 50% of its maximal effect (e.g., binding) is observed. The EC₅₀ value can be measured by methods known in the art, for example, sandwich assay such as ELISA, Western Blot, flow cytometry assay, and other binding assay. In certain embodiments, the anti-CLDN18.2 antibodies and the fragments thereof provided herein specifically bind to human CLDN18.2 (e.g. a cell expressing human CLDN18.2) at an EC50 value of no more than 70 μg/ml (or no more than 65, 60, 55, 50, 45, 40, 35, 30, 25, 20, 15, 12, or 10, 9, 8, 7, 6, 5, 4, 3, 2, or 1 g/ml) as measured by flow cytometry.

The binding affinity can be determined with respect to recombinant CLDN18.2, or CLDN18.2-expressing cell lines. The antibody and antigen-binding fragment provided herein are capable of binding to cells expressing different levels of human CLDN18.2, in particular those expressing relatively medium or low levels of human CLDN18.2.

In certain embodiments, the binding affinity is determined with a human CLDN18.2 expressing cell, such as a NUGC4 cell, SNU-620 cell, SNU-601 cell, KATOIII cell, or a comparable cell thereof having a human CLDN18.2 protein expression level comparable to or no more than that of NUGC4 cell, SNU-620 cell, SNU-601 cell, or KATOIII cell.

NUGC4 cell is a cell line established from paragastric lymph node from a cancer patient (see, Akiyama S et al, Jpn J Surg. 1988 July; 18(4):438-46). NUGC4 cell line is available from JCRB Cell Bank under the accession number JCRB0834.

SNU-601 cell and SNU-620 cell both are human stomach carcinoma cell lines established from ascites of cancer patients by Seoul National University (SNU) (KU J L et al, Cancer Res Treat. 2005 February; 37(1): 1-19; Park et al., Int J Cancer. Feb. 7, 1997; 70(4):443-449). SNU-601 cell and SNU-620 cell are available from Korean Cell Line Bank under the accession numbers of 00601 and 00620, respectively.

KATO III cell is a cell line derived from metastatic site of a gastric cancer patient (see, Sekiguchi M, et al. Jpn. J. Exp. Med. 48: 61-68, 1978). KATO III cell line is available from ATCC under the accession number ATCC HTB-103.

Cell lines recombinantly expressing human CLDN18.2 protein can also be established, for example, by transfecting and expressing DNA encoding human CLDN18.2 in a cell line such as Chinese Hamster Ovary (CHO), HEK cells or MKN45 cell line (National Infrastructure of Cell Line Resource, Cat #3111C0001CCC000229), among others.

In certain embodiments, the binding affinity is determined with a human CLDN18.2 high-expressing cell, a human CLDN18.2 medium-expressing cell, or a human CLDN18.2 low-expressing cell.

Expression levels of human CLDN18.2 protein may vary in different cell lines. Expression level of CLDN18.2 protein in a cell can be measured by any suitable methods known in the art, for example, by quantitative fluorescence cytometry or Immunohistochemistry (IHC). In certain embodiments, the expression level of human CLDN18.2 protein on a given cell is determined in accordance to IHC. IHC involves detecting antigens (e.g. CLDN18.2) in cells or tissues by visualizing the antigen by antigen-antibody interaction. Normally, the antigen is detected with a primary antibody against the antigen. The primary antibody may be labelled, to allow direct detection of the antigen. Alternatively, the primary antibody may be unlabeled, and further contacted with a secondary antibody conjugated with a detectable label, to allow indirect detection of the antigen. The primary antibody can be any antibody capable of specifically binding to human CLDN18.2, for example, without limitation, any of the anti-CLDN18.2 antibodies provided herein, or any anti-CLDN18.2 antibodies known in the art. In certain embodiments, the cells or tissues may be fixed, for example, using paraformaldehyde.

The term “high-expressing” as used herein with respect to human CLDN18.2 expressing cells, is intended to mean that the cells expressing human CLDN18.2 at an intensity of at least 2+ as measured by IHC and at a level where at least 40% (e.g. at least 45%, at least 50%, at least 55%, at least 60%, at least 65%, at least 70%, at least 75%, at least 80%, at least 85%, at least 90%, at least 95%, 40-100%, 50-100%, 60-100%, 70-100%, 80-100%, 90-100%, 40-90%, 50-90%, 60-90%, 70-90%, 80-90%, 40-80%, 40-70%, 40-60%, 40-50%, 50-80%, 50-70%, 50-60%, 60-80%, 60-70%, or 70-80%) of the cells are stained positive in IHC. Similarly, the term “medium-expressing” as used herein means that the cells expressing human CLDN18.2 at an intensity of at least 1+ and below 2+ as measured by IHC and at a level where at least 30% (or at least 35%) but below 40% of the cells are stained positive in IHC. Further, the term “low-expressing” as used herein means that the cells expressing human CLDN18.2 at an intensity of above 0 but below 1+ as measured by IHC and at a level where above 0 but below 30% (e.g. 5%, 10%, 15%, 20%, 25%, 5-25%, 10-25%, 15-25%, 20-25%, 5-20%, 5-15%, 5-10%, 10-20%, or 10-15%) of the cells are stained positive in IHC. The definition is also shown in below Table A.

TABLE A
Category of expressing cells

| Category | Intensity | % of positive | Examples of cells |
|---|---|---|---|
| | | cells | |
| High-expressing | at least 2+ | at least 40% | MKN45-CLDN18.2- |
| | | | high; |
| | | | HEK293-CLDN18.2 |
| Medium-expressing | below 2+, | below 40%, at | NUGC4 |
| | at least 1+ | least 30% | |
| Low-expressing | below 1+, | below 30%, | SNU-601; |
| | above 0 | above 0 | SNU-620; |
| | | | KATO III; |
| | | | MKN45-CLDN18.2- |
| | | | medium |

In certain embodiments, human CLDN18.2 expression level is determined by IHC as described in section 6 and section 7 of Example 15. Briefly, cells expressing human CLDN18.2 are fixed in paraffin, and detected via IHC using an anti-human CLDN18.2 antibody, followed by determination of the relative proportion of positively-stained cells and the staining intensity on the cell membrane. In certain embodiments, the cells are stained with a biotinylated anti-CLDN18.2 antibody GC182 in the IHC process. The antibody GC182 has a heavy chain variable region sequence of SEQ ID NO: 74 and a light chain variable region sequence of SEQ ID NO: 75 (see also, WO2013167259).

Based on the immunohistochemical (IHC) determination results provided herein (Table 13), NUGC4 cell can be characterized as a human CLDN18.2 medium-expressing cell line, while SNU-620, SNU-601 and KATOIII cells as low-expressing cell line. In addition, recombinant cell lines may be made to high-express human CLDN18.2. Examples of high-expressing cells include, without limitation, the MKN45-CLDN18.2-high cell line, and the HEK293-CLDN18.2 cell line as described herein in Section 3 of Example 1.

It has been surprisingly found by the inventors that the anti-CLDN18.2 antibodies and the fragments thereof provided herein have high affinity to human CLDN18.2 medium-expressing cell lines (e.g. NUGC4 cell), low-expressing cell lines (e.g. SNU-620, SNU-601 and KATOIII cells). This distinguished from existing antibodies such as IMAB362, which fails to show specific or comparable binding to human CLDN18.2 low-expressing cells. The chimeric IgG1 antibody IMAB362 is an anti-human CLDN18.2 antibody developed by Ganymed Pharmaceuticals AG, having an amino acid sequence disclosed in U.S. patent application US2009169547A1 (IMB362's heavy and light chain variable region sequences are included herein as SEQ ID NO: 72 and SEQ ID NO: 73) and CAS number of 1496553-00-4. IMAB362 recognizes the first extracellular domain (ECD1) of CLDN18.2 and does not bind to any other claudin family member including the closely related splice variant 1 of Claudin 18 (CLDN18.1).

In certain embodiments, the anti-CLDN18.2 antibodies and the fragments thereof provided herein specifically bind to a human CLDN18.2 expressing cell (e.g. NUGC4 cell line or KATOIII cell line) at a K_{D} value of no more than 2.5 nM (or no more than 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4 nM) as measured by KinExA assay. In certain embodiments, the anti-CLDN18.2 antibodies and the fragments thereof provided herein specifically bind to a human CLDN18.2 expressing cell at a Kd value no more than 80%, 70%, 60%, 50%, 40%, 30%, 20%, 15% of that of IMAB362, as measured by KinExA assay. In certain embodiments, the K_{D} value is determined with NUGC4 cell, KATOIII cell, SNU-601 cell, SNU-620 cell or a comparable cell thereof having a human CLDN18.2 protein expression level comparable to or no more than that of NUGC4 cell, KATOIII cell, SNU-601 cell, or SNU-620 cell. In certain embodiments, the K_{D} value is determined with a human CLDN18.2 high-expressing cell line or human CLDN18.2 medium-expressing cell line.

In certain embodiments, the antibody and antigen-binding fragment provided herein has an EC50 value for binding to a human CLDN18.2 (or a mouse CLDN18.2) expressing cell is no more than 70 μg/ml (or no more than 65, 60, 55, 50, 45, 40, 35, 30, 25, 20, 15, 12, or 10, 9, 8, 7, 6, 5, 4, 3, 2, or 1 μg/ml), as measured by flow cytometry assay. In certain embodiments, the antibody and antigen-binding fragment provided herein specifically bind to a human CLDN18.2 expressing cell at an EC50 value no more than 80%, 70%, 60%, 50%, 40%, 30%, 20%, 15%, 10%, 1%, or 0.1% of that of IMAB362, as measured by flow cytometry assay. In certain embodiments, the EC50 is determined with NUGC4 cell line, KATOIII cell line, SNU-601 cell line, SNU-620 cell line, or a comparable cell thereof having a human CLDN18.2 protein expression level comparable to or no more than that of NUGC4 cell line, KATOIII cell line, SNU-601 cell line, or SNU-620 cell line, for example, a human CLDN18.2 low-expressing cell line, or a human CLDN18.2 medium-expressing cell line. In certain embodiments, the EC50 is determined with a human CLDN18.2 high-expressing cell line.

In certain embodiments, the antibody and antigen-binding fragment provided herein has an EC50 value of no more than 5, 4, 3 or 2 μg/ml for binding to a human CLDN18.2 high-expressing cell line or human CLDN18.2 medium-expressing cell line.

In certain embodiments, the anti-CLDN18.2 antibody and antigen-binding fragment provided herein has an EC50 value for binding to NUGC4 cells of no more than 70 μg/ml (or no more than 65, 60, 55, 50, 45, 40, 35, 30, 25, 20, 15, 12, or 10, 9, 8, 7, 6, 5, 4, 3, 2, or 1 μg/ml), as measured by flow cytometry assay.

In certain embodiments, the anti-CLDN18.2 antibodies and the antigen-binding fragments thereof do not bind to CLDN18.1 (e.g. human CLDN18.1 or mouse CLDN18.1).

In certain embodiments, the antibodies and antigen-binding fragments thereof are capable of specifically binding to mouse CLDN18.2 (e.g. a cell expressing mouse CLDN18.2) at an EC50 value no more than 1.5 μg/ml as measured by Flow Cytometry. In certain embodiments, the antibodies and antigen-binding fragments thereof bind to mouse CLDN18.2 at an EC50 of 0.1 μg/ml-1.5 μg/ml (e.g. 0.1 μg/ml-1.2 μg/ml, 0.2 μg/ml-1 μg/ml, 0.5 μg/ml-1 μg/ml, 0.6 μg/ml-1 μg/ml, 0.6 μg/ml-0.8 μg/ml, or 0.67 μg/ml) as measured by Flow Cytometry.

1. ADCC and CDC Activity

In certain embodiments, the anti-CLDN18.2 antibody and antigen-binding fragment provided herein are capable of inducing antibody-dependent cell-mediated cytotoxicity (ADCC) activity and/or CDC activity in cells expressing different levels of human CLDN18.2.

As used herein “antibody-dependent cell-mediated cytotoxicity” or “ADCC” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. Lysis of the target cell is extracellular, requires direct cell-to-cell contact, and does not involve complement. ADCC can be viewed as a mechanism to directly induce a variable degree of immediate tumor destruction that leads to antigen presentation and the induction of tumor-directed T-cell responses. In vivo induction of ADCC is believed to lead to tumor-directed T-cell responses and host-derived antibody responses.

Methods for performing ADCC are known in the art. In general, target cells such as CLDN18.2-expressing cells are incubated with a range of concentrations of an anti-CLDN18.2 antibody, and after washing, effector cells such as Fc receptor expressing cells are added to allow ADCC to occur. Cytotoxicity or cell viability is determined at one time point several hours after the mixing of the target cells with effector cells, to quantify the level of ADCC. Cytotoxicity can be detected by the release of a label (e.g., radioactive substrates, fluorescent dyes or natural intracellular proteins such as lactate dehydrogenase (LDH)) from the lysed target cells. In another embodiment, cell viability is determined by the indicator (such as ATP) of metabolically active cells (see, for example, Crouch, S. P. et al. (1993) J. Immunol. Methods 160, 81-8), using a luciferase reporter gene which generates luminescent signal proportional to the number of living cells in culture (i.e. ADCC reporter assay). Examples of effector cells are NK cells, PBMCs, or FcγRIII-expressing cells. In certain embodiments, the ADCC activity of the anti-CLDN18.2 antibody or antigen-binding fragment thereof provided herein is determined in accordance to the methods described in section 2 of Example 7.

“Complement dependent cytotoxicity” or “CDC” is another cell-killing method that can be directed by antibodies by lysing of a target in the presence of complement. IgM is the most effective isotype for complement activation. IgG1 and IgG3 are also both very effective at directing CDC via the classical complement-activation pathway. In this cascade, the formation of antigen-antibody complexes results in the uncloaking of multiple C1q binding sites in close proximity on the CH2 domains of participating antibody molecules such as IgG molecules (C1q is one of three subcomponents of complement C1) complexed with a cognate antigen. These uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. The complement cascade ends in the formation of a membrane attack complex (MAC), which creates pores in the cell membrane that facilitate free passage of water and solutes into and out of the cell.

CDC activity can be determined by a method similar to that for ADCC activity, as discussed above, except that no effector cells are used presence of complement derived from human serum is required. Briefly, the antibody samples were serially diluted in assay medium, and incubated with target cells expressing CLDN18.2 in the presence of human serum complement. After the incubation, cytotoxicity or cell viability is determined by the release of a label from the lysed target cells, or by an indicator (such as ATP) of metabolically active cells. CellTiter-Glo reagent which assays for ATP in metabolically active cells can be used, and the extent of cell lysis can be quantified by measuring intensity of luminescence with a proper reader. In certain embodiments, the CDC activity of the anti-CLDN18.2 antibody or antigen-binding fragment thereof provided herein is determined in accordance to the methods described in section 1 of Example 7.

In certain embodiments, the ADCC or CDC induced cell death via anti-CLDN18.2 antibodies and the antigen-binding fragments thereof provided herein can be determined by loss of membrane integrity as evaluated by uptake of propidium iodide (PI), trypan blue (see Moore et al. Cytotechnology 17:1-11 (1995)) or 7AAD can be assessed relative to untreated cells.

It has been surprisingly found by the inventors that the anti-CLDN18.2 antibodies and the fragments thereof provided herein are capable of inducing ADCC, and/or CDC to a human CLDN18.2 medium-expressing cell line (e.g. NUGC4 cell), or a human CLDN18.2 low-expressing cell line (e.g. SNU-620, SNU-601 cells, and KATOIII cell). This distinguished from the existing antibodies such as IMAB362, which fails to induce ADCC or CDC to such human CLDN18.2 medium-expressing or low-expressing cell line.

In certain embodiments, the anti-CLDN18.2 antibody and antigen-binding fragment provided herein are capable of inducing complement dependent cytotoxicity (CDC) on a cell expressing human CLDN18.2 at an EC50 value of no more than 1 μg/ml (or no more than 0.9, 0.8, 0.7, 0.6, 0.5 μg/ml) as measured by cytotoxicity assay. In certain embodiments, the anti-CLDN18.2 antibodies and the fragments thereof provided herein are capable of inducing CDC on a cell expressing human CLDN18.2 at an EC50 value no more than 80%, 70%, 60%, 50%, 40%, 30%, 20%, 10%, or 5% of that of IMAB362, as measured by cytotoxicity assay. In certain embodiments, CDC is determined with human CLDN18.2 medium-expressing cell line or a human CLDN18.2 high-expressing cell line.

In certain embodiments, the anti-CLDN18.2 antibody and antigen-binding fragment provided herein are capable of inducing antibody-dependent cell cytotoxicity (ADCC) on a cell expressing human CLDN18.2 at an EC50 value of no more than 2 μg/ml (or no more than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 μg/ml) as measured by an ADCC reporter assay. In certain embodiments, the anti-CLDN18.2 antibodies and antigen-binding fragment thereof provided herein induce ADCC on a cell expressing human CLDN18.2 at an EC50 value no more than 80%, 70%, 60%, 50%, 40%, 30%, 20%, 10%, 5%, or 1% of that of IMAB362, or at a total ADCC capacity (e.g. as indicated by the maximum level of ADCC activity observed in a plot of antibody concentration versus ADCC activity level) at least 120%, 150%, 180%, or 200% of that of IMAB362, as measured by an ADCC reporter assay. In certain embodiments, the ADCC is determined with NUGC4 cell line, KATOIII cell line, SNU-601 cell line, SNU-620 cell line or a comparable cell thereof having a human CLDN18.2 protein expression level comparable to or no more than that of NUGC4 cell line, KATOIII cell line, SNU-601 cell line, or SNU-620 cell line, for example, a human CLDN18.2 medium-expressing cell line or a human CLDN18.2 low-expressing cell line.

In certain embodiments, the anti-CLDN18.2 antibody and antigen-binding fragment provided herein are capable of inducing ADCC on NUGC4 cells at an EC50 value of no more than 2 μg/ml (or no more than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 μg/ml) as measured by an ADCC reporter assay.

Epitope

In certain embodiments, the anti-CLDN18.2 antibody or an antigen-binding fragment thereof provided herein binds to an epitope comprising at least one or more (e.g. one, two, three or more) of amino acid residues at positions D28, W30, V43, N45, Y46, L49, W50, R51, R55, E56, F60, E62, Y66, L72, L76, V79 and R80 of human CLDN18.2 having the amino acid sequence of SEQ ID NO: 30.

The term “epitope” as used herein refers to the specific group of atoms or amino acids on an antigen to which an antibody binds. An epitope can include specific amino acids, sugar side chains, phosphoryl or sulfonyl groups that directly contact an antibody. Those skilled in the art will recognize that it is possible to determine, without undue experimentation, if an antibody binds to the same or overlapping or adjacent epitope as the antibody of present disclosure (e.g., hybridoma/chimeric or humanized antibodies 7C12, 11F12, 26G6, 59A9, 18B10 and any of the chimeric and humanized variant thereof provided herein) by ascertaining whether the two competes for binding to a CLDN18.2 antigen polypeptide.

The term “compete for binding” as used herein with respect to two antigen-binding proteins (e.g. antibodies), means that one antigen-binding protein blocks or reduces binding of the other to the antigen (e.g., human/mouse CLDN18.2), as determined by a competitive binding assay. Competitive binding assays are well known in the art, include, for example, direct or indirect radioimmunoassay (RIA), direct or indirect enzyme immunoassay (EIA), and sandwich competition assay (see, e.g., Stahli et al., 1983, Methods in Enzymology 9:242-253). Typically, such an assay involves the use of purified antigen bound to a solid surface or cells bearing the antigen, an unlabelled test antibody and a labeled reference antibody. Competitive inhibition is measured by determining the amount of label bound to the solid surface or cells in the presence of the test antibody. Usually the test antibody is present in excess. If two antibodies competes for binding to the CLDN18.2, then the two antibodies bind to the same or overlapping epitope, or an adjacent epitope sufficiently proximal to the epitope bound by the other antibody for steric hindrance to occur. Usually, when a competing antibody is present in excess, it will inhibit (e.g., reduce) specific binding of a test antibody to a common antigen by at least 50-55%, 55-60%, 60-65%, 65-70%, 70-75% 75-80%, 80-85%, 85-90% or more.

In certain embodiments, the epitope or the amino acid residue in the epitope bound by an antibody can be determined by mutating specific residues in the antigen, i.e., CLDN18.2. If an antibody binds to a mutant CLDN18.2 having an amino acid residue mutated, for example to alanine, at significantly reduced level relative to its binding to wild-type CLDN18.2, then this would indicate that the mutated residue is directly involved in the binding of the antibody to CLDN18.2 antigen, or is in close proximity to the antibody when it is bound to the antigen. Such a mutated residue is considered to be within the epitope, and the antibody is considered to specifically bind to an epitope comprising the residue. A significantly reduced level in binding as used herein, means that the binding affinity (e.g. EC50, Kd, or binding capacity) between the antibody and the mutant CLDN18.2 is reduced by greater than 10%, 20%, 30%, 40%, 50%, 60%, 70%, 80%, 90%, or more, relative to the binding between the antibody and a wild type CLDN18.2. Such a binding measurement can be conducted using any suitable methods known in the art and disclosed herein, for example, without limitation, KinExA assay, and flow cytometry.

In certain embodiments, the anti-CLDN18.2 antibody or an antigen-binding fragment thereof provided herein exhibit significantly lower binding for a mutant CLDN18.2 in which a residue in a wild-type CLDN18.2 is substituted with alanine, and the residue is selected from the group consisting of: D28, W30, V43, N45, Y46, L49, W50, R51, R55, E56, F60, E62, Y66, L72, L76, V79 and R80 of human CLDN18.2. In certain embodiments, the residue is E56. In certain embodiments, the residue is selected from the group consisting of: W30, L49, W50, R55, and E56. In certain embodiments, the residue is selected from the group consisting of: T41, N45, Y46, R51, F60, E62, and R80. In certain embodiments, the residue is selected from the group consisting of: D28, V43, N45, Y46, Y66, L72, L76, and V79.

In certain embodiments, the anti-CLDN18.2 antibody or an antigen-binding fragment thereof provided herein exhibit at least 80%, 90%, 95% or 99% or more reduction in binding for a mutant CLDN18.2 comprising E56A of human CLDN18.2, relative to the binding between the antibody and a wild type CLDN18.2.

In certain embodiments, the anti-CLDN18.2 antibody or an antigen-binding fragment thereof provided herein exhibit at least 50%, 60%, 70%, 80%, or 90% reduction in binding for a mutant CLDN18.2 comprising one or more mutated residue selected from the group consisting of: W30A, L49A, W50A, R55A, and E56A of human CLDN18.2, relative to the binding between the antibody and a wild type CLDN18.2.

In certain embodiments, the anti-CLDN18.2 antibody or an antigen-binding fragment thereof provided herein exhibit at least 30%, 35%, 40%, 45%, or 50% reduction in binding for a mutant CLDN18.2 comprising one or more mutated residue selected from the group consisting of: D28, V43, N45, Y46, Y66, L72, L76, and V79 of human CLDN18.2, relative to the binding between the antibody and a wild type CLDN18.2.

In certain embodiments, the anti-CLDN18.2 antibody or an antigen-binding fragment thereof provided herein exhibit at least 10%, 15%, 20%, 25%, or 30% reduction in binding for a mutant CLDN18.2 comprising one or more mutated residue selected from the group consisting of: T41A, N45A, Y46A, R51A, F60A, E62A, and R80A of human CLDN18.2, relative to the binding between the antibody and a wild type CLDN18.2.

In certain embodiments, the anti-CLDN18.2 antibody or an antigen-binding fragment thereof provided herein do not bind to A42, and/or N45.

In certain embodiments, the anti-CLDN18.2 antibody or an antigen-binding fragment thereof provided herein are capable of binding to the epitope provided herein, and inducing ADCC or CDC activity in a human CLDN18.2 medium-expressing cell line or a human CLDN18.2 low-expressing cell line.

Antibody Sequences

In another aspect, the present disclosure provides an anti-CLDN18.2 antibody or an antigen-binding fragment thereof, comprising heavy chain HCDR1, HCDR2 and HCDR3 and/or light chain LCDR1, LCDR2 and LCDR3 sequences, wherein
the HCDR1 sequence comprises GYNMN (SEQ ID NO: 1), or TYFIGVG (SEQ ID NO: 13), or a homologue sequence of at least 80% sequence identity thereof;
the HCDR2 sequence comprises X₁IDPYYX₂X₃TX₄YNQKFX₅G (SEQ ID NO: 32), or HIWWNDNKYYNTALKS (SEQ ID NO: 15), or a homologue sequence of at least 80% (or at least 85%, 90%, 95%) sequence identity thereof;
the HCDR3 sequence comprises X₆X₇X₈GNAFDY (SEQ ID NO: 33), or MGSGAWFTY (SEQ ID NO: 17), or a homologue sequence of at least 80% sequence identity thereof;
the LCDR1 sequence comprises KSSQX₉LX₁₀NX₁₁GNX₁₂KNYLT (SEQ ID NO: 34) or a homologue sequence of at least 80% (or at least 85%, 90%, 95%) sequence identity thereof;
the LCDR2 sequence comprises WASTRX₁₃S (SEQ ID NO: 35) or a homologue sequence of at least 80% sequence identity thereof; and the LCDR3 sequence comprises QNDYX₁₄X₁₅PX₁₆T (SEQ ID NO: 36) or a homologue sequence of at least 80% sequence identity thereof;
wherein X₁ is N or Y or H, X₂ is G or V, X₃ is A or G or T, X₄ is R or T or S, X₅ is K or R, X₆ is S or M, X₇ is Y or F, X₈ is Y or H, X₉ is S or N, X₁₀ is L or F, X₁₁ is S or N, X₁₂ is Q or L, X₁₃ is E or K, X₁₄ is S or Y, X₁₅ is F or Y and X₁₆ is F or L.

In one aspect, the present disclosure provides an anti-CLDN18.2 antibody or an antigen-binding fragment thereof provided herein, wherein the heavy chain variable region comprises:
a) a HCDR1 comprises a sequence selected from SEQ ID NO: 1, and SEQ ID NO: 13,
b) a HCDR2 comprises a sequence selected from SEQ ID NO: 3, SEQ ID NO: 7, SEQ ID NO: 9, SEQ ID NO: 15, SEQ ID NO: 19, and SEQ ID NO: 22, and
c) a HCDR3 comprises a sequence selected from SEQ ID NO: 5, SEQ ID NO: 11, SEQ ID NO: 17, and SEQ ID NO: 21, and/or a light chain variable region comprising:
d) a LCDR1 comprises a sequence of SEQ ID NO: 2, SEQ ID NO: 10, SEQ ID NO: 14, and SEQ ID NO: 20,
e) a LCDR2 comprises a sequence of SEQ ID NO: 4, and SEQ ID NO: 16, and f) a LCDR3 comprises a sequence selected from SEQ ID NO: 6, SEQ ID NO: 8, SEQ ID NO: 12, and SEQ ID NO: 18.

In certain embodiments, the antibody or an antigen-binding fragment thereof provided herein, wherein the heavy chain variable region is selected from the group consisting of:
a) a heavy chain variable region comprising a HCDR1 comprising the sequence of SEQ ID NO: 1, a HCDR2 comprising the sequence of SEQ ID NO: 3, and a HCDR3 comprising the sequence of SEQ ID NO: 5;
b) a heavy chain variable region comprising a HCDR1 comprising the sequence of SEQ ID NO: 1, a HCDR2 comprising the sequence of SEQ ID NO: 7, and a HCDR3 comprising the sequence of SEQ ID NO: 5;
c) a heavy chain variable region comprising a HCDR1 comprising the sequence of SEQ ID NO: 1, a HCDR2 comprising the sequence of SEQ ID NO: 9, and a HCDR3 comprising the sequence of SEQ ID NO: 11;
d) a heavy chain variable region comprising a HCDR1 comprising the sequence of SEQ ID NO: 13, a HCDR2 comprising the sequence of SEQ ID NO: 15, and a HCDR3 comprising the sequence of SEQ ID NO: 17;
e) a heavy chain variable region comprising a HCDR1 comprising the sequence of SEQ ID NO: 1, a HCDR2 comprising the sequence of SEQ ID NO: 19, and a HCDR3 comprising the sequence of SEQ ID NO: 21; and
f) a heavy chain variable region comprising a HCDR1 comprising the sequence of SEQ ID NO: 1, a HCDR2 comprising the sequence of SEQ ID NO: 22, and a HCDR3 comprising the sequence of SEQ ID NO: 5.

In certain embodiments, the antibody or an antigen-binding fragment thereof provided herein, wherein the light chain variable region is selected from the group consisting of:
a) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 6;
b) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 8;
c) a light chain variable region comprising a LCDR1 comprising the sequence of SEQ ID NO: 10, a LCDR2 comprising the sequence of SEQ ID NO: 4, and a LCDR3 comprising the sequence of SEQ ID NO: 6;
d) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 12;
e) a light chain variable region comprising a LCDR1 comprising the sequence of SEQ ID NO: 14, a LCDR2 comprising the sequence of SEQ ID NO: 16, and a LCDR3 comprising the sequence of SEQ ID NO: 18; and
f) a light chain variable region comprising a LCDR1 comprising the sequence of SEQ ID NO: 20, a LCDR2 comprising the sequence of SEQ ID NO: 4, and a LCDR3 comprising the sequence of SEQ ID NO: 6.

In certain embodiments, the antibody or an antigen-binding fragment thereof provided herein, wherein:
a) the heavy chain variable region comprises a HCDR1 comprising the sequence of SEQ ID NO: 1, a HCDR2 comprising the sequence of SEQ ID NO: 3, and a HCDR3 comprising the sequence of SEQ ID NO: 5; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 6;
b) the heavy chain variable region comprises a HCDR1 comprising the sequence of SEQ ID NO: 1, a HCDR2 comprising the sequence of SEQ ID NO: 7, and a HCDR3 comprising the sequence of SEQ ID NO: 5; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 8;
c) the heavy chain variable region comprises a HCDR1 comprising the sequence of SEQ ID NO: 1, a HCDR2 comprising the sequence of SEQ ID NO: 9, and a HCDR3 comprising the sequence of SEQ ID NO: 11; and the light chain variable region comprises a LCDR1 comprising the sequence of SEQ ID NO: 10, a LCDR2 comprising the sequence of SEQ ID NO: 4, and a LCDR3 comprising the sequence of SEQ ID NO: 6;
d) the heavy chain variable region comprises a HCDR1 comprising the sequence of SEQ ID NO: 13, a HCDR2 comprising the sequence of SEQ ID NO: 15, and a HCDR3 comprising the sequence of SEQ ID NO: 17; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 12;
e) the heavy chain variable region comprises a HCDR1 comprising the sequence of SEQ ID NO: 1, a HCDR2 comprising the sequence of SEQ ID NO: 19, and a HCDR3 comprising the sequence of SEQ ID NO: 21; and the light chain variable region comprises a LCDR1 comprising the sequence of SEQ ID NO: 14, a LCDR2 comprising the sequence of SEQ ID NO: 16, and a LCDR3 comprising the sequence of SEQ ID NO: 18; or
f) the heavy chain variable region comprises a HCDR1 comprising the sequence of SEQ ID NO: 1, a HCDR2 comprising the sequence of SEQ ID NO: 22, and a HCDR3 comprising the sequence of SEQ ID NO: 5; and the light chain variable region comprises a LCDR1 comprising the sequence of SEQ ID NO: 20, a LCDR2 comprising the sequence of SEQ ID NO: 4, and a LCDR3 comprising the sequence of SEQ ID NO: 6.

In certain embodiments, the antibodies provided herein comprise one or more (e.g. 1, 2, 3, 4, 5, or 6) CDR sequences of a CLDN18.2 antibodies 7C12, 11F12, 26G6, 59A9, 18B10 and 12E9.

“7C12” as used herein refers to a mouse antibody having a heavy chain variable region of SEQ ID NO: 37, and a light chain variable region of SEQ ID NO: 38.

“11F12” as used herein refers to a mouse antibody having a heavy chain variable region of SEQ ID NO: 39, and a light chain variable region of SEQ ID NO: 40.

“26G6” as used herein refers to a mouse antibody having a heavy chain variable region of SEQ ID NO: 41, and a light chain variable region of SEQ ID NO: 42.

“59A9” as used herein refers to a mouse antibody having a heavy chain variable region of SEQ ID NO: 43, and a light chain variable region of SEQ ID NO: 44.

“18B10” as used herein refers to a mouse antibody having a heavy chain variable region of SEQ ID NO: 45, and a light chain variable region of SEQ ID NO: 46.

“12E9” as used herein refers to a mouse antibody having a heavy chain variable region of SEQ ID NO: 47, and a light chain variable region of SEQ ID NO: 48.

Table 1 showS the CDR sequences of these CLDN18.2 antibodies. The heavy chain and light chain variable region sequences are also provided below in Table 2.

TABLE1
Sequences of CLDN18.2 antibodies' CDR region

| Antibody | Region | CDR1 | CDR2 | CDR3 |
|---|---|---|---|---|
| 7C12 | HCDR | SEQ ID NO: 1 | SEQ ID NO: 3 | SEQ ID NO: 5 |
| | | GYNMN | NIDPYYGATRYNQKFKG | SYYGNAFDY |
| | LCDR | SEQ ID NO: 2 | SEQ ID NO: 4 | SEQ ID NO: 6 |
| | | KSSQSLLNSGNQKNYLT | WASTRES | QNDYSFPFT |
| 11F12 | HCDR | SEQ ID NO: 1 | SEQ ID NO: 7 | SEQ ID NO: 5 |
| | | GYNMN | YIDPYYGGTRYNQKFKG | SYYGNAFDY |
| | LCDR | SEQ ID NO: 2 | SEQ ID NO: 4 | SEQ ID NO: 8 |
| | | KSSQSLLNSGNQKNYLT | WASTRES | QNDYSYPFT |
| 26G6 | HCDR | SEQ ID NO: 1 | SEQ ID NO: 9 | SEQ ID NO: 11 |
| | | GYNMN | HIDPYYVTTTYNQKFRG | SFYGNAFDY |
| | LCDR | SEQ ID NO: 10 | SEQ ID NO: 4 | SEQ ID NO: 6 |
| | | KSSQSLFNSGNQKNYLT | WASTRES | QNDYSFPFT |
| 59A9 | HCDR | SEQ ID NO: 13 | SEQ ID NO: 15 | SEQ ID NO: 17 |
| | | TYFIGVG | HIWWNDNKYYNTALKS | MGSGAWFTY |
| | LCDR | SEQ ID NO: 2 | SEQ ID NO: 4 | SEQ ID NO: 12 |
| | | KSSQSLLNSGNQKNYLT | WASTRES | QNDYYYPLT |
| 18B10 | HCDR | SEQ ID NO: 1 | SEQ ID NO: 19 | SEQ ID NO: 21 |
| | | GYNMN | NIDPYYGGTSYNQKFKG | MYHGNAFDY |
| | LCDR | SEQ ID NO: 14 | SEQ ID NO: 16 | SEQ ID NO: 18 |
| | | KSSQSLLNSGNLKNYLT | WASTRKS | QNDYSYPLT |
| 12E8 | HCDR | SEQ ID NO: 1 | SEQ ID NO: 22 | SEQ ID NO: 5 |
| | | GYNMN | NIDPYYGGTRYNQKFKG | SYYGNAFDY |
| | LCDR | SEQ ID NO: 20 | SEQ ID NO: 4 | SEQ ID NO: 6 |
| | | KSSQNLLNNGNQKNYLT | WASTRES | QNDYSFPFT |

TABLE2
Sequences of mouse/chimeric antibody VH/VL

| | VH | VL |
|---|---|---|
| 7C12 | SEQ ID NO: 37 | SEQ ID NO: 38 |
| | EFQLQQSGPELEKPGASVRISCKTSGYSFT | DIVMTQSPSSLTVTAGEKVTMSCKSSQSLL |
| | GYNMNWVKQSNGESLEWIGNIDPYYGATRY | NSGNQKNYLTWYQQKPGQPPKLLIYWASTR |
| | NQKFKGKATLTVDKSSSTAYMQLKSLTSED | ESGVPDRFTGSGSGTDFTLTISSVQAEDLA |
| | SAVYYCARSYYGNAFDYWGQGTTLTVSS | VYYCQNDYSFPFTFGSGTKLEIK |
| 11F12 | SEQ ID NO: 39 | SEQ ID NO: 40 |
| | EFQLQQSGPELEKPGASVRISCKTSGYSFT | DIVMTQSPSSLTVTAGEKVTMSCKSSQSLL |
| | GYNMNWVKQSNGESLEWIGYIDPYYGGTRY | NSGNQKNYLTWYQQKPGQPPKLLIYWASTR |
| | NQKFKGKATLTVDKSSSTAYMQLKSLTSED | ESGVPDRFTGSGSGTDFTLTISSVQAEDLA |
| | SAVYYCARSYYGNAFDYWGQGTTLTVSS | VYYCQNDYSYPFTFGSGTKLEIK |
| 26G6 | SEQ ID NO: 41 | SEQ ID NO: 42 |
| | EFQLQQSGPELEKPGASVKISCKTSGYSFT | DIVMTQSPSSLTVTAGEKVTMSCKSSQSLF |
| | GYNMNWVKQSNGQSLEWIGHIDPYYVTTTY | NSGNQKNYLTWYQQKPGQPPKLLIYWASTR |
| | NQKFRGKATLTVDKSSSTAYMQLKSLTSED | ESGVPDRFTGSGSGTDFTLTISSVQAEDLA |
| | SAVYYCARSFYGNAFDYWGQGTTLTVSS | VYYCQNDYSFPFTFGSGTKLEIK |
| 59A9 | SEQ ID NO: 43 | SEQ ID NO: 44 |
| | QITQKESGPGILQPSQTLSLTCSLSGFSLS | DIVMTQSPSSLTVTAGEKVTMSCKSSQSLL |
| | TYFIGVGWIRQPSGKGLEWLAHIWWNDNKY | NSGNQKNYLTWYQQKPGQPPKLLIYWASTR |
| | YNTALKSRLTISKDTSNNQVFLKIASVDTA | ESGVPDRFTGSGSGTDFTLTISSVQAEDLA |
| | DTATYYCARMGSGAWFTYWGQGTLVTVSA | VYYCQNDYYYPLTFGSGTKLEIK |
| 18B10 | SEQ ID NO: 45 | SEQ ID NO: 46 |
| | EFQLQQSGPELEKPGASVRISCKTSGYSFT | DIVMTQSPSSLTVTAGEKVTMSCKSSQSLL |
| | GYNMNWVKQSNGESLEWIGNIDPYYGGTSY | NSGNLKNYLTWYQQKPGQPPKLLIYWASTR |
| | NQKFKGKATLTVDKSSSTAYMQLKSLTSED | KSGVPDRFTGSGSGTDFTLTLSSVQAEDLA |
| | SAVYYCARMYHGNAFDYWGQGTTLTVSS | VYYCQNDYSYPLTFGAGTKLELK |
| 12E9 | SEQ ID NO: | SEQ ID NO: 48 |
| | EFQLQQSGPELEKPGASVRISCKTSGYSFT | DIVMTQSPSSLTVTAGEKVTMSCKSSQNLL |
| | GYNMNWVKQSNGESLEWIGNIDPYYGGTRY | NNGNQKNYLTWYQQKPGQPPKLLIYWASTR |
| | NQKFKGKATLTVDKSSSTAYMQLKSLTSED | ESGVPDRFTGSGSGTDFILTISSVQAEDLA |
| | SAVYYCARSYYGNAFDYWGQGTTLTVSS | VYYCQNDYSFPFTFGAGTKLELK |

The anti-CLDN18.2 antibodies or antigen-binding fragments thereof provided herein can be a monoclonal antibody, polyclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals.

CDRs are known to be responsible for antigen binding, however, it has been found that not all of the 6 CDRs are necessarily indispensable or unchangeable. In other words, it is possible to replace or change or modify 1, 2, or 3 CDRs in anti-CLDN18.2 antibodies 7C12, 11F12, 26G6, 59A9, 18B10, or 12E9 (corresponding to any one of SEQ ID NOs: 1-22), yet substantially retain the specific binding affinity to CLDN18.2.

In certain embodiments, the anti-CLDN18.2 antibodies and the antigen-binding fragments provided herein comprise a heavy chain CDR3 sequence of one of the anti-CLDN18.2 antibodies 7C12, 11F12, 26G6, 59A9, 18B10, or 12E9. In certain embodiments, the anti-CLDN18.2 antibodies and the antigen-binding fragments provided herein comprise a heavy chain CDR3 sequence of SEQ ID NOs: 5, 11, 17, and 21. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In some embodiments, the anti-CLDN18.2 antibodies and the antigen-binding fragments provided herein comprise all or a portion of the heavy chain variable domain and/or all or a portion of the light chain variable domain. In one embodiment, the anti-CLDN18.2 antibodies and the antigen-binding fragments provided herein is a single domain antibody which consists of all or a portion of the heavy chain variable domain provided herein. More information of such a single domain antibody is available in the art (see, e.g., U.S. Pat. No. 6,248,516).

In certain embodiments, the antibodies and antigen-binding fragments thereof provided herein comprise suitable framework region (FR) sequences, as long as the antibodies and antigen-binding fragments thereof can specifically bind to CLDN18.2. The CDR sequences provided in Table 1 are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain germline sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody. In some embodiments, the humanized antibody or antigen-binding fragment comprise human heavy/light chain FR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure to reduce or avoid immunogenicity and/or improve or retain the binding activity or binding affinity.

In certain embodiments, the humanized antibody or antigen-binding fragment provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains. In certain embodiments, the one or more amino acid residues are mutated, for example, back-mutated to the corresponding residue found in the non-human parent antibody (e.g. in the mouse framework region) from which the CDR sequences are derived. Suitable positions for mutations can be selected by a skilled person following principles known in the art. For example, a position for mutation can be selected where: 1) the residue in the framework of the human germline sequence is rare (e.g. in less than 20% or less than 10% in human variable region sequence); 2) the position is immediately adjacent to one or more of the 3 CDR's in the primary sequence of the human germline chain, as it is likely to interact with residues in the CDRs; or 3) the position is close to CDRs in a 3-dimensional model, and therefore can have a good probability of interacting with amino acids in the CDR. The residue at the selected position can be mutated back to the corresponding residue in the parent antibody, or to a residue which is neither the corresponding residue in human germline sequence nor in parent antibody, but to a residue typical of human sequences, i.e. that occurs more frequently at that position in the known human sequences belonging to the same subgroup as the human germline sequence (see U.S. Pat. No. 5,693,762).

In certain embodiments, the humanized light and heavy chains of the present disclosure are substantially non-immunogenic in humans and retain substantially the same affinity as or even higher affinity than the parent antibody to CLDN18.2.

In certain embodiments, the humanized antibodies and antigen-binding fragment thereof provided herein comprise one or more light chain FR sequences of human germline framework sequence VK/4-1, and/or one or more heavy chain FR sequences of human germline framework sequence VH/1-46, without or without back mutations. Back mutations can be introduced in to the human germline framework sequence, if needed. In certain embodiments, the humanized antibody 18B10 may contain one or more back mutations selected from the group consisting of: R71I, T73K, T28S, M69L, R38K, and M48I, all based on Kabat numbering, in heavy chain framework sequence VH/1-46. The humanized antibody 18B10 may contain one or more back mutations selected from the group consisting of: S63T, and I21M, all based on Kabat numbering, in light chain framework sequence VK/4-1.

In certain embodiments, the anti-CLDN18.2 antibody or an antigen-binding fragment thereof provided herein, comprises a heavy chain variable region comprising the sequence selected from the group consisting of SEQ ID NO: 25, SEQ ID NO: 27, SEQ ID NO: 29, SEQ ID NO: 37, SEQ ID NO: 39, SEQ ID NO: 41, SEQ ID NO: 43, SEQ ID NO: 45, and SEQ ID NO: 47, and a homologous sequence thereof having at least 80% (e.g. at least 85%, 90%, 95%, 96%, 97%, 98%, or 99%) sequence identity yet retaining specific binding affinity to CLDN18.2, in particular human CLDN18.2.

In certain embodiments, the anti-CLDN18.2 antibody or an antigen-binding fragment thereof provided herein, antibody or an antigen-binding fragment thereof comprises a light chain variable region comprising the sequence selected from the group consisting of SEQ ID NO: 26, SEQ ID NO: 28, SEQ ID NO: 38, SEQ ID NO: 40, SEQ ID NO: 42, SEQ ID NO: 44, SEQ ID NO: 46, SEQ ID NO: 48, and a homologous sequence thereof having at least 80% (e.g. at least 85%, 90%, 95%, 96%, 97%, 98%, or 99%) sequence identity yet retaining specific binding affinity to CLDN18.2, in particular human CLDN18.2.

In certain embodiments, the anti-CLDN18.2 antibody or an antigen-binding fragment thereof provided herein, comprising:
a heavy chain variable region comprising the sequence of SEQ ID NO: 25 and a light chain variable region comprising the sequence of SEQ ID NO: 26;
a heavy chain variable region comprising the sequence of SEQ ID NO: 27 and a light chain variable region comprising the sequence of SEQ ID NO: 28;
a heavy chain variable region comprising the sequence of SEQ ID NO: 29 and a light chain variable region comprising the sequence of SEQ ID NO: 26, or 28;
a heavy chain variable region comprising the sequence of SEQ ID NO: 37 and a light chain variable region comprising the sequence of SEQ ID NO: 38;
a heavy chain variable region comprising the sequence of SEQ ID NO: 39 and a light chain variable region comprising the sequence of SEQ ID NO: 40;
a heavy chain variable region comprising the sequence of SEQ ID NO: 41 and a light chain variable region comprising the sequence of SEQ ID NO: 42;
a heavy chain variable region comprising the sequence of SEQ ID NO: 43 and a light chain variable region comprising the sequence of SEQ ID NO: 44;
a heavy chain variable region comprising the sequence of SEQ ID NO: 45 and a light chain variable region comprising the sequence of SEQ ID NO: 46; or
a heavy chain variable region comprising the sequence of SEQ ID NO: 47 and a light chain variable region comprising the sequence of SEQ ID NO: 48.

In certain embodiments, the anti-CLDN18.2 antibody or an antigen-binding fragment thereof provided herein further comprises one or more of heavy chain HFR1, HFR2, HFR3 and HFR4, and/or one or more of light chain LFR1, LFR2, LFR3 and LFR4, wherein:
the HFR1 comprises QVQLVQSGAEVKKPGASVKVSCKASGYX₁₇FT (SEQ ID NO: 54) or a homologous sequence of at least 80% (or at least 85%, 90%, 95%) sequence identity thereof,
the HFR2 comprises WVX₁₈QAPGQGLEWX₁₉G (SEQ ID NO: 55) or a homologous sequence of at least 80% (or at least 90%) sequence identity thereof, the HFR3 sequence comprises RVTX₂₀TIDKSTSTVYMELSSLRSEDTAVYYCAR (SEQ ID NO: 56) or a homologous sequence of at least 80% (or at least 85%, 90%, 95%) sequence identity thereof,
the HFR4 comprises WGQGTTVTVSS (SEQ ID NO: 57) or a homologous sequence of at least 80% sequence identity thereof, the LFR1 comprises DIVMTQSPDSLAVSLGERATX₂₁NC (SEQ ID NO: 58) or a homologous sequence of at least 80% (or at least 85%, 90%, 95%) sequence identity thereof,
the LFR2 comprises WYQQKPGQPPKLLIY (SEQ ID NO: 59) or a homologous sequence of at least 80% (or at least 85%, 90%) sequence identity thereof, the LFR3 comprises GVPDRFX₂₂GSGSGTDFTLTISSLQAEDVAVYYC (SEQ ID NO: 60) or a homologous sequence of at least 80% (or at least 85%, 90%, 95%) sequence identity thereof, and
the LFR4 comprises FGGGTKVEIK (SEQ ID NO: 61) or a homologous sequence of at least 80% (or at least 90%) sequence identity thereof,
wherein X₁₇ is T or S, X₁₈ is R or K, X₁₉ is M or I, X₂₀ is M or L, X₂₁ is I or M, and X₂₂ is S or T.

In certain embodiments, the HFR1 comprises a sequence selected from the group consisting of SEQ ID NOs: 62 and 63, the HFR2 comprises a sequence selected from the group consisting of SEQ ID NOs: 64 and 65, the HFR3 comprises the sequence selected from the group consisting of SEQ ID NOs: 66 and 67, the HFR4 comprises a sequence of SEQ TD NOs: 57, the LFR1 comprises the sequence from the group consisting of SEQ ID NOs: 68 and 69, the LFR2 comprises a sequence of SEQ ID NO: 59, the LFR3 comprises a sequence selected from the group consisting of SEQ TD NOs: 70 and 71, and the LFR4 comprises a sequence of SEQ ID NO: 61.

TABLE3-1
Framework (FR) sequences of humanized CLDN18.2 antibodies 18B10

| Antibody chain | FR1 | FR2 | FR3 | FR4 |
|---|---|---|---|---|
| Hu18B10-Ha | SEQ ID NO: 62 | SEQ ID NO: 64 | SEQ ID NO: 66 | SEQ ID NO: 57 |
| | QVQLVQSGAEVKKPGA | WVRQAPGQGLEWMG | RVTMTIDKSTSTVYM | WGQGTTVTVSS |
| | SVKVSCKASGYTFT | | ELSSLRSEDTAVYYC | |
| | | | AR | |
| Hu18B10-Hb | SEQ ID NO: 63 | SEQ ID NO: 64 | SEQ ID NO: 67 | SEQ ID NO: 57 |
| | QVQLVQSGAEVKKPGA | WVRQAPGQGLEWMG | RVTLTIDKSTSTVYM | WGQGTTVTVSS |
| | SVKVSCKASGYSFT | | ELSSLRSEDTAVYYC | |
| | | | AR | |
| Hu18B10-Hc | SEQ ID NO: 63 | SEQ ID NO: 65 | SEQ ID NO: 67 | SEQ ID NO: 57 |
| | QVQLVQSGAEVKKPGA | WVKQAPGQGLEWIG | RVTLTIDKSTSTVYM | WGQGTTVTVSS |
| | SVKVSCKASGYSFT | | ELSSLRSEDTAVYYC | |
| | | | AR | |
| Hu18B10_La | SEQ ID NO: 68 | SEQ ID NO: 59 | SEQ ID NO: 70 | SEQ ID NO: 61 |
| | DIVMTQSPDSLAVSLG | WYQQKPGQPPKLLI | GVPDRFSGSGSGTDF | FGGGTKVEIK |
| | ERATINC | Y | TLTISSLQAEDVAVY | |
| | | | YC | |
| Hu18B10_Lb | SEQ ID NO: 69 | SEQ ID NO: 59 | SEQ ID NO: 71 | SEQ ID NO: 61 |
| | DIVMTQSPDSLAVSLG | WYQQKPGQPPKLLI | GVPDFRSGSGSGTDF | FGGGTKVEIK |
| | ERATMNC | Y | TLTISSLQAEDVAVY | |
| | | | YV | |

Table 3-2 illustrates sequences of the variable regions of humanized 18B10 antibodies.

TABLE3-2
Sequences of humanized 18B10

| Antibody | |
|---|---|
| chain | Sequences |
| 18B10 HC | QVQLVQSGAEVKKPGASVKVSCKASGYTFTSYYMHWVR |
| germline | QAPGQGLEWMGIINPSGGSTSYAQKFQGRVTMTRDTST |
| | STVYMELSSLRSEDTAVYYCAR |
| | (SEQ ID NO: 23) |
| Hu18B10-Ha | QVQLVQSGAEVKKPGASVKVSCKASGYTFTGYNMNWVR |
| | QAPGQGLEWMGNIDPYYGGTSYNQKFKGRVTMTIDKST |
| | STVYMELSSLRSEDTAVYYCARMYHGNAFDYWGQGTTV |
| | TVSS |
| | (SEQ ID NO: 25) |
| Hu18B10-Hb | QVQLVQSGAEVKKPGASVKVSCKASGYSFTGYNMNWVR |
| | QAPGQGLEWMGNIDPYYGGTSYNQKFKGRVTLTIDKST |
| | STVYMELSSLRSEDTAVYYCARMYHGNAFDYWGQGTTV |
| | TVSS |
| | (SEQ ID NO: 27) |
| Hu18B10-Hc | QVQLVQSGAEVKKPGASVKVSCKASGYSFTGYNMNWVK |
| | QAPGQGLEWIGNIDPYYGGTSYNQKFKGRVTLTIDKST |
| | STVYMELSSLRSEDTAVYYCARMYHGNAFDYWGQGTTV |
| | TVSS |
| | (SEQ ID NO: 29) |
| 18B10-LC | DIVMTQSPDSLAVSLGERATINCKSSQNNKNYLAWYQQK |
| germline | PGQPPKLLIYWASTRESGVPDRFSGSGSGTDFTLTISSL |
| | QAEDVAVYYCQQYYSTP |
| | (SEQ ID NO: 24) |
| Hu18B10_La | DIVMTQSPDSLAVSLGERATINCKSSQSLLNSGNLKNYL |
| | TWYQQKPGQPPKLLIYWASTRKSGVPDRFSGSGSGTDFT |
| | LTISSLQAEDVAVYYCQNDYSYPLTFGGGTKVEIK |
| | (SEQ ID NO: 26) |
| Hu18B10_Lb | DIVMTQSPDSLAVSLGERATMNCKSSQSLLNSGNLKNYL |
| | TWYQQKPGQPPKLLIYWASTRKSGVPDRFTGSGSGTDFT |
| | LTISSLQAEDVAVYYCQNDYSYPLTFGGGTKVEIK |
| | (SEQ ID NO: 28) |

In certain embodiments, the humanized antibodies provided herein may comprise the heavy chain variable region fused to the constant region of human IgG1 isotype and the light chain variable region fused to the constant region of human kappa chain.

The humanized anti-CLDN18.2 antibodies provided herein retained the specific binding affinity to CLDN18.2-expressing cell, and are at least comparable to, or even better than, the parent antibodies in that aspect. The humanized antibodies provided herein can also retain their functional interaction with CLDN18.2-expressing cells, such as NUGC4 cells, SNU-620 cell, SNU-601 cell, or KATOIII cell in that all antibodies can mediate cell killing by ADCC, CDC and induction of apoptosis induced by cross linking of the target at the tumor cell surface and direct inhibition of proliferation. In certain embodiments, the anti-CLDN18.2 antibodies and the fragments thereof provided herein further comprise an immunoglobulin constant region, optionally a constant region of human Ig, or optionally a constant region of human IgG. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

In certain embodiments, the anti-CLDN18.2 antibodies and the fragments thereof provided herein further comprise a constant region of human IgG1, IgG2, IgG3, or IgG4. In certain embodiments, the anti-CLDN18.2 antibodies and antigen-binding fragments thereof provided herein comprises a constant region of IgG1 isotype. In certain embodiments, the constant region of human IgG1 comprises SEQ ID NO: 49, or a homologous sequence having at least 80% (e.g. at least 85%, 90%, 95%, 96%, 97%, 98%, or 99%) sequence identity thereof.

Constant region of IgG1 isotype can induce effector functions such as ADCC or CDC. Effector functions of the anti-CLDN18.2 antibodies and the antigen-binding fragments thereof provided herein can lead to cytotoxicity to cells expressing CLDN18.2. Effector functions can be evaluated using various assays such as Fc receptor binding assay, C1q binding assay, and cell lysis assay, and any of the assays described above for determining ADCC or CDC.

Antibody Variants

The anti-CLDN18.2 antibodies and antigen-binding fragments thereof provided herein also encompass various types of variants of the antibody sequences provided herein.

In certain embodiments, the variants comprise one or more modification(s) or substitution(s) in 1, 2, or 3 CDR sequences as provided in Table 1, in one or more FR sequences, in the heavy or light chain variable region sequences provided herein, and/or in the constant region (e.g., Fc region). Such antibody variants retain specific binding affinity to CLDN18.2 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or increased effector function(s), improved FcRn receptor binding, increased pharmacokinetic half-life, pH sensitivity, and/or compatibility to conjugation (e.g., one or more introduced cysteine residues), to name a few.

A parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

1. Affinity Variant

An affinity variant retain specific binding affinity to CLDN18.2 of the parent antibody, or even have improved CLDN18.2 specific binding affinity over the parent antibody. Various methods known in the art can be used to achieve this purpose. For example, a library of antibody variants (such as Fab or scFv variants) can be generated and expressed with phage display technology, and then screened for the binding affinity to human CLDN18.2. For another example, computer software can be used to virtually simulate the binding of the antibodies to human CLDN18.2, and identify the amino acid residues on the antibodies which form the binding interface. Such residues may be either avoided in the substitution so as to prevent reduction in binding affinity, or targeted for substitution to provide for a stronger binding.

In certain embodiments, at least one (or all) of the substitution(s) in the CDR sequences, FR sequences, or variable region sequences comprises a conservative substitution. A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

In certain embodiments, the antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in one or more of the CDR sequences and/or FR sequences in total.

In certain embodiments, the anti-CLDN18.2 antibodies and antigen-binding fragments thereof comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Table 1, and in the meantime retain the binding affinity to CLDN18.2 at a level similar to or even higher than its parental antibody.

In certain embodiments, the anti-CLDN18.2 antibodies and antigen-binding fragments thereof comprise one or more variable region sequences having at least 80% (e.g., at least 85%, 88%, 90%, 91%, 92%, 93%, 94%, 95%, 96%, 97%, 98%, 99%) sequence identity to that (or those) of SEQ ID NOs: 23-29 and 37-48, and in the meantime retain the binding affinity to CLDN18.2 at a level similar to or even higher than its parent antibody. In some embodiments, a total of 1 to 10 amino acids have been substituted, inserted, or deleted in a sequence selected from SEQ ID NOs: 25-29 and 37-48. In some embodiments, the substitutions, insertions, or deletions occur in regions outside the CDRs (i.e., in the FRs).

2. Glycosylation Variant

The anti-CLDN18.2 antibodies and antigen-binding fragments provided herein also encompass a glycosylation variant, which can be obtained to either increase or decrease the extent of glycosylation of the antibody or antigen binding fragment. The term “glycosylation” as used herein, refers to enzymatic process that attaches glycans such as fucose, xylose, mannose, or GlcNAc phosphoserine glycan to proteins, lipids, or other organic molecules. Depending on the carbon linked to the glycan, glycosylation can be divided into five classes including: N-linked glycosylation, O-linked glycosylation, phospho-glycosylation, C-linked glycosylation, and glypiation.

Glycosylation of antibodies is typically N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine.

In certain embodiments, the anti-CLDN18.2 antibodies and antigen-binding fragments provided herein encompass a glycosylation variant having improved effector functions such as ADCC or CDC.

In certain embodiments, the antibody or antigen-binding fragment thereof provided herein is afucosylated. The term “afucosylation,” or “afucosylated,” refers to the reduced or eliminated core-fucose on the N-glycan attached to the antibody. The majority glycans of human IgG antibodies are known as G0, G1 and G2, which are complex biantennary molecules with core fucose residue carrying zero, one or two terminal galactose.

Afucosylated antibody variants can be made using methods known in the art, for example, as described in US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004).

In certain embodiments, the antibody glycosylation variant is afucosylated at Asn297 site of CH2 region in Fe of the antibody. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies.

In certain embodiments, the antibody glycosylation variants can be obtained by, for example, removal of a native glycosylation site (e.g. by N297A substitution), such that tripeptide sequences for N-linked glycosylation sites or serine or threonine residues for O-linked glycosylation sites no longer present in the antibody or Fc sequence. Alternatively, in certain embodiments, antibody glycosylation variants can be obtained by producing the antibody in a host cell line that is defective in adding the selected sugar group(s) to the mature core carbohydrate structure in the antibody.

3. Cysteine-Engineered Variant

The anti-CLDN18.2 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with, for example a cytotoxic and/or imaging compound, a label, or a radioisotope among others, at the site of the engineered cysteine, through for example a maleimide or haloacetyl. Methods for engineering antibodies or antigen-binding fragments to introduce free cysteine residues are known in the art, see, for example, WO2006/034488.

4. Fc Variants

The anti-CLDN18.2 antibodies and antigen-binding fragments provided herein also encompass an Fc variant, which comprises one or more amino acid residue modifications or substitutions at its Fc region and/or hinge region.

In certain embodiments, the anti-CLDN18.2 antibodies or antigen-binding fragments thereof comprise constant region comprising one or more amino acid residue substitutions or modifications conferring increased CDC or ADCC relative to wild-type constant region. Certain amino acid residues at CH2 domain of the Fc region can be substituted to provide for enhanced ADCC activity, for example, by enhancing the affinity of the Fc domain to FcγRIIIA. Methods of altering ADCC activity by antibody engineering have been described in the art, see for example, Shields R L. et al., J Biol Chem. 2001. 276(9): 6591-604; Idusogie E E. et al., J Immunol. 2000.164 (8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O, et al., Mol Cancer Ther. 2008, 7(8): 2517-27; Shields R. L. et al, J. Biol. Chem, 2002, 277: 26733-26740; Shinkawa T. et al, J. Biol. Chem, 2003, 278: 3466-3473.

In certain embodiments, the anti-CLDN18.2 antibodies or antigen-binding fragments comprise one or more amino acid substitution(s) that alters Complement Dependent Cytotoxicity (CDC), for example, by improving or diminishing C1q binding and/or Complement Dependent Cytotoxicity (CDC) (see, for example, WO99/51642; Duncan & Winter Nature 322:738-40 (1988); U.S. Pat. Nos. 5,648,260; 5,624,821; and WO94/29351 concerning other examples of Fc region variants.

In certain embodiments, the constant region of the antibodies or antigen-binding fragments thereof provided herein comprises one or more amino acid residue substitutions relative to SEQ ID NO: 49 (i.e. the wild-type sequence), selected from the group consisting of: L235V, F243L, R292P, Y300L, P396L, or any combination thereof. In certain embodiments, the constant region comprises the sequence of SEQ ID NO: 51.

In certain embodiments, the anti-CLDN18.2 antibodies or antigen-binding fragments comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved P K, published by Springer, 2010; Yeung, Y. et al, Cancer Research, 70: 3269-3277 (2010); and Hinton, P. et al, J. Immunology, 176:346-356 (2006).

Antigen-Binding Fragments

Provided herein are also anti-CLDN18.2 antigen-binding fragments. Various types of antigen-binding fragments are known in the art and can be developed based on the anti-CLDN18.2 antibodies provided herein, including for example, the exemplary antibodies whose CDR sequences are shown in Tables 1, and their different variants (such as affinity variants, glycosylation variants, Fc variants, cysteine-engineered variants and so on).

In certain embodiments, an anti-CLDN18.2 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g., for Fab, Fv and ScFv antibody fragments), screening from a phage display library as discussed above (e.g., for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck).

In certain embodiments, the anti-CLDN18.2 antibodies and antigen-binding fragments thereof provided herein are bivalent, tetravalent, hexavalent, or multivalent. The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

Bispecific Antibodies

In certain embodiments, the antibodies and antigen-binding fragments thereof provided herein are bispecific. The term “bispecific” as used herein encompasses molecules having more than two specificity and molecules having more than two specificity, i.e. multispecific. In certain embodiments, the bispecific antibodies and antigen-binding fragments thereof provided herein is capable of specifically binding to a first and a second epitopes of CLDN18.2, or capable of specifically binding to CLDN18.2 and a second antigen. In certain embodiments, the first epitope and the second epitopes of CLDN18.2 are distinct from each other or non-overlapping. In certain embodiments, the bispecific antibodies and antigen-binding fragments thereof can bind to both the first epitope and the second epitope at the same time. In certain embodiments, the second antigen is different from CLDN18.2.

In certain embodiments, the second antigen is an immune related target. In some embodiments, the bispecific antibodies and antigen-binding fragments thereof specifically bind to CLDN18.2 and an immune related target, and are capable of targeting the immune cells to CLDN18.2-expressing cells (e.g. CLDN18.2-expressing tumor cells), and/or activating CLDN18.2 specific immune response to the CLDN18.2-expressing target cells. An immune related target as used herein, encompasses a biological molecule that is involved in the generation or modulation of an immune response, optionally, cellular immune responses. An example of the immune related target is immune checkpoint molecule, and a surface molecule of a cytolytic immune cell such as T cell or natural killer (NK) cell.

Immune checkpoint molecule can mediate co-stimulatory signal to augment immune response, or can mediate co-inhibitory signals to suppress immune response. Examples of an immune checkpoint molecule include, for example, PD-L1, PD-L2, PD-1, CLTA-4, TIM-3, LAG3, A2AR, CD160, 2B4, TGF β, VISTA, BTLA, TIGIT, LAIR1, OX40, CD2, CD27, CD28, CD30, CD40, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-15, CD3, CD16 and CD83.

Cytolytic immune cells can be triggered by its surface molecule to attack and mediate lysis of a target cell such as a tumor cell. In certain embodiments, the second antigen is a T cell surface antigen. Examples of a T cell surface antigen include, without limitation, an antigen selected from the group consisting of CD3, CD2, CD4, CD5, CD6, CD8, CD28, CD40L and/or CD44, preferably CD3. In certain embodiments, said second antigen is the epsilon-chain of CD3. In certain embodiments, binding of said bispecific antibody to CD3 on T cells results in proliferation and/or activation of said T cells, which induces release of cytotoxic factors, e.g. perforins and granzymes, and cytolysis and apoptosis of the target cells. In certain embodiments, the second antigen is a NK cell surface antigen, such as CD16 (FcγRIII) or CD56. In certain embodiments, binding of bispecific antibody to CD16 on NK cells leads to NK-cell degranulation and perforin-dependent target cell lysis (ADCC) of the target cells.

In certain embodiments, the second antigen comprises a tumor antigen. “Tumor antigen” as used herein refers to tumor specific antigens (e.g. those unique to tumor cells and normally not found on non-tumor cells), tumor-associated antigens (e.g. found in both tumor and non-tumor cells but expressed differently in tumor cells), and tumor neo-antigens (e.g. that are expressed in cancer cells because of somatic mutations that change the protein sequence or create fusion proteins between two unrelated sequences).

Examples of tumor antigens include, without limitation, EpCAM, HER2/neu, HER3/neu, C250, CEA, MAGE, proteoglycans, VEGF, EGFR, αVβ-integrin, HLA, HLA-DR, ASC, CD1, CD2, CD4, CD6, CD7, CD8, CD11, CD13, CD14, CD19, CD20, CD21, CD22, CD23, CD24, CD30, CD33, CD37, CD40, CD41, CD47, CD52, c-erb-2, CALLA, MHCII, CD44v3, CD44v6, p97, ganglioside GM1, GM2, GM3, GD1a, GD1b, GD2, GD3, GT1b, GT3, GQ 1, NY-ESO-1, NFX2, SSX2, SSX4, Trp2, gp100 (Pmel 17), tyrosinase, Muc-1, telomerase, survivin, G250, p53, CA125 MUC, Wue antigen, Lewis Y antigen, HSP-27, HSP-70, HSP-72, HSP-90, Pgp, MCSP, EpHA2 and cell surface targets GC1 82, GT468 or GT512, PD-L1, arboviral E protein epitope, glioma-associated antigen, carcinoembryonic antigen (CEA), β-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxyi esterase, mut hsp70-2, M-CSF, prostase, prostate-specific antigen (PSA), PAP, NY-ESO-1, LAGE-la, p53, prostein, PSMA, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, ART-1/MelanA (MART-1), tyrosinase, TRP-1, TRP-2 and tumor-specific multilineage antigens such as MAGE-1, MAGE-3, BAGE, GAGE-1, GAGE-2, pi 5; Ras, unique tumor antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7; protein-based antigens include TSP-180, MAGE-4, MAGE-5, MAGE-6, RAGE, NY-ESO, pl 85erbB2, pl 80erbB-3, c-met, nm-23H1, PSA, TAG-72, CA19-9, CA72-4, CAM 17.1, NuMa, K-ras, beta-Catenin, CDK4, Mum-1, p15, p16, 43-9F, 5T4(791Tgp72), α-fetoprotem, beta-HCG, BCA225, BTAA, CA 125, CA 15-3\CA 27.29\BCAA, CA 195, CA 242, CA-50, CAM43, CD68\I, CO-029, FGF-5, G250, Ga733VEpCAM, HTgp-175, M344, MA-50, MG7-Ag, MOV 18, NB/70K, NY-CO-1, RCAS 1, SDCCAG16, TA-90\Mac-2 binding protein, cyclophilin C-associated protein, TAAL6, TAG72, TLP, and TPS.

In certain embodiments, the tumor antigen is associated with gastric cancer, esophageal cancer, pancreatic cancer, lung cancer, ovarian cancer, colon cancer, hepatic cancer, head-neck cancer, cancer of the gallbladder and the metastasis thereof. Examples of such tumor antigen include, but are not limited to, CA-125, gangliosides G (D2), G (M2) and G (D3), CD20, CD52, CD33, Ep-CAM, CEA, bombesin-like peptides, PSA, HER2/neu, epidermal growth factor receptor (EGFR), erbB2, erbB3/HER3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R or CO17-1A, CA19-9, and CA72-4. In certain embodiments, the tumor antigen is present in a CLDN18.2-expressing cell, for example, a CLDN18.2-expressing cancer cell.

Bispecific antibodies and antigen-binding fragments thereof provided herein can be in a suitable format known in the art. For example, an exemplary bispecific format can be, bispecific diabodies, scFv-based bispecific formats, IgG-scFv fusions, dual variable domain (DVD)-Ig, Quadroma, knobs-into-holes, common light chain (e.g., common light chain with knobs-into-holes, etc.), BiTE, CrossMab, CrossFab, Duobody, SEEDbody, leucine zipper, dual acting Fab (DAF)-IgG, and Mab² bispecific formats (see, e.g., Brinkmann et al. 2017, Mabs, 9(2): 182-212). The bispecific molecules can be in symmetric or asymmetric architecture.

The bispecific antibodies and antigen-binding fragments provided herein can be made with any suitable methods known in the art.

In one embodiment, two immunoglobulin heavy chain-light chain pairs having different antigenic specificities are co-expressed in a host cell to produce bispecific antibodies in a recombinant way (see, for example, Milstein and Cuello, Nature, 305: 537 (1983)), followed by purification by affinity chromatography.

In another embodiment, sequences encoding the antibody heavy chain variable domains for the two specificities are respectively fused to immunoglobulin constant domain sequences, followed by insertion to one or more expression vector(s) which is/are co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g., Gruber et al., J. Immunol., 152:5368 (1994).)

In another method, leucine zipper peptides from the Fos and Jun proteins can be linked to the Fab′ portions of two different antibodies by gene fusion. The linked antibodies are reduced at the hinge region to four half antibodies (i.e. monomers) and then re-oxidized to form heterodimers (Kostelny et al., J. Immunol., 148(5):1547-1553 (1992)).

The two antigen-binding domains may also be conjugated or cross-linked to form a bispecific antibody or antigen-binding fragment. For example, one antibody can be coupled to biotin while the other antibody to avidin, and the strong association between biotin and avidin would complex the two antibodies together to form a bispecific antibody (see, for example, U.S. Pat. No. 4,676,980; WO 91/00360, WO 92/00373, and EP 03089). For another example, the two antibodies or antigen-binding fragments can be cross-linked by conventional methods known in the art, for example, as disclosed in U.S. Pat. No. 4,676,980.

Bispecific antigen-binding fragments may be generated from a bispecific antibody, for example, by proteolytic cleavage, or by chemical linking. For example, an antigen-binding fragment (e.g., Fab′) of an antibody may be prepared and converted to Fab′-thiol derivative and then mixed and reacted with another converted Fab′ derivative having a different antigenic specificity to form a bispecific antigen-binding fragment (see, for example, Brennan et al., Science, 229: 81 (1985)).

In certain embodiments, the bispecific antibody or antigen-binding fragments thereof provided herein may be engineered at the interface so that a knob-into-hole association can be formed to promote heterodimerization of the two different antigen-binding sites. This can maximize the percentage of heterodimers which are recovered from recombinant cell culture. “Knob-into-hole” as used herein, refers to an interaction between two polypeptides (such as Fc), where one polypeptide has a protuberance (i.e. “knob”) due to presence of an amino acid residue having a bulky side chain (e.g., tyrosine or tryptophan), and the other polypeptide has a cavity (i.e. “hole”) where a small side chain amino acid residue resides (e.g., alanine or threonine), and the protuberance is positionable in the cavity so as to promote interaction of the two polypeptides to form a heterodimer or a complex. Methods of generating polypeptides with knobs-into-holes are known in the art, e.g., as described in U.S. Pat. No. 5,731,168.

Conjugates

In some embodiments, the anti-CLDN18.2 antibodies and antigen-binding fragments thereof are linked to one or more conjugate moieties. A conjugate is a moiety that can be attached to the antibody or antigen-binding fragment thereof. It is contemplated that a variety of conjugates may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In certain embodiments, the antibodies or antigen binding fragments thereof are linked to one or more conjugates via a linker. In certain embodiments, the linker is a hydrazone linker, a disulfide linker, a bifunctional linker, dipeptide linker, glucuronide linker, a thioether linker.

In certain embodiments, the anti-CLDN18.2 antibodies and antigen-binding fragments disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

The conjugate can be a clearance-modifying agent, therapeutic agent (e.g., a chemotherapeutic agent), a toxin, a radioactive isotope, a detectable label (e.g., a lanthanide, a luminescent label, a fluorescent label, or an enzyme-substrate label), a pharmacokinetic modifying moiety, a DNA-alkylators, a topoisomerase inhibitor, a tubulin-binders, other anticancer drugs, or a purifying moiety (such as a magnetic bead or nanoparticle).

Examples of detectable label may include a fluorescent labels (e.g., fluorescein, rhodamine, dansyl, phycoerythrin, or Texas Red), enzyme-substrate labels (e.g., horseradish peroxidase, alkaline phosphatase, luceriferases, glucoamylase, lysozyme, saccharide oxidases or β-D-galactosidase), radioisotopes, other lanthanides, luminescent labels, chromophoric moiety, digoxigenin, biotin/avidin, a DNA molecule or gold for detection.

Examples of radioisotopes may include ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P. Radioisotope labelled antibodies are useful in receptor targeted imaging experiments.

In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like.

In certain embodiments, the conjugate can be a purification moiety such as a magnetic bead or a nanoparticle.

Antibody-Drug Conjugates

In certain embodiments, the present disclosure provides antibody-drug conjugates (ADC) comprising any of the above anti-CLDN18.2 antibodies or antigen-binding fragments conjugated to a cytotoxic agent.

ADC can be useful for local delivery of cytotoxic agents, for example, in the treatment of cancer. This allows for targeted delivery of cytotoxic agents to tumors and intracellular accumulation therein, which is particularly useful where systemic administration of these unconjugated cytotoxic agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies ′84: Biological And Clinical Applications, A. Pinchera et al. (ed.s), pp. 475-506; Syrigos and Epenetos (1999) Anticancer Research 19:605-614; Niculescu-Duvaz and Springer (1997) Adv. Drg Del. Rev. 26:151-172; U.S. Pat. No. 4,975,278).

In certain embodiments, the cytotoxic agent can be any agent that is detrimental to cells or that can damage or kill cells. In certain embodiments, the cytotoxic agent is optionally a toxin, a chemotherapeutic agent (such as a DNA-alkylators, a topoisomerase inhibitor, a tubulin-binders, a growth inhibitory agent, or other anticancer drugs), or a radioactive isotope.

Examples of toxins include bacterial toxins and plant toxins, such as for example, diphtheria toxin, exotoxin A chain (from Pseudomonas aeruginosa), ricin, abrin, modeccin, alpha-sarcin, Aleurites fordii. proteins, dianthin proteins, Phytolaca americana proteins (PARI, PAPII, and PAP-S), Momordica charantia inhibitor, curcin, crotin, Sapaonaria officinalis inhibitor, gelonin, restrictocin, phenomycin, enomycin, and the tricothecenes (see, e.g., WO 93/21232). Such a large molecule toxin can be conjugated to the antibodies or antigen-binding fragments provided herein using methods known in the art, for example, as described in Vitetta et al (1987) Science, 238:1098.

The cytotoxic agent can also be small molecule toxins and chemotherapeutic agents, such as geldanamycin (Mandler et al (2000) Jour. of the Nat. Cancer Inst. 92(19):1573-1581; Mandler et al (2002) Bioconjugate Chem. 13:786-791), maytansine and maytansinoids (EP 1391213; Liu et al., (1996) Proc. Natl. Acad. Sci. USA 93:8618-8623; U.S. Pat. No. 5,208,020), calicheam icin (Lode et al (1998) Cancer Res. 58:2928; Hinman et al (1993) Cancer Res. 53:3336-3342), taxol, cytochalasin B, gramicidin D, ethidium bromide, emetine, mitomycin, etoposide, tenoposide, vincristine, vinblastine, vindesine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine), calicheamicin, maytansinoids, dolastatins, auristatins such as MMAE and MMAF (U.S. Pat. Nos. 5,635,483; 5,780,588), dolostatins, a trichothecene, and CC1065, and the derivatives thereof having cytotoxic activity.

The cytotoxic agent can also be a highly radioactive isotope. Examples include At²¹¹, I¹³¹, I¹²⁵, Y⁹⁰, Re¹⁸⁶, Sm¹⁵³, Bi²¹², P³², Pb²¹² and radioactive isotopes of Lu. Methods of conjugation of a radioisotope to an antibody is known in the art, for example, via a suitable ligand reagent (see, e.g., WO94/11026; Current Protocols in Immunology, Volumes 1 and 2, Coligen et al, Ed. Wiley-Interscience, New York, N.Y, Pubs. (1991)). A ligand reagent has a chelating ligand that can bind, chelate or otherwise complex a radioisotope metal, and also has a functional group that is reactive with a thiol of cysteine of an antibody or antigen-binding fragment. Exemplary chelating ligands include DOTA, DOTP, DOTMA, DTPA and TETA (Macrocyclics, Dallas, Tex.).

The cytotoxic agents can be linked to an antibody or antigen-binding fragment via any suitable linkers known in the art, see, for example, in U.S. Pat. Nos. 5,208,020, 6,441,163, or EP Patent 0 425 235 B1, Chari et al., Cancer Research 52:127-131 (1992), and US 2005/0169933 A1, the disclosures of which are hereby expressly incorporated by reference.

In certain embodiments, the linker is cleavable under a particular physiological environment, thereby facilitating release of the cytotoxic drug in the cell. For example, the linker can be an acid-labile linker, peptidase-sensitive linker, photolabile linker, dimethyl linker or disulfide-containing linker, thioether linker, and esterase labile linker (Chari et al., Cancer Research 52:127-131 (1992); U.S. Pat. No. 5,208,020). In some embodiments, the linker may comprise amino acid residues, such as a dipeptide, a tripeptide, a tetrapeptide or a pentapeptide. The amino acid residues in the linker may be natural or non-naturally occurring amino acid residues. Examples of such linkers include: valine-citrulline (ve or val-cit), alanine-phenylalanine (af or ala-phe), glycine-valine-citrulline (gly-yal-cit), glycine-glycine-glycine (gly-gly-gly), an valine-citrullin-p-aminobenzyloxycaronyl (“vc-PAB”). Amino acid linker components can be designed and optimized in their selectivity for enzymatic cleavage by a particular enzymes, for example, a tumor-associated protease, cathepsin B, C and D, or a plasmin protease.

In certain embodiments, the cytotoxic agents can be linked to the antibody or antigen-binding fragment thereof provided herein by a bifunctional linker reagent include, such as N-succinimidyl-3-(2-pyridyldithio) propionate (SPDP), succinimidyl-4-(N-maleimidomethyl)cyclohexane-1-carboxylate (SMCC), N-succinimidyl-4-(2-pyridylthio)pentanoate (SPP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), bis-active fluorine compounds (such as 1,5-difluom-2,4-dinitrobenzene), BMPS, EMCS, GMBS, HBVS, LC-SMCC, MBS, MPRH, SBAP, SIA, SIAB, SMPB, SMPH, sulfo-EMCS, sulfo-GMBS, sulfo-KMUS, sulfo-MBS, sulfo-SIAB, sulfo-SMCC, and sulfo-SMPB, and SVSG (succinimidyl-(4-vinylsulfone)benzoate). Those linker reagents are commercially available (e.g., from Pierce Biotechnology, Inc., Rockford, Ill., U.S.A, see pages 467-498, 2003-2004 Applications Handbook and Catalog).

In certain embodiments, in the ADC provided herein, an antibody (or antigen-binding fragment thereof) is conjugated to one or more cytotoxic agents at an antibody: agent ratio of about 1 to about 20, about 1 to about 6, about 2 to about 6, about 3 to about 6, about 2 to about 5, about 2 to about 4, or about 3 to about 4.

The ADC provided herein may be prepared by any suitable methods known in the art. In certain embodiments, a nucleophilic group of the antibody (or antigen-binding fragment thereof) is first reacted with a bifunctional linker reagent and then linked to the cytotoxic agent, or the other way around, i.e., first reacting a nucleophilic of the cytotoxic agent with a bifunctional linker and then linking to the antibody.

In certain embodiments, the cytotoxic agent may contain (or modified to contain) a thiol reactive functional group which may react with a cysteine thiol of a free cysteine of the antibodies or antigen-binding fragments provided herein. Exemplary thiol-reactive functional group include, for example, a maleimide, an iodoacetamide, a pyridyl disulfide, haloacetyl, succinimidyl ester (e.g., NHS, N-hydroxysuccinimide), isothiocyanate, sulfonyl chloride, 2,6-dichlorotriazinyl, pentafluorophenyl ester, or phosphoramidite (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671).

The cytotoxic agent or the antibody may react with a linking reagent before being conjugated to form the ADC. For example, N-hydroxysuccinimidyl ester (NHS) of a cytotoxic agent may be performed, isolated, purified, and/or characterized, or it may be formed in situ and reacted with a nucleophilic group of an antibody. Typically, the carboxyl form of the conjugate is activated by reacting with some combination of a carbodiimide reagent, e.g., dicyclohexylcarbodiimide; diisopropyl carbodiimide, or a uronium reagent, e.g., TsTu (O—(N-Succinimidyl)-N,N,N′,N′-tetramethyluronium tetrafluoroborate, HBTU (O-benzotriazol-1-yl)-N,N,N′N′-tetramethyluronium hexafluorophosphate), or HATU (O-(7-azabenzotriazol-1-yl)-N,N,N′,N′-tetramethyluronium hexafluorophosphate), an activator, such as 1-hydroxybenzotriazole (HOBt), and N-hydroxysuccinimide to give the NHS ester. In some cases, the cytotoxic agent and the antibody may be linked by in situ activation and reaction to form the ADC in one step. Other activating and linking reagents include TBTU (2-(1H-benzotriazo-1-yl)-1-1,3,3-tetramethyluronium hexafluorophosphate), TFFH (N,N′,N″,N′″-tetramethyluronium 2-fluoro-hexafluorophosphate), PyBOP (benzotriazole-1-yl-oxy-tris-pyrrolidino-phosphonium hexafluorophosphate, EEDQ (2-ethoxy-1-ethoxycarbonyl-1,2-dihydro-quinoline), DCC (dicyclohexylcarbodiimide); DIPCDI (diisopropylcarbodiimide), MSNT (1-(mesitylene-2-sulfonyl)-3-nitro-1H-1,2,4-triazole, and aryl sulfonyl halides, e.g., triisopropylbenzenesulfonyl chloride. In another example, the antibody or antigen-binding fragments may be conjugated to biotin, then indirectly conjugated to a second conjugate that is conjugated to avidin.

Chimeric Antigen Receptor (CAR) Composition

The present disclosure also provides chimeric antigen receptors (CARs) comprising an anti-CLDN18.2 antigen binding domain as provided herein and a T-cell activation domain. Chimeric antigen receptors (CARs) are engineered chimeric receptors that combine an antigen-binding domain of an antibody with one or more signaling domains for T cell activation. Immune cells such as T cells and Nature Killer (NK) cells can be genetically engineered to express CARs. T cells expressing a CAR are referred to as CAR-T cells. CAR can mediate antigen-specific cellular immune activity in the T cells, enabling the CAR-T cells to eliminate cells (e.g. tumor cells) expressing the targeted antigen. In one embodiment, binding of the CAR-T cells provided herein to CLDN18.2 expressed on cells such as cancer cells, results in proliferation and/or activation of said CAR-T cells, wherein said activated CAT-T cells can release cytotoxic factors, e.g. perforin, granzymes, and granulysin, and initiate cytolysis and/or apoptosis of the cancer cells.

In some embodiments, the T-cell activation domain of the CAR comprises a co-stimulatory signaling domain and a TCR signaling domain, which can be linked to each other in a random or in a specified order, optionally with a short peptide linker having a length of, for example, between 2 and 10 amino acids (e.g. glycine-serine doublet linker).

In some embodiment, the CAR further comprises a transmembrane domain. When expressed in cells, the anti-CLDN18.2 antigen binding domain is extracellular, and the T-cell activation domain is intracellular.

In certain embodiments, the CAR comprises an anti-CLDN18.2 antigen binding domain, a transmembrane domain, a costimulatory signaling region, and a TCR signaling domain, wherein the antigen binding domain specifically binds to CLDN18.2 and comprises an antigen-binding fragment of the antibodies provided herein.

1. Antigen Binding Domain

In some embodiments, the anti-CLDN18.2 antigen binding domain of the CAR comprises one or more CDR sequences as provided herein, one or more heavy chain variable domains or light chain variable domains provided herein, or one or more antigen-binding fragment derived from any of the anti-CLDN18.2 antibodies provided herein.

In some embodiments, it is beneficial for the antigen binding domain to be derived from the same species in which the CAR will ultimately be used in. For example, for use in humans, it may be beneficial to have the antigen binding domain used in the CAR derived from a human antibody or a humanized antibody. In some embodiments, the antigen binding domain comprises a single chain variable fragment (scFv). In some embodiment, the antigen binding domain may exist in a variety of other forms including, for example, Fv, Fab, and (Fab′)₂, as well as bi-functional (i.e. bi-specific) hybrid antibody fragments (e.g., Lanzavecchia et al., Eur. J. Immunol. 17, 105 (1987)). In certain embodiments, the antigen binding domain comprises a Fab or a scFv.

2. Transmembrane Domain

In certain embodiments, the CAR comprises a transmembrane domain fused to the extracellular antigen-binding domain of the CAR. In one embodiment, the transmembrane domain can be selected such that it is naturally associated with one of the domains in the CAR. In some instances, the transmembrane domain can be selected or modified to avoid binding to transmembrane domains of other members of the T cell receptor complex.

The transmembrane domain of the CAR provided herein may be derived from transmembrane domains of any natural membrane-bound or transmembrane protein, such as, for example, the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, and CD154. In some embodiments, the transmembrane domain of the CAR can also use a variety of human hinges such as human Ig (immunoglobulin) hinge.

Alternatively, the transmembrane domain of the CAR provided herein may be synthetic, for example, comprising predominantly hydrophobic residues such as leucine and valine. In one embodiment, a triplet of phenylalanine, tryptophan and valine is included at each end of a synthetic transmembrane domain. Optionally, a short oligo- or polypeptide linker, between 2 and 10 amino acids in length may form the linkage between the transmembrane domain and the intracellular signaling domain of the CAR. A glycine-serine doublet provides a particularly suitable linker.

3. TCR Signaling Domain

The T-cell activation domain of the CARs provided herein comprises a TCR signaling domain. The TCR signaling domain can activate the T cell which expresses the CAR, to exert at least one of the normal TCR effector functions of a T cell, for example, cytolytic activity or helper activity including the secretion of cytokines. The TCR signaling domain can be either full-length of a natural intracellular signal transduction domain, or a fragment thereof sufficient to transduce the TCR effector function signal.

Exemplary intracellular signaling domains useful in the CARs provided herein include, the cytoplasmic sequences of the T cell receptor (TCR) and co-receptors that act in concert to initiate signal transduction following antigen receptor engagement, as well as any derivative or variant of these sequences and any synthetic sequence that has the same functional capability.

The TCR signaling domain that acts in a stimulatory manner may contain signaling motifs which are known as immunoreceptor tyrosine-based activation motifs or ITAMs. Examples of ITAM containing TCR signaling domains useful in the CAR provided herein include those derived from TCR zeta, FcR gamma, FcR beta, CD3 μgamma, CD3 delta, CD3 epsilon, CD5, CD22, CD79a, CD79b, and CD66d. In certain embodiments, the TCR signaling domain comprises a cytoplasmic signaling sequence derived from CD3-zeta.

4. Co-Stimulatory Signaling Region

The T-cell activation domain of the CARs provided herein further comprises a co-stimulatory signaling region. Co-stimulatory signaling region acts in an antigen-independent manner to mediate TCR activation, and can be derived from a co-stimulatory molecule required for an efficient response of lymphocytes to an antigen. Exemplary co-stimulatory molecules include, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

5. Bispecific CAR

In certain embodiments, the CAR is bispecific. In certain embodiments, the bispecific CAR provided herein specifically binds to a first and a second epitope of CLDN18.2, or capable of specifically binding to CLDN18.2 and a second antigen.

In one embodiment said CAR binds to a native epitope of CLDN18.2 present on the surface of living cells.

6. Polynucleotide Sequence Encoding the CAR

In one aspect, the present disclosure further provides nucleic acid sequences encoding the CAR provided herein, comprising a first polynucleotide sequence encoding the antigen binding domain of the CAR provided herein, and optionally a second polynucleotide sequence encoding the transmembrane domain and the T-cell activation domain provided herein. In some embodiments, the sequence encoding the antigen binding domain is operably linked to the sequence encoding the transmembrane domain and the T-cell activation domain. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

In one aspect, the present disclosure provides vectors comprising the nucleic acid sequence encoding the CAR provided herein. In some embodiments, the vector is retroviral and lentiviral vector construct expressing the CAR of the present disclosure which can be directly transduced into a cell, or RNA construct that can be directly transfected into a cell.

In one aspect, the present disclosure provides isolated cells which comprises the nucleic acid sequence encoding the CAR and/or express the CAR provided herein.

In certain embodiments, the cell comprising the nucleic acid encoding the CAR or expressing the CAR is selected from the group consisting of a T cell, a NK cell, a cytotoxic T lymphocyte (CTL), and a regulatory T cell. In one embodiment, the cell comprising the nucleic acid encoding the CAR or expressing the CAR exhibits an antitumor immunity when the antigen binding domain of the CAR binds to its corresponding antigen. The cytotoxic lymphocytes will preferably be autologous cells, although heterologous cells or allogenic cells can be used. As used herein, “autologous” means any material derived from the same individual to whom it is later to be re-introduced into the individual.

In one aspect, the present disclosure further provides methods for stimulating a T cell-mediated immune response to a CLDN18.2-expressing cell or tissue in a subject, the method comprising administering to the subject an effective amount of a cell genetically modified to express the CAR provided herein.

In one aspect, the present disclosure further provides methods for treating a mammal having a disease, disorder or condition associated with an elevated expression of CLDN18.2, comprising administering to the mammal an effective amount of a cell genetically modified to express the CAR provided herein, thereby treating the mammal. In certain embodiments, the cell is an autologous T cell. In certain embodiments, the mammal has been diagnosed with the disease, disorder or condition associated with an elevated expression of CLDN18.2.

Polynucleotides and Recombinant Methods

The present disclosure provides isolated polynucleotides that encode the anti-CLDN18.2 antibodies and antigen-binding fragments thereof. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The present disclosure provides vectors (e.g. expression vectors) comprising the isolated polynucleotide provided herein. In certain embodiments, the expression vector provided herein comprises the polynucleotide encoding the antibodies or antigen-binding fragments thereof provided herein, at least one promoter (e.g. SV40, CMV, EF-1α) operably linked to the polynucleotide sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmids such as pcDNA3.3, pMID18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-CLDN18.2 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment provided herein are derived from multicellular organisms such as invertebrate cells, for example plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g. the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is a mammalian cultured cell line, such as CHO, BHK, NS0, 293 and their derivatives.

Host cells are transformed with the above-described expression or cloning vectors for anti-CLDN18.2 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. patent Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CLDN18.2 antibodies and antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

Composition

In another aspect, the present disclosure provides a composition comprising the anti-CLDN18.2 antibodies or antigen-binding fragments thereof.

In another aspect, the present disclosure provides a composition comprising the anti-CLDN18.2 antibodies or antigen-binding fragments thereof which are afucosylated. In certain embodiments, the anti-CLDN18.2 antibodies in the composition have an amount of fucose of 60% or less (e.g. less than 55%, 50%, 45%, 40%, 35%, 30%, 25%, 20%, 15% or 10%) of the total amount of oligosaccharides (sugars) at Asn297 according to the EU numbering system. The amount of fucose attached to the CH2 domain of the Fc region can be determined by calculating the average amount of fucose within the sugar chain at Asn297, relative to the sum of all glycostructures attached to Asn 297 (e.g. complex, hybrid and high mannose structures). The amount of fucose can be measured by methods known in the art, for example, by mass spectrometry. In an illustrative embodiment, antibody is treated by N-glycosidase (PNGaseF) to hydrolyze the N-sugar chain oligosaccharide from the antibody. The hydrolyzed oligosaccharide is labeled with the fluorescent marker RapiFluor-MS reagent, and separated by ultra-high-performance liquid-phase hydrophilic interaction chromatography and detected by a fluorescence detector (UPLC-HILIC-FLR). The area normalization method was used to calculate the proportion of various oligosaccharides. In another illustrative example, the amount of fucose can be measured by, MALDI-TOF mass spectrometry, as described in WO 2008/077546.

Pharmaceutical Composition

The present disclosure further provides pharmaceutical compositions comprising the anti-CLDN18.2 antibodies or antigen-binding fragments thereof (optionally afucosylated) and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxyanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcellulose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-CLDN18.2 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

Methods of Use

The present disclosure also provides therapeutic methods comprising: administering a therapeutically effective amount of the antibody or antigen-binding fragment as provided herein (optionally afucosylated) and/or the pharmaceutical composition provided herein to a subject in need thereof, thereby treating or preventing a CLDN18.2-related disease or condition.

In another aspect, methods are provided to treat a disease or condition in a subject that would benefit from modulation of CLDN18.2 activity, comprising administering a therapeutically effective amount of the antibody or antigen-binding fragment as provided herein (optionally afucosylated) and/or the pharmaceutical composition provided herein to a subject in need thereof. In certain embodiments, the disease or condition is a CLDN18.2 related disease or condition. In some embodiment, the CLDN18.2-related disease or condition is cancer.

In certain embodiments, the cancer is selected from gastric cancer, lung cancer, bronchial cancer, bone cancer, liver and bile duct cancer, pancreatic cancer, breast cancer, liver cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, and adenocarcinoma.

Examples of cancers include but are not limited to, non-small cell lung cancer (squamous/nonsquamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, lymphoid malignancy, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, classical Hodgkin lymphoma (CHL), primary mediastinal large B-cell lymphoma, T-cell/histiocyte-rich B-cell lymphoma, acute lymphocytic leukemia, acute myelocytic leukemia, acute myelogenous leukemia, chronic myelocytic (granulocytic) leukemia, chronic myelogenous leukemia, chronic lymphocytic leukemia, polycythemia vera, mast cell derived tumors, EBV-positive and -negative PTLD, and diffuse large B-cell lymphoma (DLBCL), plasmablastic lymphoma, extranodal NK/T-cell lymphoma, nasopharyngeal carcinoma, HHV8-associated primary effusion lymphoma, non-Hodgkin's lymphoma, multiple myeloma, Waldenstrom's macroglobulinemia, heavy chain disease, myelodysplastic syndrome, hairy cell leukemia and myelodysplasia, primary CNS lymphoma, spinal axis tumor, brain stem glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In certain embodiments, the cancer is a CLDN18.2-expressing cancer. “CLDN18.2-expressing cancer” as used herein refers to any cancer or tumor involving cancer cells expressing CLDN18.2.

In certain embodiments, the subject is identified as having a CLDN18.2-expressing cancer cell. The presence and/or expression level of CLDN18.2 on a cancer cell can be determined by various methods known in the art. A biological sample containing or suspected of containing a cancer cell can be obtained from the subject. In some embodiments, the biological sample can be derived from a cancer cell or cancer tissue, or tumor infiltrating immune cells. In certain embodiments, the biological sample may be further processed to, for example, isolate the analyte such as the nucleic acids or proteins. Presence and/or expression level of CLDN18.2 can be determined by, for example, quantitative fluorescence cytometry, immunohistochemistry (IHC), or nucleic acid based methods. For example, the biological sample from the subject can be exposed to anti-CLDN18.2 antibody or antigen-binding fragment thereof, which binds to and detects the expressed CLDN18.2 protein. Alternatively, CLDN18.2 can also be detected at nucleic acid expression level, using methods such as qPCR, reverse transcriptase PCR, microarray, SAGE, FISH, and the like.

In certain embodiments, the expression of CLDN18.2 in the biological sample or cancer cell is determined or measured by IHC. In certain embodiments, the expression level of human CLDN18.2 protein on a cancer cell from the subject can be determined in accordance to the methods described in section 6 and section 7 of Example 15 provided herein.

In certain embodiments, the subject is identified as having CLDN18.2 high-expressing cancer cells, CLDN18.2 medium-expressing cancer cells, or CLDN18.2 low-expressing cancer cells. In certain embodiments, the CLDN18.2 high-expressing cancer cells express CLDN18.2 at an intensity of at least 2+ as measured by IHC and at a level where at least 40% (e.g. at least 45%, at least 50%, at least 55%, at least 60%, at least 65%, at least 70%, at least 75%, at least 80%, at least 85%, at least 90%, at least 95%, 40-100%, 50-100%, 60-100%, 70-100%, 80-100%, 90-100%, 40-90%, 50-90%, 60-90%, 70-90%, 80-90%, 40-80%, 40-70%, 40-60%, 40-50%, 50-80%, 50-70%, 50-60%, 60-80%, 60-70%, or 70-80%) of the cells are stained positive in IHC; the medium-expressing cancer cells express CLDN18.2 at an intensity of at least 1+ and below 2+ as measured by IHC and at a level where at least 30% (or at least 35%) but below 40% of the cells are stained positive in IHC; and the low-expressing cancer cells express CLDN18.2 at an intensity of above 0 but below 1+ as measured by IHC and at a level where above 0 but below 30% (e.g. 5%, 10%, 15%, 20%, 25%, 5-25%, 10-25%, 15-25%, 20-25%, 5-20%, 5-15%, 5-10%, 10-20%, or 10-15%) of the cells are stained positive in IHC.

Examples of CLDN18.2-expressing cancer include, without limitation, gastric cancer, esophageal cancer, pancreatic cancer, lung cancer such as non-small cell lung cancer (NSCLC) and small cell lung cancer (SCLC), ovarian cancer, colon cancer, colorectal cancer, gastrointestinal stromal tumors (GIST), gastrointestinal carcinoid tumors, rectal cancer, anal cancer, bile duct cancer, small intestine cancer, appendix cancer; prostate cancer, renal cancer (e.g., renal cell carcinoma), hepatic cancer, head-neck cancer, and cancer of the gallbladder and metastases thereof, for example, gastric cancer metastasis such as Krukenberg tumors, peritoneal metastasis and lymph node metastasis.

In certain embodiments, the CLDN18.2-expressing cancer can be an adenocarcinoma, for example, an advanced adenocarcinoma. In certain embodiments, the cancer is selected from adenocarcinomas of the stomach, the esophagus, the pancreatic duct, the bile ducts, the lung and the ovary. In certain embodiments, the CLDN18.2-expressing cancer comprises a cancer of the stomach, a cancer of the esophagus, in particular the lower esophagus, a cancer of the eso-gastric junction and gastroesophageal cancer.

Without wishing to be bound to any theories, it is believed that the molecular and functional characteristics of CLDN18 make it a highly interesting target for antibody-based cancer therapy. These include (i) absence of CLDN18 from the majority of toxicity relevant normal tissues, (ii) restriction of CLDN18.2 variant expression to a dispensable cell population as differentiated gastric cells that can be replenished by target-negative stem cells of the stomach, (iii) potential differential glycosylation between normal and neoplastic cells, and (iv) the presence of different conformational topologies.

It has been found that the molecular weight of the CLDN18 protein differs between tumors and adjacent normal tissues. The higher molecular weight CLDN18 protein is observed in healthy tissues, which can be decreased to the same molecular weight as observed in tumor by treatment of the normal tissue lysates with deglycosylating compound PNGase F. This suggests that CLDN18 is less N-glycosylated in tumor as compared to its normal tissue counterpart. A classical N-glycosylation motif is in amino acid residue 116 within the loop D3 domain of the CLDN18 molecule. The molecular weight difference and the inferred structural difference may represent an altered epitope for antibody binding.

In addition, CLDN18 as a tight junction protein may also contribute to a good therapeutic window. Since tumor cells express CLDNs but often do not form the classical tight junctions by homotypic and heterotypic association of CLDNs as found in normal epithelial tissue, they likely have a considerable pool of free CLDNs that are amenable to extracellular antibody binding and immunotherapy. It is possible that binding epitopes of CLDNs in healthy epithelium are shielded within the tight junctions from being accessed to antibody binding.

The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the antibody or antigen-binding fragment as provided herein may be administered at a therapeutically effective dosage of about 0.01 mg/kg to about 100 mg/kg. In certain embodiments, the administration dosage may change over the course of treatment. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments disclosed herein may be administered alone or in combination with one or more additional therapeutic means or agents. For example, the antibodies or antigen-binding fragments disclosed herein may be administered in combination with a second therapeutic agent, for example, a chemotherapeutic agent, an anti-cancer drug, radiation therapy, an immunotherapy, anti-angiogenesis agent, a targeted therapy, a cellular therapy, a gene therapy, a hormonal therapy, palliative care, surgery for the treatment of cancer (e.g., tumorectomy), or one or more anti-emetics or other treatments for complications arising from chemotherapy

The term “immunotherapy” as used herein, refers to a type of that stimulates immune system to fight against disease such as cancer or that boosts immune system in a general way. Immunotherapy includes passive immunotherapy by delivering agents with established tumor-immune reactivity (such as effector cells) that can directly or indirectly mediate anti-tumor effects and does not necessarily depend on an intact host immune system (such as an antibody therapy or CAR-T cell therapy). Immunotherapy can further include active immunotherapy, in which treatment relies on the in vivo stimulation of the endogenous host immune system to react against diseased cells with the administration of immune response-modifying agents.

Examples of immunotherapy include, without limitation, checkpoint modulators, adoptive cell transfer, cytokines, oncolytic virus and therapeutic vaccines.

Checkpoint modulators can interfere with the ability of cancer cells to avoid immune system attack, and help the immune system respond more strongly to a tumor. Immune checkpoint molecule can mediate co-stimulatory signal to augment immune response, or can mediate co-inhibitory signals to suppress immune response. Examples of checkpoint modulators include, without limitation, modulators of PD-1, PD-L1, PD-L2, CLTA-4, TIM-3, LAG3, A2AR, CD160, 2B4, TGF β, VISTA, BTLA, TIGIT, LAIR1, OX40, CD2, CD27, CD28, CD30, CD40, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-15, CD3, CD16 and CD83.

Adoptive cell transfer, which is a treatment that attempts to boost the natural ability of the T cells to fight cancer. In this treatment, T cells are taken from the patient, and are expanded and activated in vitro. In certain embodiments, the T cells are modified in vitro to CAR-T cells. T cells or CAR-T cells that are most active against the cancer are cultured in large batches in vitro for 2 to 8 weeks. During this period, the patients will receive treatments such as chemotherapy and radiation therapy to reduce the body's immunity. After these treatments, the in vitro cultured T cells or CAR-T cells will be given back to the patient. In certain embodiments, the immunotherapy is CAR-T therapy.

Cytokine therapy can also be used to enhance tumor antigen presentation to the immune system. The two main types of cytokines used to treat cancer are interferons and interleukins. Examples of cytokine therapy include, without limitation, interferons such as interferon-α, -β, and -γ, colony stimulating factors such as macrophage-CSF, granulocyte macrophage CSF, and granulocyte-CSF, insulin growth factor (IGF-1), vascular endothelial growth factor (VEGF), transforming growth factor (TGF), fibroblast growth factor (FGF), interleukins such as IL-1, IL-la, IL-2, IL-3, IL-4, IL-5, IL-6, IL-7, IL-8, IL-9, IL-10, IL-11, and IL-12, tumor necrosis factors such as TNF-α and TNF-β or any combination thereof.

Oncolytic virus are genetically modified virus that can kill cancer cells. Oncolytic virus can specifically infect tumor cells, thereby leading to tumor cell lysis followed by release of large amount of tumor antigens that trigger the immune system to target and eliminate cancer cells having such tumor antigens. Examples of oncolytic virus include, without limitation, talimogene laherparepvec.

Therapeutic vaccines work against cancer by boosting the immune system's response to cancer cells. Therapeutic vaccines can comprise non-pathogenic microorganism (e.g. Mycobacterium bovis Bacillus Calmette-Guerin, BCG), genetically modified virus targeting a tumor cell, or one or more immunogenic components. For example, BCG can be inserted directly into the bladder with a catheter and can cause an immune response against bladder cancer cells.

Anti-angiogenesis agent can block the growth of blood vessels that support tumor growth. Some of the anti-angiogenesis agent target VEGF or its receptor VEGFR. Examples of Anti-angiogenesis agent include, without limitation, Axitinib, Bevacizumab, Cabozantinib, Everolimus, Lenalidomide, Lenvatinib mesylate, Pazopanib, Ramucirumab, Regorafenib, Sorafenib, Sunitinib, Thalidomide, Vandetanib, and Ziv-aflibercept.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

Targeted therapy can target, for example, tyrosine kinase receptors and nuclear receptors. Examples of such receptors include, erbB1 (EGFR or HER1), erbB2 (HER2), erbB3, erbB4, FGFR, platelet-derived growth factor receptor (PDGFR), and insulin-like growth factor-1 receptor (IGF-1R), estrogen receptors (ERs), nuclear receptors (NR) and PRs.

Targeted therapy can target molecules in tyrosine kinase or nuclear receptors signaling cascade, such as, Erk and PI3K/Akt, AP-2α, AP-2β, AP-2γ, mitogen-activated protein kinase (MAPK), PTEN, p53, p19ARF, Rb, Apaf-1, CD-95/Fas, TRAIL-R1/R2, Caspase-8, Forkhead, Box 03A, MDM2, IAPs, NF-kB, Myc, P13K, Ras, FLIP, heregulin (HRG) (also known as gp30), Bcl-2, Bcl-xL, Bax, Bak, Bad, Bok, Bik, Blk, Hrk, BNIP3, BimL, Bid, and EGL-1.

Targeted therapy can also target tumor-associated ligands such estrogen, estradiol (E2), progesterone, oestrogen, androgen, glucocorticoid, prolactin, thyroid hormone, insulin, P70 S6 kinase protein (PS6), Survivin, fibroblast growth factors (FGFs), EGF, Neu Differentiation Factor (NDF), transforming growth factor alpha (TGF-α), IL-1A, TGF-beta, IGF-1, IGF-II, IGFBPs, IGFBP proteases, and IL-10.

In certain of these embodiments, an antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides methods of using the anti-CLDN18.2 antibodies or antigen-binding fragments thereof. In some embodiments, the present disclosure provides methods of inhibiting growth of CLDN18.2-expressing cells in vivo or in vitro, comprising: contacting the CLDN18.2-expressing cells with the antibody or antigen-binding fragment thereof provided herein. In some embodiments, the present disclosure provides methods of modulating CLDN18.2 activity in a CLDN18.2-expressing cell, comprising exposing the CLDN18.2-expressing cell to the antibody or antigen-binding fragment thereof provided herein.

In some embodiments, the present disclosure provides methods of detecting presence or amount of CLDN18.2 in a sample derived from a subject, comprising contacting the sample with the antibody or antigen-binding fragment thereof, and determining the presence or the amount of CLDN18.2 in the sample. In certain embodiments, the biological sample comprises a cancer cell.

In some embodiments, the present disclosure provides methods of diagnosing a CLDN18.2 related disease or condition in a subject, comprising: a) contacting a sample obtained from the subject with the antibody or antigen-binding fragment thereof provided herein; b) determining presence or amount of CLDN18.2 in the sample; and c) correlating the presence or the amount of CLDN18.2 to existence or status of the CLDN18.2 related disease or condition in the subject. In certain embodiments, the biological sample comprises a cancer cell. In some embodiments, the expression level of CLDN18.2 in the cancer cell is determined by IHC (for example, in accordance to the methods described in section 6 and section 7 of Example 15 provided herein). In some embodiments, the subject is identified as having a CLDN18.2 high-expressing cancer cell, a CLDN18.2 medium-expressing cancer cell, or a CLDN18.2 low-expressing cancer cell.

In some embodiments, the method further comprises administering a therapeutically effective amount of the antibody or antigen-binding fragment thereof provided herein to the subject. In some embodiments, the subject is as having a CLDN18.2 medium-expressing cancer cell, or a CLDN18.2 low-expressing cancer cell.

In some embodiments, the present disclosure provides kits comprising the antibody or antigen-binding fragment thereof provided herein, optionally conjugated with a detectable moiety. The kits may be useful in detection of presence or amount of CLDN18.2 in a biological sample, or may be useful in the methods of diagnosis provided herein.

In some embodiments, the present disclosure provides kits comprising the antibody or antigen-binding fragment thereof provided herein and a second therapeutic agent. The kits may be useful in treatment, prevention, and/or amelioration of CLDN18.2 related disease.

In some embodiments, the present disclosure also provides use of the antibody or antigen-binding fragment thereof provided herein in the manufacture of a medicament for treating a CLDN18.2 related disease or condition in a subject.

### EXAMPLES

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1: Preparation of CLDN18.2 or CLDN18.1 Expressing Cell Lines

1. Generation of HEK293-Human CLDN18.2, HEK293-Human CLDN18.1 and HEK293-Mouse CLDN18.2 Cell Lines

HEK293-human CLDN18.2 cell (hereafter referred as HEK293-CLDN18.2) and HEK293-mouse CLDN18.2 cell (hereafter referred as HEK293-mCLDN18.2) were constructed by MabSpace Biosciences (Suzhou) Co., Limited. Briefly, HEK293 cell (Shanghai Institutes for Biological Sciences, Cat #GNhu43) was transfected with pcDNA3.1/hCLDN18.2 or pcDNA3.1/mCLDN18.2 plasmids, and selected with G418 to obtain stable expressing cell line HEK293-CLDN18.2 or HEK293-mCLDN18.2. The expression level of hCLDN18.2 or mCLDN18.2 was detected by IMAB362 antibody, which can bind to both human and mouse CLDN18.2. IMAB362 was expressed it according to the sequence disclosed in US2009169547A1. The single cell clone with a highest signal was selected and amplified for cell banking.

Heavy chain variable region of IMAB362
(SEQ ID NO: 72)
QVQLQQPGAELVRPGASVKLSCKASGYTFTSYWINWVKQRPGQGLEWIG
NIYPSDSYTNYNQKFKDKATLTVDKSSSTAYMQLSSPTSEDSAVYYCTR
SWRGNSFDYWGQGTTLTVSS
Light chain variable region of IMAB362
(SEQ ID NO: 73)
DIVMTQSPSSLTVTAGEKVTMSCKSSQSLLNSGNQKNYLTWYQQKPGQP
PKLLIYWASTRESGVPDRFTGSGSGTDFTLTISSVQAEDLAVYYCQNDY
SYPFTFGSGTKLEIK

HEK293-human CLDN18.1 cells (hereafter referred as HEK293-CLDN18.1) were also constructed as above. The expression of CLDN18.1 was detected by anti-CLDN18 antibody (Abcam, Cat #ab222513), which recognizes both CLDN18.1 and CLDN18.2.

2. Generation of CHO-CLDN18.2 Transient Expressing Cell

CHO-CLDN18.2 expressing cell was constructed as following: CHO cells were transiently transfected with pcDNA3.1/CLDN18.2 without selection reagent. Cell membrane protein was extracted using Mem-PER™ Plus Membrane Protein Extraction Kit and used for animal immunization boost.

3. Generation of MKN45-CLDN18.2 Transient Expressing Cell

MKN45-CLDN18.2 cell was constructed by MabSpace Biosciences (Suzhou) Co., Limited. Briefly, MKN45 cell (National Infrastructure of Cell Line Resource, Cat #3111C0001CCC000229) was transfected with pcDNA3.1/CLDN18.2 plasmids, and selected with G418 to obtain stable expressing cell line MKN45-CLDN18.2. The expression level of CLDN18.2 was detected by IMAB362 antibody using FACS method. The monoclonal cells with a highest, medium and low signal were selected and amplified for cell banking.

The above cell lines were used in the following experiments.

### Example 2: Antibody Generation

1. Immunization

Both DNA and cell immunogen were prepared for immunization. 6-8 weeks different strains of mice were divided into 2 μgroups. One is initiated and boosted with i.v. injection with 100 μg/mouse pVAC2-mcs/CLDN18.2 plasmid and 100 μg/mouse CpG. The other is i.m. injection with same DNA and CpG. Both groups are injected on Day 1 and Day 10 and the antibody titer was detected on Day 18 by FACS binding to HEK293-CLDN18.2 cell. 100 μl/well diluted mouse serum was added into a plate containing HEK293-CLDN18.2 or gastric cancer NUGC4 cells (JCRB, Cat #JCRBB0834), and then incubated at 4° C. for 30 min. After washing with buffer, 100 μl/well goat anti-mIgG-FITC (1:500 dilution) was added for another incubation at 4° C. for 30 min. Followed by washing with FACS washing buffer, cells were analyzed by Flow Cytometry. Mice with the higher binding signal and titer were selected for the following fusion procedures.

2. Fusions

Four days prior to fusion, each mouse was boosted intraperitoneally with 5×10\hat{ }7 HEK293-CLDN18.2 cells. On the fusion day, the spleens were removed aseptically and then processed into a single cell suspension. Viable, log-phase myeloma cells (SP2/0) were mixed with the murine splenocytes in a 1:1 ratio in a fusion medium followed by electrofusion for 1 min. Cells were resuspended and cultured in 96-well culture plates at 200 μl/well at a 37° C., 5% CO₂ incubator. After 7 days' culture, the growth media was exchanged for fresh growth media, followed by screening of hybridoma supernatants after 2-3 days.

### Example 3: Antibody Screening

1. Screening for Human CLDN18.2 Positive Binders by a FACS Assay

Log-phase CLDN18.2 expressing HEK293-CLDN18.2 cells were resuspended in PBS at a density of 10\hat{ }5/100 μl per well. After 3× cell wash by using FACS washing buffer (PBS+2% FBS), 100 μl/well hybridoma supernatant was added into each well for incubation at 4° C. for 30 min. Again, cells were washed 3 times by using FACS washing buffer and then incubated with 100 μl/well goat anti-mIgG-FITC (1:400 dilution) at 4° C. for another 30 min. After a final 3× wash using FACS washing buffer, cells were analyzed by flow Cytometry.

2. Screening for CLDN18.1 Negative Binders by a FACS Assay

Log-phase CLDN18.1 expressing HEK293-CLDN18.1 cells were resuspended in PBS at a density of 10\hat{ }5/100 μl per well. After 3× cell wash by using FACS washing buffer (PBS+2% FBS), 100 μl/well hybridoma supernatant was added into each well for incubation at 4° C. for 30 min. Again, cells were washed 3 times by using FACS washing buffer and then incubated with 100 μl/well goat anti-mIgG-FITC (1:400 dilution) at 4° C. for another 30 min. After a final 3× wash using FACS washing buffer, cells were analyzed by flow Cytometry.

The clones with a high signal of CLDN18.2 binding but no binding of CLDN18.1 were selected for subsequent subcloning to generate mono clones, including 7C12, 11F12, 12E9, 26G6, 59A9, 18B10, and 12C12.

### Example 4: Subcloning of the Positive Hybridoma Clones and Small-Scale Antibody Production

1. Subcloning of the Positive Hybridoma Clones

Cells from the FACS positive hybridoma wells with the desired binding profile were selected for a limited dilution in 96-well plates. These cells were allowed to grow for 7 days. Upon adequate cell mass was reached, supernatant from each well was collected and re-screened by using a cell binding assay (see Example 3).

From each 96-well plate, the clone with a highest cell binding activity was expanded for 2nd round limited dilution into a 96-well plate with 200 μl of hybridoma growth medium per well. After 7 days, supernatant of cells from the 96-well plates were analyzed by a FACS assay. The subcloning was done more than 2 times until more than 90/96 wells display a positive binding signal. Clones with the highest binding activity were identified and further expanded and cultured for antibody production. Isotypes were determined using a standard method.

2. Small-Scale Antibody Production

Hybridoma cells were inoculated and cultured for 14 days. CLDN18.2 monoclonal antibodies (mAbs) were purified from the hybridoma cell culture by affinity chromatography using Protein A chromatography column (Protein A High Performance (Bio-Rad)).

After purification, the CLDN18.2 mAbs were formulated in PBS by dialysis using 10,000 MWCO membranes (Pierce Slide-A-Lyzer or dialysis tubing), followed by a step of filtration.

### Example 5: Cell Binding Analysis of the Purified CLDN18.2 Hybridoma Antibodies

Log-phase HEK293-CLDN18.2 and NUGC4 cells were re-suspended in PBS. After 3× cell wash by using FACS washing buffer (PBS+2% FBS), 100 μl/well diluted hybridoma Abs with a range from 400 nM to 0.002 nM were added into each well for incubation at 4° C. for 30 min. Again, cells were washed 3 times by using FACS washing buffer and then incubated with 100 μl/well goat anti-mIgG-FITC (1:400 dilution) at 4° C. for another 30 min. After a final 3× wash using FACS washing buffer, cells were analyzed by flow Cytometry.

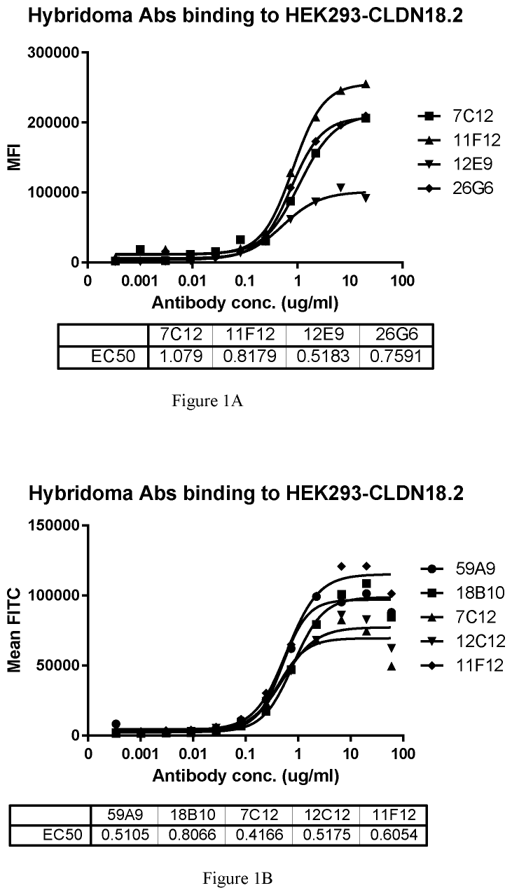

Most of the hybridoma antibodies showed a high affinity binding to HEK293-CLDN18.2 cells, but a less binding to NUGC4 cells. The binding difference is likely due to the different expression density, conformation and/or glycosylation status of CLDN18.2 protein in these two cell lines. Interestingly, 7C12, 11F12, 59A9 and 18B10 had comparable binding affinities to both HEK293-CLDN18.2 and NUGC4 cells (FIG. 1A-1D, Table 4). These hybridoma antibodies are selected for gene cloning and chimeric antibody expression for further functional ADCC/CDC characterization.

TABLE 4
EC50 values of CLDN18.2-specific antibodies
obtained in FACS binding (μg/ml)

| Hybridoma antibodies | HEK293-CLDN18.2 cell |
|---|---|
| 7C12 | 1.08 |
| 11F12 | 0.82 |
| 12E9 | 0.52 |
| 26G6 | 0.76 |
| 18B10 | 0.81 |
| 59A9 | 0.51 |

### Example 6: Generation of Chimeric Antibodies

The sequences of mouse anti-human CLDN18.2 antibody light chain and heavy chain variable regions were obtained by the polymerase chain reaction (PCR) amplification from the candidate hybridoma cell lines. After sequencing analysis and confirmation, the above variable region genes, including the sequence of the light chain variable region (VL) fused to human IgG kappa constant region and the sequence of the heavy chain variable region (VH) fused to human IgG1 constant region, were cloned into a recombinant expression vector, pcDNA3.1(+), for antibody production and purification.

ExpiCHO cells were transfected by using ExpiCHO transfection kit with an equal amount of DNA from the heavy chain vector and the light chain vector. The transfected cells were cultured in shake flasks at 125 rpm in 8% C02 and 37° C. incubator. Cell Culture was harvested on day 10, and the harvested antibodies were purified by affinity chromatography. The resulting antibody was analyzed to determine the level of purity using SDS-PAGE and size exclusion chromatography (TSKgel G3000SWXL, TOSOH). The chimeric antibodies were designated as: 7C12-C, 11F12-C, 12E9-C, 26G6-C, 59A9-C, 18B10-C, and 12C12-C.

### Example 7: Characterization of Purified Chimeric CLDN18.2 Antibodies

1. Binding and Cytotoxic Effect on HEK293-CLDN18.2 Cell

Cell binding of the chimeric antibodies was detected following the method described in Example 5.

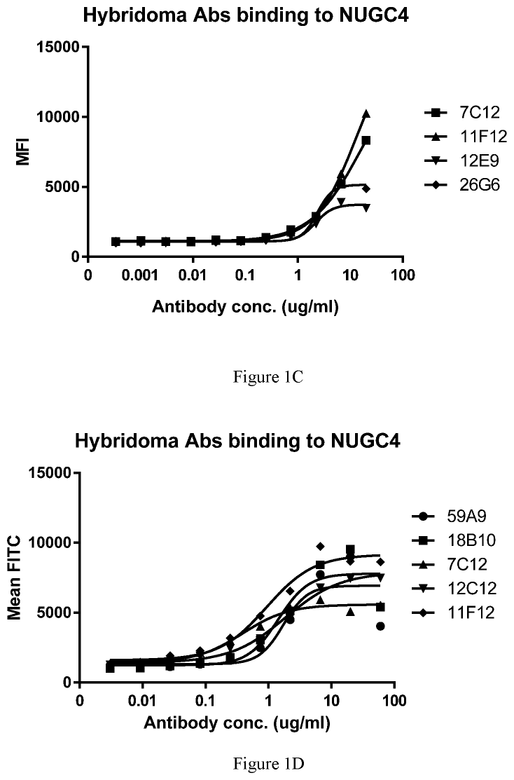

As showing in FIG. 2A, 7C12-C, 11F12-C and 12E9-C, which had a very similar CDR (only 2-3 amino acids different), bound to HEK293-CLDN18.2 cell with the EC50 around 0.6 μg/ml. 26G6-C had an EC50 of 1.1 μg/ml. 59A9-C and 18B10-C were produced later, so that they were tested separately. As shown in FIG. 2C, 59A9-C, with a different germline and CDR, had a slightly higher EC50 (1.3 μg/ml) than 18B10-C (1.0 μg/ml).

CDC (complement dependent cytotoxicity) was an important mechanism of immune protection. Therefore, CDC assay was used here for evaluation of antibody biological potency. Briefly, log-phase HEK293-CLDN18.2 cells were resuspended in RPMI1640 with 10% FBS. These cells were plated at 8×10\hat{ }3/100 μl per well. Anti-CLDN18.2 chimeric antibodies and the control antibody IMAB362 were diluted by using 60% RPMI1640 with 20 mM HEPES and 40% human serum, and then added into the cell plate at a final concentration from 10 to 0.0012 μg/ml, 100 μl/well. Plates were incubated at 37° C. for 80 min. Next, the cell culture plates were allowed to equilibrate to room temperature for 30 minutes. The CellTiter-Glo Luminescent Cell Viability Assay Kit was used for cell viability analysis at a room temperature by using the microplate reader (Thermo VARIOSKAN FLASH 3001).

As shown in FIGS. 2B and 2D, all 6 CLDN18.2 chimeric antibodies induced CDC effect at a lower concentration as compared to IMAB362. The potency of 4 antibodies (7C12-C, 11F12-C, 12E9-C and 26G6-C) were over 2-fold increase than IMAB362. 59A9-C and 18B10-C had over 3-fold increase in potency as compared to IMAB362.

2. Binding and Cytotoxic Effect on MKN45-CLDN18.2 Cell

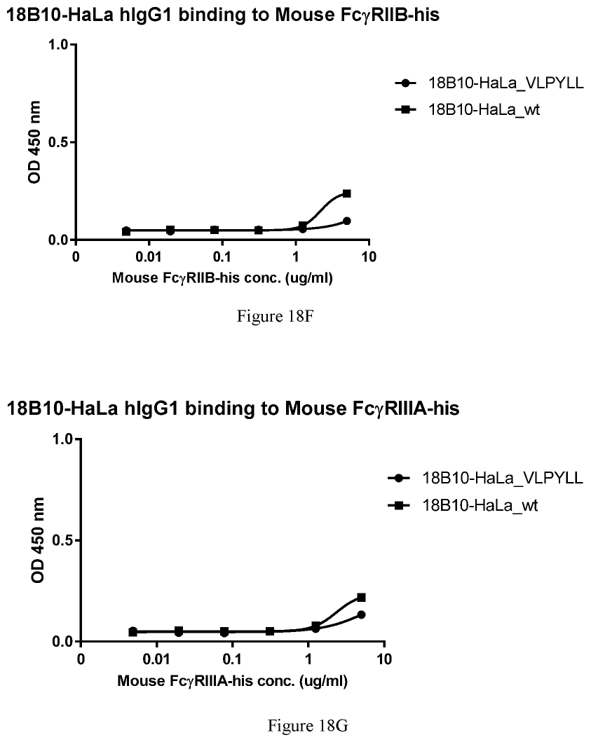

MKN45 is a poorly differentiated gastric adenocarcinoma and suitable for evaluating anti-tumor efficacy in vivo. However, MKN45 cell does not express human CLDN18.2 unless transfection. We found that different expression level of human CLDN18.2 on MKN45 cell conferred different sensitivity to the CLDN18.2 antibodies. Next, high and medium CLDN18.2 expressing MKN45 cells (see FIG. 21) were selected for the following study.

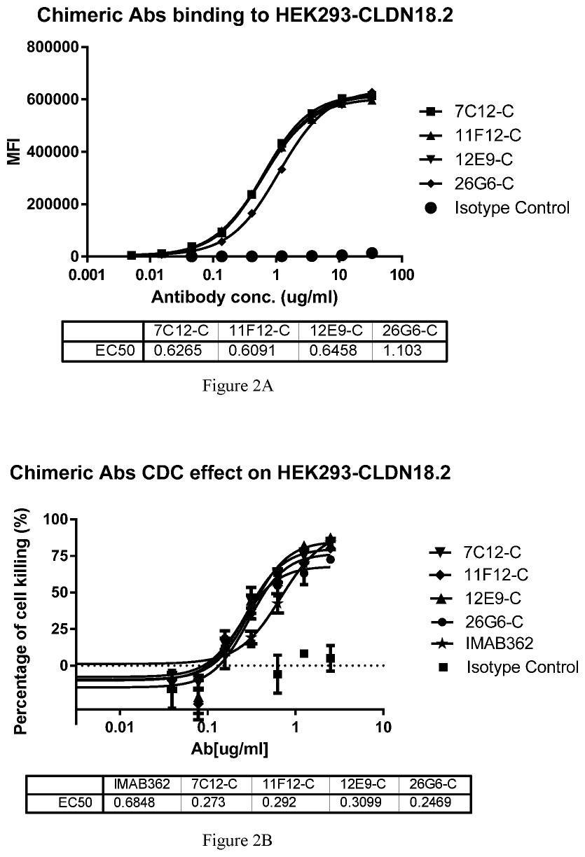

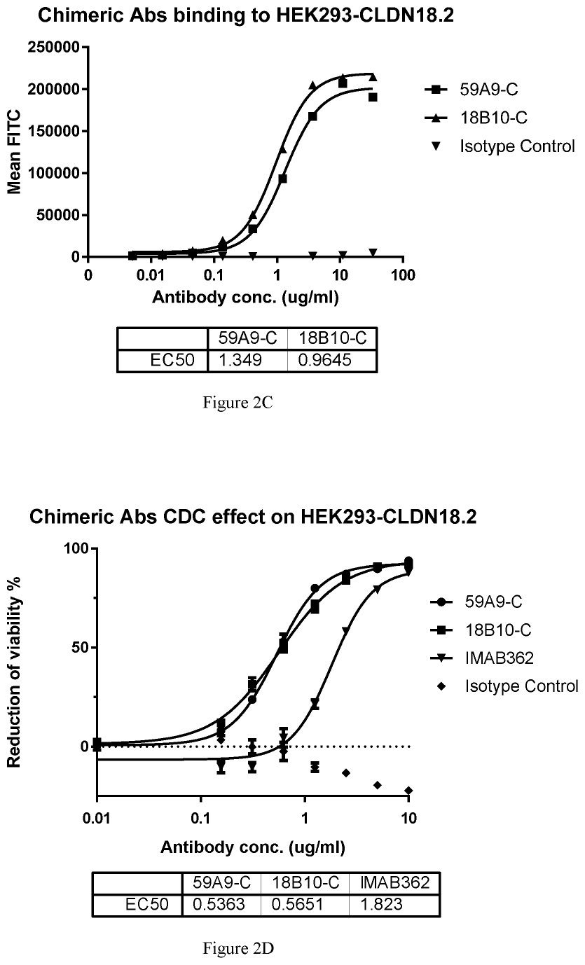

Cell binding assay of the chimeric antibodies was performed as described in Example 5, using the high and medium CLDN18.2 expressing MKN45 cells. As shown in FIGS. 3A (high) and 3C (medium), 18B10-C bound to both high and medium hCLDN18.2 expressing cells with a significant higher affinity than IMAB362. In MKN45-CLDN18.2-high cell, potency of 18B10-C was about 2-fold increase than IMAB362. A much more significant difference was seen in MKN45-CLDN18.2-medium cell, 18B10-C showed the EC50 at 0.96 μg/ml while IMAB362 had no binding.

ADCC activity was evaluated by using Jurkat-NFAT-luc-FcγRIIIA-V176 cells as effector cells and MKN45-CLDN18.2 cells as target cells. Jurkat-NFAT-luc-FcγRIIIA-V176 cell was constructed at Mabspace Biosciences (Suzhou) Co., Limited. Briefly, Jurkat cell (Shanghai Institutes for Biological Sciences, Cat #SCSP-513) was transfected with pGL4.30-luc/NFAT-RE/Hygro plasmids, and the selected with hygromycin to obtain the stable expressing cell line Jurkat-NFAT-luc. The Jurkat-NFAT-luc cell line was further transfected with pcDNA3.1-FcγRIIIA-V176 plasmids, and selected with antibiotic G418 to obtain the stable expression cell line Jurkat-NFAT-luc-FcγRIIIA-V176.

Next, log-phase target cells were re-suspended in RPMI1640 with 10% FBS, and then plated at 1×10\hat{ }4 cells per well for incubation at 37° C. for 30 min. Anti-hCLDN18.2 chimeric antibodies and the control antibody IMAB362 were diluted by using RPMI1640 with 10% FBS, and then added into the target cell plate at a final concentration from 100 to 0.0017 μg/ml. Log-phase Jurkat-NFAT-luc-FcγRIIIA-V176 cells were also added into the above plate at 1×10\hat{ }4 cells per well. Plates were incubated at 37° C. for 6 hours. Next, the cell culture plates were allowed to equilibrate to room temperature for 30 minutes. The Cell Titer-Glo Luminescent Cell Viability Assay Kit was used for cell viability analysis at a room temperature by using the microplate reader (Thermo VARIOSKAN FLASH 3001).

Based on reporter readout curve, EC50 can be calculated and used for evaluation of ADCC effect. As shown in FIG. 3B using MKN45-CLDN18.2-high cell, though IMAB362 had few points to calculate EC50, the two curves suggested that 18B10-C had a better ADCC activity than IMAB362. As shown in FIG. 3D in MKN45-CLDN18.2-medium cell, 18B10-C had over 50-fold increase in ADCC potency as measured by EC50 than IMAB362. These results suggested that cell with medium expression of CLDN18.2 may differentiate the 18B10-C antibody from IMAB362 better than high expression cell. No CDC activity was seen on MKN45-hCLDN18.2 cell (data not shown).

3. Binding and Cytotoxic Effect on NUGC4 Cell

NUGC4 represents a gastric cell line with a similar expression level of hCLDN18.2 to those from gastric cancer patients.

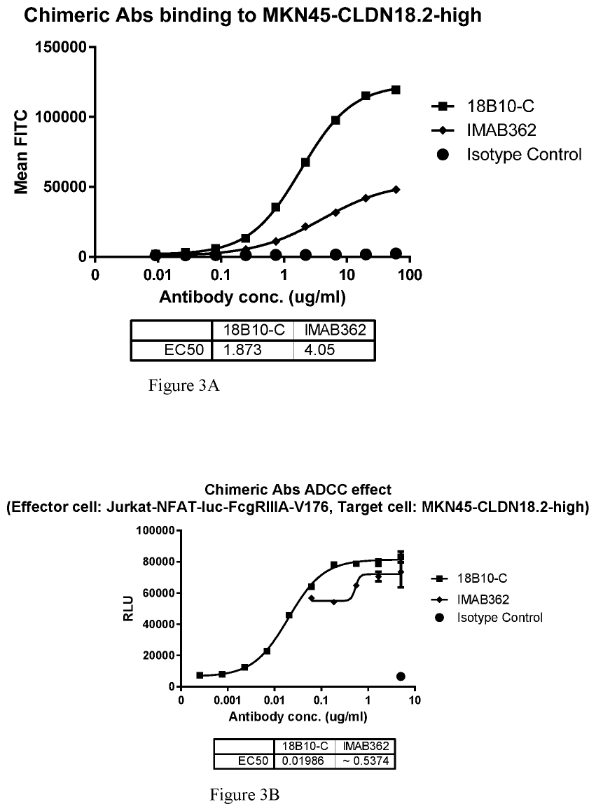

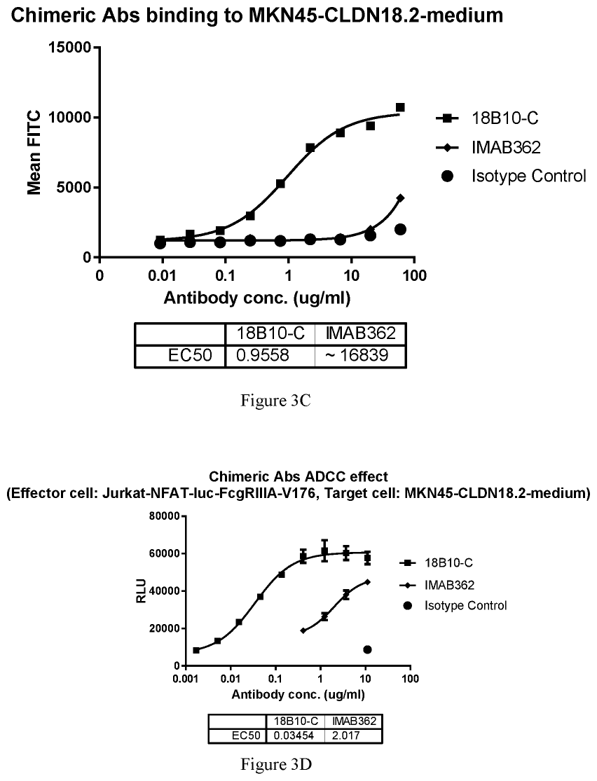

Cell binding assay and ADCC reporter assay were performed following the same method above (see section 2 of this example). As shown in FIGS. 4A and 4C, 5 of the 6 chimeric antibodies bound to NUGC4 cell with EC50 around 10 μg/ml (see Table 5), except for 26G6-C and 59A9-C. 26G6-C bound to NUGC4 with a higher EC50 (67 μg/ml), indicating a lower affinity. 59A9-C showed both a higher EC50 (19 g/ml) and a lower maximum signal. In addition, FIGS. 4B and 4D showed a similar trend of their ADCC activity against the NUGC4 cells. Due to two separately experiments, the EC50 and maximum signals may vary between FIG. 4B and FIG. 4D. Importantly, all the tested chimeric antibodies demonstrated a better ADCC activity than IMAB362, especially 18B10-C with an over 40-fold increase than IMAB362. No CDC activity was seen on NUGC4 cell (data not shown).

Table 5 summarized FACS binding data of all chimeric antibodies and IMAB362 to HEK293-CLDN18.2 and NUGC4 cells.

TABLE 5

| FACS binding | | |
|---|---|---|
| (EC50, ug/ml) | HEK293-CLDN18.2 | NUGC4 |
| 7C12-C | 0.63 | 12.97 |
| 11F12-C | 0.61 | 10.02 |
| 12E9-C | 0.65 | 9.37 |
| 26G6-C | 1.10 | 67.11 |
| IMAB362 | 0.42 | >100 |
| 59A9-C | 1.35 | 19.08 |
| 18B10-C | 0.96 | 7.80 |

4. Specificity of Chimeric CLDN18.2 Antibodies

CLDN18.2 has only several amino acids different from CLDN18.1 that exists in many normal tissues and organs. The antibody binding specificity to CLDN18.2 is very important. Cell binding assay was same as above (see section 1 of this example). FIG. 5 showed binding of 18B10-C and IMAB362 to CLDN18.2—or CLDN18.1-expressing HEK293 cell. Both antibodies only bound to the CLDN18.2—but not CLDN18.1-expressing cell. Other chimeric antibodies also had a similar good selectivity (data not shown).

### Example 8: Epitope Binning

Hybridoma Antibodies Compete the Binding of Benchmark Antibodies to CLDN18.2-Expressing Cells

Log-phase MKN45-CLDN18.2=high cells were resuspended in FACS washing buffer (PBS with 2% BSA), and then added into 96-well V bottom plate at density of 1×10\hat{ }5 cells per well. The diluted hybridoma antibodies or IMAB362-mIgG2a (final concentration: from 100 to 0.01 μg/ml) were added into the plate. The plate was incubated at 4° C. for 1 hour to allow antibody fully occupation of antigen on cell surface. Cells were washed 2 times with FACS washing buffer, and 10 g/ml IMAB362 or 5 μg/ml 18B10-C were added to cells for further incubation at 4° C. for 1 hour. Then cells were washed 3 times and incubated with Goat anti-hIgG (H+L)-FITC (1:200 dilution). Finally, cells were washed 3 times by FACS washing buffer and analyzed by Flow Cytometry.

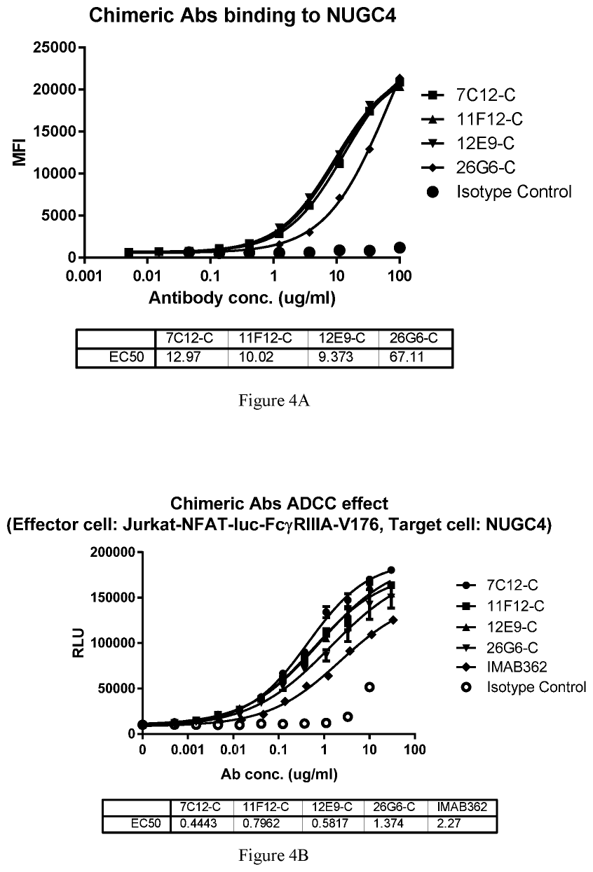

As shown in FIGS. 6A and 6B, hybridoma antibody 18B10 could completely block the binding of IMAB362 to MKN45-CLDN18.2 cells, indicating 18B10 may have higher binding affinity than IMAB362 but relying on similar or nearby amino acids (Table 6).

TABLE 6
Be competed one

| Competitors | IMAB362 | 7C12-C | 11F12-C | 12E9-C | 26G6-C | 18B10-C |
|---|---|---|---|---|---|---|
| IMAB362-mIgG2a | partial | partial | partial | partial | partial | partial |
| 7C12 | ✓ | partial | partial | partial | partial | — |
| 11F12 | ✓ | partial | partial | partial | partial | — |
| 12E9 | ✓ | partial | partial | partial | partial | — |
| 26G6 | ✓ | partial | partial | partial | partial | — |
| 18B10 | ✓ | — | — | — | — | partial |

✓: complete blockade;
partial: partial blockade;
—: undetected

### Example 9: Epitope Mapping of Selected Antibodies Via Site-Directed Mutagenesis on the CLDN18.2 Amino Acids Different with CLDN18.1

1. Generation of Human CLDN18.2-mRFP and Human CLDN18.1-mRFP Constructs

The cDNA coding for human CLDN18.1 (amino acid 1-261, SEQ ID NO: 31)-mRFP1 (amino acid 1-225) and human CLDN18.2 (amino acid 1-261, SEQ ID NO: 30)-mRFP1 (amino acid 1-225) were synthesized in vitro (SEQ ID NO: 52 and SEQ ID NO: 53 are the amino acid sequences, respectively). The PCR product was then cloned into the pcDNA3.1 (+) vector by method of homologous recombination using Syno assembly mix reagent (Synbio) following manufacturer's instructions. Plasmid was purified by using QIAGEN Plasmid Mega Kit (QIAGEN).

According to the sequence of human CLDN18.1 and CLDN18.2 (Genbank accession number: splice variant 1 (CLDN18.1): NP_057453, NM_016369, and splice variant 2 (CLDN18.2): NM_001002026, NP_001002026), 8 different amino acids are located between 28-70, which may be the determinant of the specific binding to human CLDN18.2 not to CLDN18.1. Using wild-type human CLDN18.2-mRFP plasmid generated above as template, two segments of an integrated sequence were generated with the primers. The variants of human CLDN18.2-mRFP with single amino acid changed into that of human CLDN18.1 at the designated position were amplified by overlapping PCR using the primers. The specific mutations are on Q29M, N37D, A42S, N45Q, Q47E, E56Q, G65P and L69I. Variants of human CLDN18.1-mRFP with single amino acid changed at designated position were amplified by overlapping PCR using primers. The specific mutations are on M29Q, D37N, S42A, Q45N, E47Q, Q56E, P65G, I69L. The PCR product was then cloned into the pcDNA3.1 (+) vector by method of homologous recombination. The human CLDN18.2-mRFP variants were identified and confirmed by sequencing the individual positive clones.

Subsequently, these plasmids of mutants and wild-type human CLDN18.2-mRFP or human CLDN18.1-mRFP were transfected into HEK293 cell line. First, 5×10⁶ HEK293 cells were seeded into 60 mm dish at a ratio of 60%˜80% for transfection. 10 μg DNA in 400 μl 1×HBS and 10 μl 25 kDa linear PEI transfection reagent (dissolved in 1×HBS, 1 mg/ml stock solution) was mixed to reach a DNA/PEI ratio of 1:2.5. Next the mixture was added into HEK293 cell culture drop by drop. After 6-8 hours, the transfected cells were replaced with complete DMEM for overnight. At 24 hours after transfection, cells were collected for FACS analysis using chimeric antibodies.

Amino acid sequence of human CLDN18.1
(SEQ ID NO: 31)
MSTTTCQVVAFLLSILGLAGCIAATGMDMWSTQDLYDNPVTSVFQYEGL
WRSCVRQSSGFTECRPYFTILGLPAMLQAVRALMIVGIVLGAIGLLVSI
FALKCIRIGSMEDSAKANMTLTSGIMFIVSGLCAIAGVSVFANMLVTNF
WMSTANMYTGMGGMVQTVQTRYTFGAALFVGWVAGGLTLIGGVMMCIAC
RGLAPEETNYKAVSYHASGHSVAYKPGGFKASTGFGSNTKNKKIYDGGA
RTEDEVQSYPSKHDYV
Amino acid sequence of human CLDN18.1-mRFP1
(SEQ ID NO: 52)
MSTTTCQVVAFLLSILGLAGCIAATGMDMWSTQDLYDNPVTSVFQYEGL
WRSCVRQSSGFTECRPYFTILGLPAMLQAVRALMIVGIVLGAIGLLVSI
FALKCIRIGSMEDSAKANMTLTSGIMFIVSGLCAIAGVSVFANMLVTNF
WMSTANMYTGMGGMVQTVQTRYTFGAALFVGWVAGGLTLIGGVMMCIAC
RGLAPEETNYKAVSYHASGHSVAYKPGGFKASTGFGSNTKNKKIYDGGA
RTEDEVQSYPSKHDYVMASSEDVIKEFMRFKVRMEGSVNGHEFEIEGEG
EGRPYEGTQTAKLKVTKGGPLPFAWDILSPQFQYGSKAYVKHPADIPDY
LKLSFPEGFKWERVMNFEDGGVVTVTQDSSLQDGEFIYKVKLRGTNFPS
DGPVMQKKTMGWEASTERMYPEDGALKGEIKMRLKLKDGGHYDAEVKTT
YMAKKPVQLPGAYKTDIKLDITSHNEDYTIVEQYERAEGRHSTGA
Amino acid sequence of human CLDN18.2
(SEQ ID NO: 30)
MAVTACQGLGFVVSLIGIAGIIAATCMDQWSTQDLYNNPVTAVFNYQGL
WRSCVRESSGFTECRGYFTLLGLPAMLQAVRALMIVGIVLGAIGLLVSI
FALKCIRIGSMEDSAKANMTLTSGIMFIVSGLCAIAGVSVFANMLVTNF
WMSTANMYTGMGGMVQTVQTRYTFGAALFVGWVAGGLTLIGGVMMCIAC
RGLAPEETNYKAVSYHASGHSVAYKPGGFKASTGFGSNTKNKKIYDGGA
RTEDEVQSYPSKHDYV
Amino acid sequence of human CLDN18.2-mRFP1
(SEQ ID NO: 53)
MAVTACQGLGFVVSLIGIAGIIAATCMDQWSTQDLYNNPVTAVFNYQGL
WRSCVRESSGFTECRGYFTLLGLPAMLQAVRALMIVGIVLGAIGLLVSI
FALKCIRIGSMEDSAKANMTLTSGIMFIVSGLCAIAGVSVFANMLVTNF
WMSTANMYTGMGGMVQTVQTRYTFGAALFVGWVAGGLTLIGGVMMCIAC
RGLAPEETNYKAVSYHASGHSVAYKPGGFKASTGFGSNTKNKKIYDGGA
RTEDEVQSYPSKHDYVMASSEDVIKEFMRFKVRMEGSVNGHEFEIEGEG
EGRPYEGTQTAKLKVTKGGPLPFAWDILSPQFQYGSKAYVKHPADIPDY
LKLSFPEGFKWERVMNFEDGGVVTVTQDSSLQDGEFIYKVKLRGTNFPS
DGPVMQKKTMGWEASTERMYPEDGALKGEIKMRLKLKDGGHYDAEVKTT
YMAKKPVQLPGAYKTDIKLDITSHNEDYTIVEQYERAEGRHSTGA

2. Binding of CLDN18.2 Chimeric Antibodies to Site-Mutated HEK293-CLDN18.2 or HEK293-CLDN18.1 Cell

The transfected HEK293-CLDN18.2 or HEK293-CLDN18.1 cell were resuspended in PBS with 2% BSA at density of 10\hat{ }5/well, 100 μl/well. Cells were washed 3 times by FACS washing buffer (PBS+2% FBS) and incubated with 100 μl/well 10 μg/ml chimeric antibodies and IMAB362 each well at 4° C. for 30 min. Next, cells were washed 3 times by FACS washing buffer and incubated with 100 μl/well goat anti-hIgG (H+L)-FITC (1:200 dilution) at 4° C. for another 30 min. Finally, cells were washed 3 times by FACS washing buffer and analyzed by Flow Cytometry. To analyze binding to CLDN18.2 transfected cells, the RFP positive cells were used for control gating.

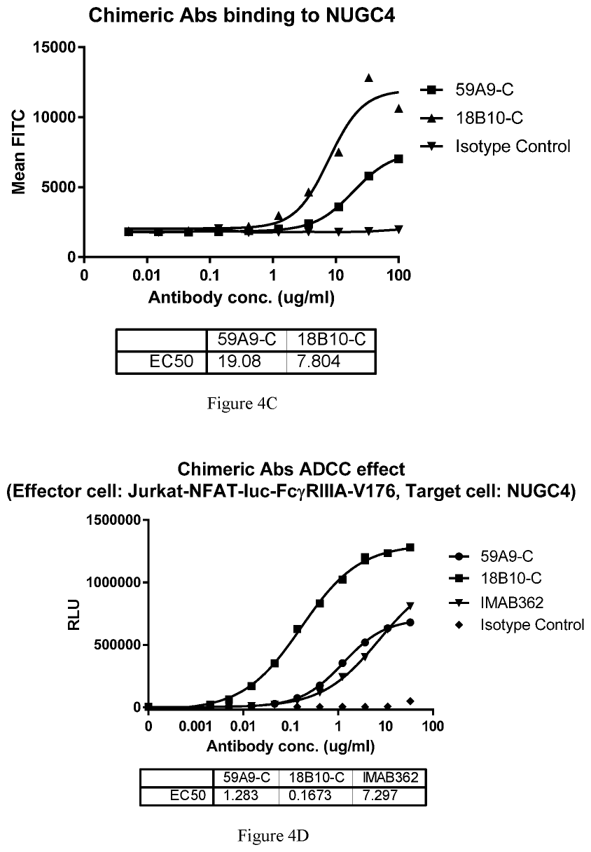

The percentage of binding signal of these chimeric antibodies to mutated CLDN18.2 variants relative to that the wild-type was calculated and summarized in Table 7. As shown in FIG. 7A-71, binding of 18B10-C was completely lost when E56 was mutated to Q. This change also applied to IMAB362 and other chimeric antibodies, except for 59A9-C. In addition, we found that other amino acids, such as A42, N45, also contributed to binding of IMAB362 and other antibodies at some extent but not so for 18B10-C.

TABLE 7
The binding percentage of mutated human CLDN18.2
as compared with wild-type CLDN18.2 (%)

| Mutations on | | | | | | | |
|---|---|---|---|---|---|---|---|
| CLDN18.2 | IMAB362 | 7C12-C | 11F12-C | 12E9-C | 26G6-C | 59A9-C | 18B10-C |
| Wild-type | 100.00 | 100.00 | 100.00 | 100.00 | 100.00 | 100.00 | 100.00 |
| Q29M | 113.89 | 83.52 | 107.78 | 106.33 | 106.53 | 90.37 | 83.50 |
| N37D | 121.11 | 80.20 | 102.19 | 102.71 | 117.72 | 92.66 | 105.63 |
| A42S | 15.76 | 38.69 | 56.86 | 55.10 | 82.23 | 90.02 | 69.23 |
| N45Q | 18.87 | 52.10 | 57.38 | 50.77 | 88.69 | 65.04 | 91.97 |
| Q47E | 108.17 | 74.74 | 86.13 | 89.07 | 116.12 | 87.47 | 109.00 |
| E56Q | 0.83 | 0.48 | 0.27 | 0.35 | 0.34 | 52.74 | 6.57 |
| G65P | 119.26 | 79.44 | 92.67 | 96.73 | 118.87 | 85.55 | 118.11 |
| L69I | 89.27 | 90.38 | 74.28 | 58.10 | 79.74 | 105.74 | 111.70 |

### Example 10: Generation and Characterization of Humanized Antibodies

1. Generation, Expression and Purification of Humanized Antibodies

18B10

Human germline framework sequence VK/4-1 for light chain and VH/1-46 for heavy chain were used for CDR grafting, respectively.

Heavy chain (HC) variants 1, 2 and 3 were obtained by direct grafting the three CDRs to the germline sequence (18B10 HC germline, SEQ ID NO: 23) and back mutation of R71I, T73K for HC variant 1 (Hu18B10_Ha, SEQ ID NO: 25), back mutation of R71I, T73K, T28S, M69L for HC variant 2 (Hu18B10_Hb, SEQ ID NO: 27) and back mutation of R71I, T73K, T28S, M69L, R38K, M48I for HC variant 3 (Hu18B10_Hc, SEQ ID NO: 29), respectively.

(1) Germline Sequence for 18B10 HC:

VH/1-46(18B10-germline, SEQ ID NO: 23):
QVQLVQSGAEVKKPGASVKVSCKASGYTFTSYYMHWVRQAPGQGLEWMG
IINPSGGSTSYAQKFQGRVTMTRDTSTSTVYMELSSLRSEDTAVYYCAR
VH/1-46 variant 1 (Hu18B10 Ha, SEQ ID NO: 25):
QVQLVQSGAEVKKPGASVKVSCKASGYTFTGYNMNWVRQAPGQGLEWMG
NIDPYYGGTSYNQKFKGRVTMTIDKSTSTVYMELSSLRSEDTAVYYCAR
MYHGNAFDYWGQGTTVTVSS
VH/1-46 variant 2 (Hu18B10 Hb, SEQ ID NO: 27):
QVQLVQSGAEVKKPGASVKVSCKASGYSFTGYNMNWVRQAPGQGLEWMG
NIDPYYGGTSYNQKFKGRVTLTIDKSTSTVYMELSSLRSEDTAVYYCAR
MYHGNAFDYWGQGTTVTVSS
VH/1-46 variant 3 (Hu18B10 Hc, SEQ ID NO: 29):
QVQLVQSGAEVKKPGASVKVSCKASGYSFTGYNIMNWVKQAPGQGLEWI
GNIDPYYGGTSYNQKFKGRVTLTIDKSTSTVYMELSSLRSEDTAVYYCA
RMYHGNAFDYWGQGTTVTVSS

Light chain (LC) variant 1 and 2 were obtained by direct grafting the three CDRs to germline sequence (18B10 LC germline, SEQ ID NO: 24) and no back mutation for variant 1 (Hu18B10_La, SEQ ID NO: 26) and S63T, I21M for LC variant 2 (Hu18B10_Lb, SEQ ID NO: 28), respectively.

(2) Germline Sequence for 18B10 LC:

VK/4-1 (18B10 LC germline, SEQ ID NO: 24)
DIVMTQSPDSLAVSLGERATINCKSSQNNKNYLAWYQQKPGQPPKLLIY
WASTRESGVPDRFSGSGSGTDFTLTISSLQAEDVAVYYCQQYYSTP
VK/4-1 variant 1 (Hu18B10_La, SEQ ID NO: 26)
DIVMTQSPDSLAVSLGERATINCKSSQSLLNSGNLKNYLTWYQQKPGQP
PKLLIYWASTRKSGVPDRFSGSGSGTDFTLTISSLQAEDVAVYYCQNDY
SYPLTFGGGTKVEIK
VK/4-1 variant 2 (Hu18B10_Lb, SEQ ID NO: 28)
DIVMTQSPDSLAVSLGERATMNCKSSQSLLNSGNLKNYLTWYQQKPGQP
PKLLIYWASTRKSGVPDRFTGSGSGTDFTLTISSLQAEDVAVYYCQNDY
SYPLTFGGGTKVEIK

The combination of the above heavy chain variable regions and light chain variable regions generate the following humanized 18B10 antibodies: 18B10-HaLa (having a VH of SEQ ID NO: 25 and a VL of SEQ ID NO: 26), 18B10-HbLa (having a VH of SEQ ID NO: 27 and a VL of SEQ ID NO: 26), 18B10-HcLa (having a VH of SEQ ID NO: 29 and a VL of SEQ ID NO: 26), 18B10-HaLb (having a VH of SEQ ID NO: 25 and a VL of SEQ ID NO: 28), 18B10-HbLb (having a VH of SEQ ID NO: 27 and a VL of SEQ ID NO: 28), 18B10-HcLb (having a VH of SEQ ID NO: 29 and a VL of SEQ ID NO: 28).

The humanized variants of the heavy chain and light chain of 18B10 are linked to human IgG1 heavy chain constant region and kappa light chain constant region as shown below:

Human IgG1 heavy chain constant region
(SEQ ID NO: 49):
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
Human Kappa light chain constant region
(SEQ ID NO: 50):
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

The variable regions of the above heavy chain and light chain cDNAs were synthesized and fused with the constant region of human IgG1 and human kappa. The heavy chain and light chain of the selected antibody genes were cloned into an expression vector and the large-scale DNA was prepared using Plasmid Maxiprep System from Qiagen. Transfection was carried out using the ExpiFectamine™ CHO Reagent from Invitrogen according to the manufacturer's protocol. Supernatants were harvested when the cell viability was around 60%. The cell culture supernatant was filtered through 0.22 um filtration capsule to remove the cell debris. Load the supernatant onto a pre-equilibrated Protein-A affinity column. Then Protein A resin inside the column was washed with equilibration buffer (PBS), and 25 mM citrate (pH3.5) was used to elute the antibody. The pH was adjusted to about 6.0-7.0 with 1 M Tris-base (pH 9.0). The endotoxin was controlled below 1 EU/mg. The purified antibody was then characterized by SDS-PAGE and SEC-HPLC.

Binding to Human and Mouse CLDN18.2

Binding of the humanized antibodies were tested following the same method as described in Example 5.

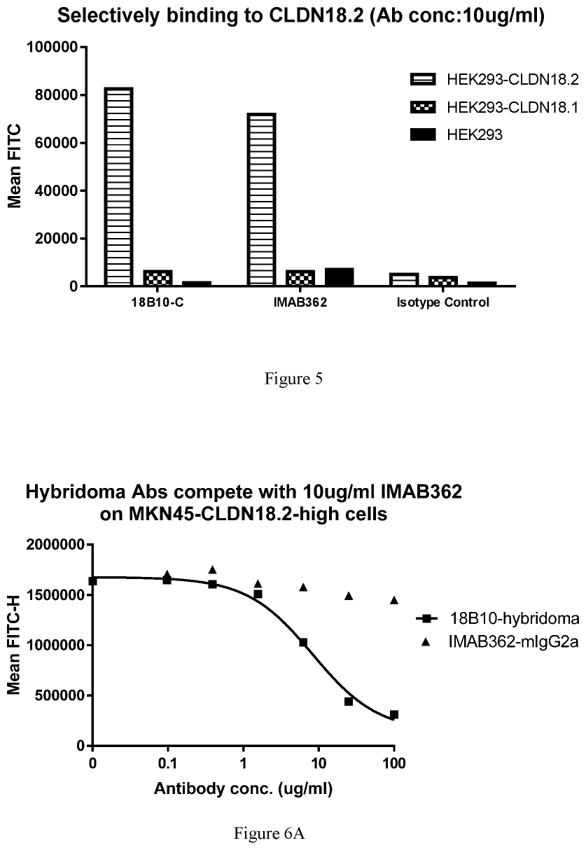

As shown in FIG. 8A, all humanized variants were tested head to head with the chimeric one in order to screen the best. All the variants retained their binding completely. Next, 18B10-HaLa, which had only one back mutation, was tested for its binding to HEK293-mouse CLDN18.2 cells (FIG. 8B). 18B10-HaLa could bind well to mouse CLDN18.2 with a better potency and a higher MFI than IMAB362, indicating that 18B10-HaLa had a good cross-reactivity to mouse.

3. Affinity Analysis of Humanized CLDN18.2 Antibodies by KinExA

18B10-HaLa and IMAB362 were evaluated head to head by KinExA for their affinity binding to CLDN18.2 expressing cells. Following KinExA 4000 (Sapidyne Instruments Inc.)'s instruction, 200 mg PMMA hard beads (Sapidyne, #440176) were coated with 30 μg Goat anti-human IgG Fe antibody for 2 h, and then blocked by 10 mg/ml BSA for 1 h. Two gastric cell lines, NUGC4 and KATOIII (ATCC, Cat #HTB-103), were collected at log-phase and mixed with 0.2 nM 18B10-HaLa or IMAB362. The cell-antibody mixture was 2-fold diluted using 0.2 nM 18B10-HaLa or IMAB362 and incubated at room temperature for 3 h. Amount of the free antibodies was increasing along with dilution. These free antibodies were captured by Goat anti-human IgG Fc coated beads, and subsequently labeled by 1 μg/ml Alexa Fluor 647-anti-human IgG for readout.

The binding affinity of each antibody was summarized in Table 8. Kd of 18B10-HaLa binding to NUGC4 cell and KATOIII was approximately 0.3 nM, which was over 8-fold higher than that of IMAB362. This was consistent with the above FACS binding results.

TABLE 8
Kd of CLDN18.2 antibodies to gastric cell lines

| Kd (nM) | 18B10-HaLa | IMAB362 |
|---|---|---|
| NUGC4 | 0.303 | 2.58 |
| KATOIII | 0.315 | ND |

4. CDC Assay on HEK293-CLDN18.2 Cell

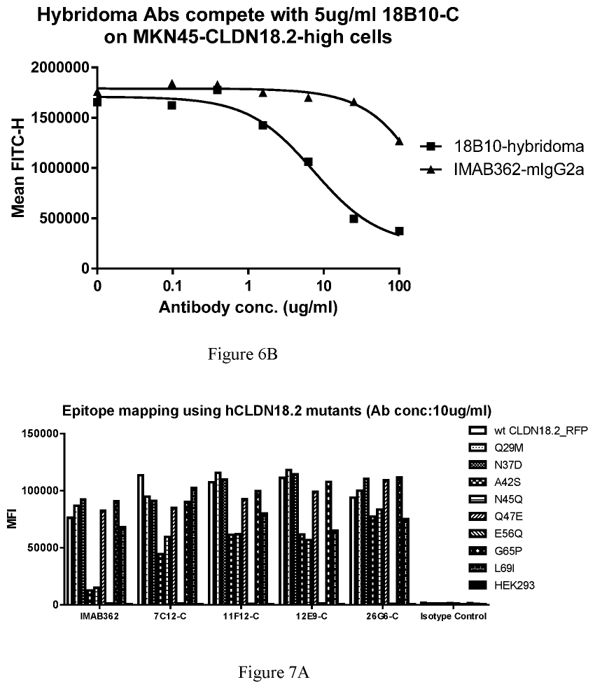

Similar to method above (see section 1 of Example 7), 18B10-HaLa was tested head to head with IMAB362 in the CDC activity assay. As shown in FIG. 9, 18B10-HaLa had more than 20-fold higher CDC activity than IMAB362. The percentage of 18B10-HaLa-dependent specific cell killing reached to 86% at a concentration of 0.3 μg/ml, while IMAB362 had no cell killing at the same concentration.

5. Binding and Cytotoxic Effect on MKN45-CLDN18.2 Cell

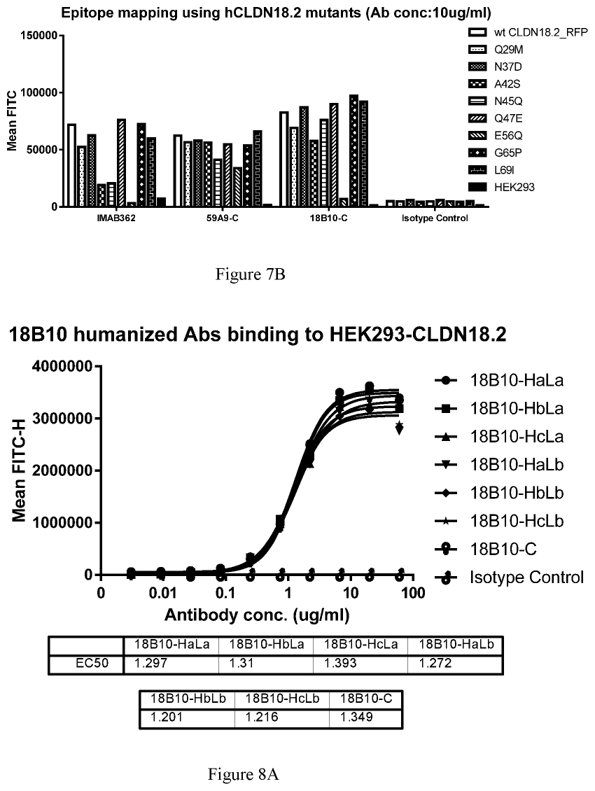

Cell binding assay was same as above. As shown in FIG. 10A, all humanized variants of 18B10 bound to cells with a comparable affinity with chimeric 18B10. 18B10-HaLa, with only one back mutation, was selected for further ADCC activity study.

ADCC activity was tested using Jurkat-NFAT-luc-FcγRIIIA-V176 cells as effector cells and MKN45-CLDN18.2 cells as target cells. Assay protocol was same as above (see section 2 of Example 7). As shown in FIG. 10B, 18B10-HaLa had a much lower EC50 (0.05 μg/ml) than IMAB362, consistent with that of chimeric 18B10.

6. Binding and Cytotoxic Effect on NUGC4 Cell

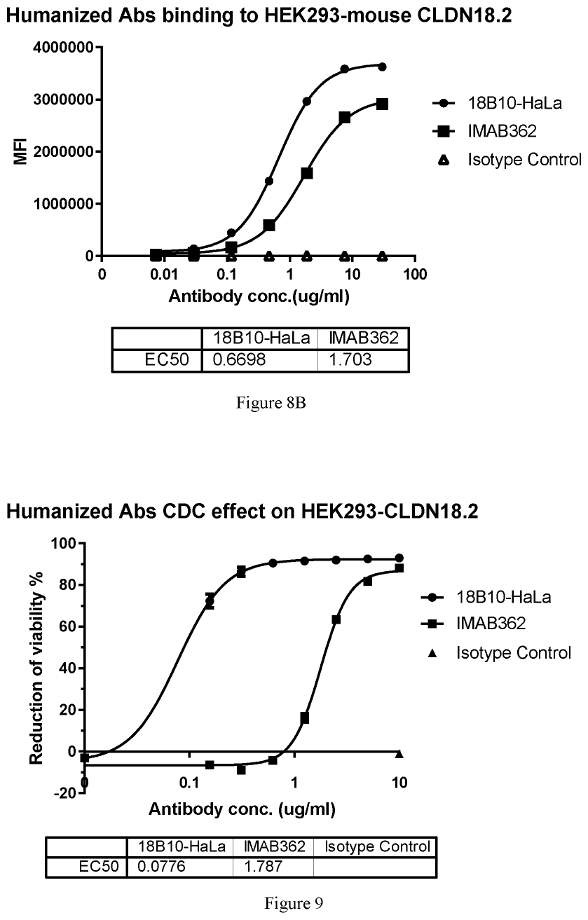

Cell binding and ADCC assays were same as above. FIG. 11A showed the results of binding affinity of 18B10-HaLa to NUGC4 cells. FIG. 11B showed a much better ADCC potency of 18B10-HaLa (EC50-0.59 μg/ml) as compared to IMAB362.

7. ADCC Assay Using NUGC4 as Target Cell and PBMC as Effector Cell

Log-phase NUGC4 cells were resuspended in RPMI1640 with 10% FBS. Cells were pre-seeded into 96-well U bottom plate at 1×10\hat{ }4 cells per well. Anti-CLDN18.2 antibodies and IMAB362 were gradient diluted in RPMI1640 with 10% FBS and added into the plate above at a final concentration from 200 to 0.2 μg/ml and incubated at 37° C. for 30 min. Frozen PBMC from Miao Shun (Shanghai) Biological & Technology Co., Ltd were removed from liquid nitrogen and put into 37° C. water bath immediately. After centrifugation, cells were resuspended in RPMI1640 plus 10% FBS and the seeded into 96-well U bottom plate mentioned above at 40×10∝cells per well. The plate was then placed in the incubator at 37° C. for 5 hours.

After incubation, the plate was equilibrated to 22° C. LDH was detected by using Promega CytoTox-ONE Homogeneous Membrane Integrity Assay Kit (G7892) or the CytoTox 96® Non-Radioactive Cytotoxicity Assay (G1780). After adding the Lysis, Reagent and Stop Solutions following manufacturer's instruction, fluorescence was measured under an excitation wavelength of 560 nm and an emission wavelength of 590 nm (G7892), or the absorbance at 490 nm or 492 nm (G1780).

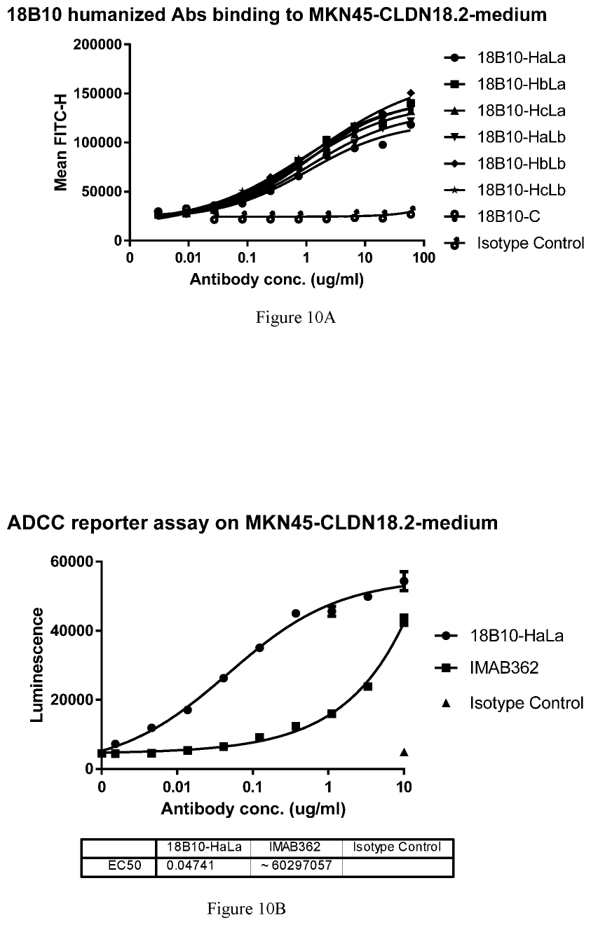

FIG. 12 showed a representative data using PBMC as effector cell. 18B10-HaLa showed a much better ADCC potency than IMAB362. Due to non-fit to regression curve, EC50 may not be calculated accurately.

8. Epitope Mapping of the Selected Antibodies Using Site-Directed Mutagenesis on Human CLDN18.2

Using the same method and human CLDN18.2-mRFP plasmid as Example 9, 42 amino acids between human CLDN18.2 28-80 as listed below were replaced by alanine one at a time. These variants were amplified by overlapping PCR using primers. The specific mutations are Q28A, Q29A, W30A, S31A, T32A, Q33A, D34A, L35A, Y36A, N37A, N38A, V40A, T41A, V43A, F44A, N45A, Y46A, Q47A, L49A, W50A, R51A, S52A, V54A, R55A, E56A, E56A, S57A, S58A, F60A, T61A, E62A, R64A, Y66A, F67A, T68A, L69A, L70A, L72A, M75A, L76A, Q77A, V79A, R80A. The PCR product was then cloned into the pcDNA3.1 (+) vector by method of homologous recombination using Syno assembly mix reagent (Synbio) following manufacturer's instructions. Plasmid was purified by using QIAGEN Plasmid Mega Kit (QIAGEN).

Subsequently, these plasmids of mutants and wild-type CLDN18.2-mRFP were transfected into HEK293 cell. As Example 9, cells were analyzed by flow cytometry 24 hours after transfection.

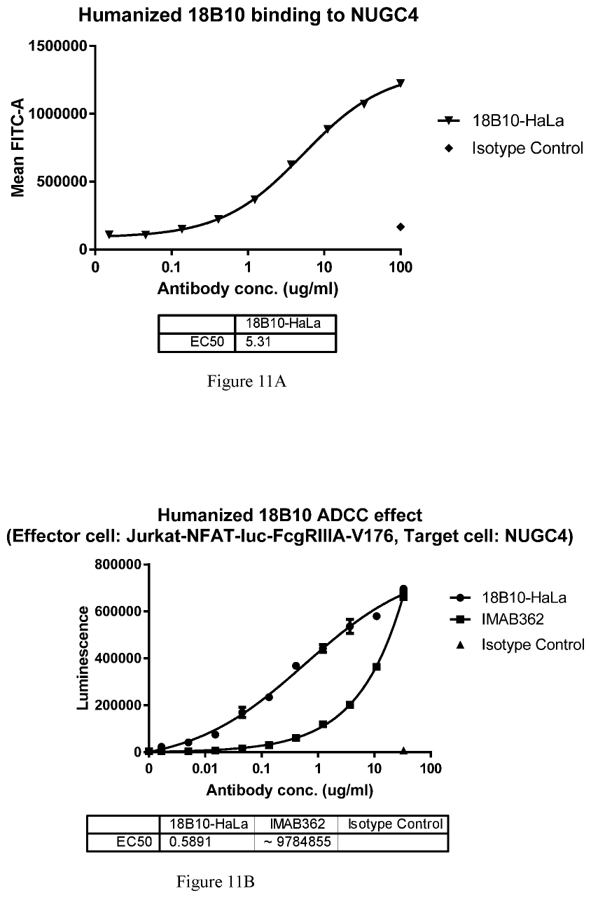

As shown in FIG. 13A, binding of 18B10-HaLa was completely lost (binding percentage<10%) when W30, L49, W50, E56 were mutated to A, indicating these 4 amino acids are critical for its binding to human CLDN18.2. Especially E56 is the most important one to constitute the binding epitope. Besides these 4 critical ones, several other amino acids also effect the binding (binding percentage between 10% and 25%) once they are replaced by alanine, such as R51, F60, E62, R80. FIG. 13B showed the binding of 59A9-C to the site-mutated CLDN18.2, which was only partially dependent on E56 (binding percentage about 22%). The binding percentage of the mutated CLDN18.2 as compared with the wild-type to the antibodies was summarized in Table 9.

TABLE 9
The binding percentage of mutated CLDN18.2 as compared
with wild-type CLDN18.2 (%)

| Mutations on CLDN18.2 | 18B10-HaLa | 59A9-C |
|---|---|---|
| Wild-type | 100.00 | 100.00 |
| Q28A | 28.47 | 46.38 |
| Q29A | 82.85 | 54.83 |
| W30A | 0.38 | 3.20 |
| S31A | 92.24 | 76.85 |
| T32A | 91.56 | 85.59 |
| Q33A | 57.11 | 59.78 |
| D34A | 97.47 | 96.29 |
| L35A | 89.80 | 75.44 |
| Y36A | 79.92 | 78.03 |
| N37A | 61.20 | 60.72 |
| N38A | 66.62 | 57.63 |
| V40A | 103.48 | 128.07 |
| T41A | 60.27 | 41.63 |
| V43A | 36.83 | 59.41 |
| F44A | 59.86 | 70.03 |
| N45A | 36.19 | 35.16 |
| Y46A | 51.55 | 14.98 |
| Q47A | 105.11 | 57.45 |
| L49A | 1.20 | 2.32 |
| W50A | 0.86 | 5.05 |
| R51A | 39.39 | 22.12 |
| S52A | 86.58 | 98.97 |
| V54A | 84.22 | 87.24 |
| R55A | 49.94 | 49.69 |
| E56A | 0.27 | 21.92 |
| S57A | 112.14 | 77.16 |
| S58A | 62.08 | 39.38 |
| F60A | 26.32 | 64.43 |
| T61A | 84.08 | 51.64 |
| E62A | 8.42 | 23.13 |
| R64A | 87.70 | 51.00 |
| Y66A | 53.19 | 52.45 |
| F67A | 76.37 | 79.89 |
| T68A | 70.92 | 71.30 |
| L69A | 88.60 | 54.83 |
| L70A | 75.64 | 82.08 |
| L72A | 56.61 | 53.03 |
| M75A | 75.98 | 73.58 |
| L76A | 43.12 | 52.06 |
| Q77A | 80.06 | 63.42 |
| V79A | 43.72 | 39.11 |
| R80A | 27.27 | 13.64 |

### Example 11: Antibody Drug Conjugate (ADC) Internalization and Cytotoxicity

18B10-HaLa and control hIgG1 were conjugated with vcMMAE using the MC-vc-PAB-MMAE KIT (Levena Biopharma, Cat #SET0201). The Drug to Antibody Ratio (DAR) of chimeric 18B10-HaLa was 4.05, while that of IMAB362 and control hIgG1 was 2.9 and 4.96, respectively. The effect of 18B10-HaLa-vcMMAE on cell viability was evaluated by using a colorimetric assay that detects cellular metabolic activities.

Log-phase HEK293-CLDN18.2, NUGC4 or MKN45-CLDN18.2-high cells were resuspended in their corresponding culture medium, and then added into cell culture plate at 1×10∝cells per well, 50 μl/well for incubation at 37° C. overnight. Next Ab-vcMMAE, control hIgG1-vcMMAE and Ab were gradient diluted and added into each well, 50 μl/well. A final concentration of 4.75 nM of vcMMAE was used as a positive control for cytotoxicity. 72 hours later, 100 μl/well of detection reagent from the CellTiter-Glo Luminescent Cell Viability Assay Kit was added to each well for 10 minutes at room temperature, before readout using the microplate reader.

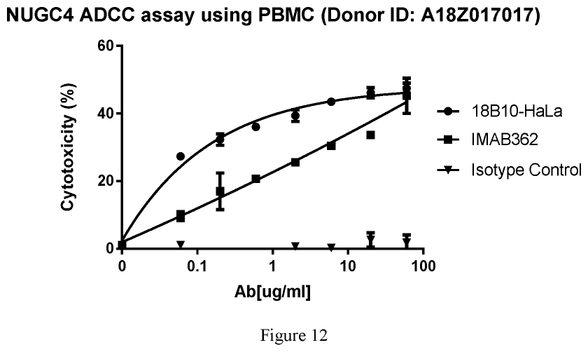

As shown in FIG. 14A, both 18B10-HaLa-vcMMAE and IMAB362-vcMMAE but not the control hIgG1-vcMMAE induced cytotoxicity on HEK293-CLDN18.2 cell, indicating the cytotoxicity was hCLDN18.2-specific. While 18B10-HaLa and IMAB362 alone had no cytotoxicity against the target cells (data not shown), indicating the observed cytoxicity was vcMMAE mediated. FIG. 14B showed the cytotoxic effect on NUGC4, a gastric cancer cell. 18B10-HaLa-vcMMAE demonstrated a dose-dependent cell growth inhibition starting at a concentration of 0.03 μg/ml. In contrast, IMAB362-vcMMAE only inhibited cell growth at 10 μg/ml, a much higher concentration. In another gastric cancer cell MKN-45 transfected with CLDN18.2 (high expression), 18B10-HaLa-vcMMAE reached a maximum cell killing of 86%, which was also higher than IMAB362 (60%) (as shown in FIG. 14C).

It has been well studied that ADC functions via antigen binding and internalization into target cells. The drug conjugated with antibody could not be released and kill cells until being internalized and transferred to lysosome for degradation. We used this assay as preliminary estimation of internalization feature of 18B10-HaLa. The results suggest that it has a potential internalization activity and can be developed as ADC therapeutic drug.

### Example 12: In Vivo Efficacy Evaluation of Humanized CLDN18.2 Antibodies in MKN45-CLDN18.2-High Xenograft Model

1. Anti-Tumor Efficacy on MKN45-CLDN18.2-High Xenograft Model Using Nude Mice

In vitro study (Example 10) showed humanized CLDN18.2 antibodies could induce ADCC effect on MKN45-CLDN18.2-high cells (Example 1). Therefore, in vivo model was established and used for evaluation of anti-tumor activity. Briefly, each female Balb/c nude mice was inoculated with 5×10\hat{ }6 MKN45-CLDN18.2-high cells with 50% matri-gel (BD) by s.c. injection on the right flank. 12 days after inoculation, 24 mice with tumor size around 70 mm\hat{ }3 were selected and randomized into 3 μgroups (n=8). Then the mice were treated with isotype control or humanized CLDN18.2 antibodies at a dose of 0.3 mg/kg, twice a week for 3 weeks by i.p. injection. Animals were sacrificed at the end of the study with C02 inhalation. Tumor size and volume were measured 2-3 times a week. Results were analyzed using Prism GraphPad and expressed as mean±S.E.M.

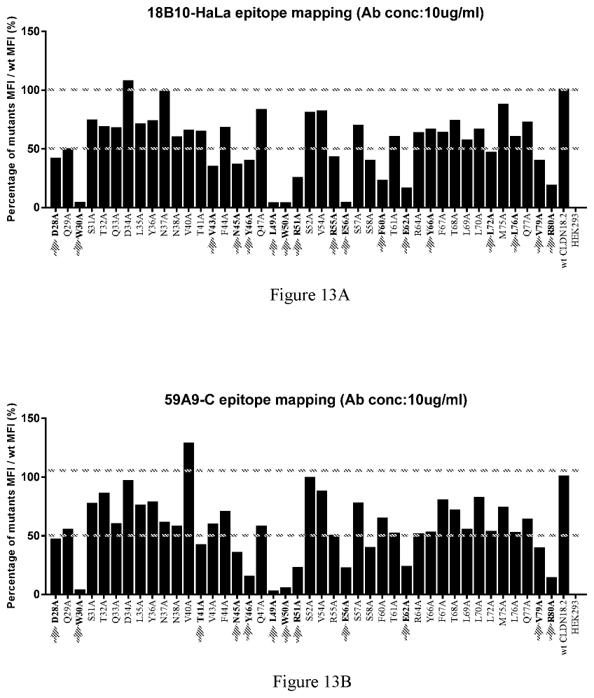

As shown in FIG. 15, 18B10-HaLa showed a slightly better anti-tumor activity than IMAB362 as measured by the tumor size and the TGI, while both were significantly better than the isotype control (Table 10).

TABLE 10
Tumor Growth Inhibition (TGI) of 18B10-HaLa and
IMAB362 in MKN45-CLDN18.2-high Xenograft Model
(mean ± S.E.M., n = 8)

| | Tumor size | | p value vs. |
|---|---|---|---|
| | (mm\hat{ }3) | TGI (%) | Isotype |
| Treatment | Day 28 | Day 28 | control |
| Isotype control | 541.16 ± 48.39 | / | / |
| IMAB362 | 423.11 ± 27.87 | −21.82 | 0.0529 |
| 18B10-HaLa | 260.31 ± 20.30 | 51.90 | 0.0001 |

2. Anti-Tumor Efficacy on MKN45-CLDN18.2-High and hPBMC Co-Inoculation Xenograft Model Using NOD-SCID Mice

Human PBMC cells were acquired from Allcells. 24 female SPF grade NOD-SCID mice were randomized to 3 μgroup (n=8), 6 mice inoculated with 5×10\hat{ }6 MKN45-CLDN18.2-high cells and 50% matri-gel (BD) by s.c. injection on the right flank as model group (without PBMC), and 18 mice were inoculated with 5×10\hat{ }6 MKN45-CLDN18.2-high cells and 5×10\hat{ }6 human PBMC cell with 50% matri-gel (BD) as treatment group. 4 hours after inoculation the mice were treated with 10 mg/kg isotype control, 3 mg/kg and 10 mg/kg 18B10-HaLa, twice a week for 4 weeks by i.p. injection. Animals were sacrificed at the end of the study with CO₂ inhalation. Tumor size and volume were measured 2-3 times a week. Results were analyzed using Prism GraphPad and expressed as mean±S.E.M.

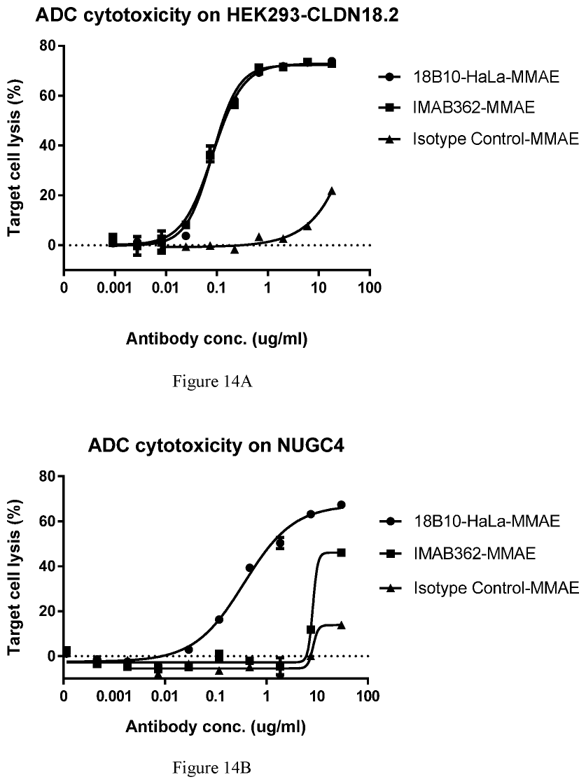

As shown in FIG. 16, the tumor growth in the 18B10-HaLa group was completely inhibited during the treatment period. Post treatment, the tumor from the 3 mg/kg group began to outgrow after 20 days, while 10 mg/kg group did not. No significant difference between the group without PBMC and the PBS group with PBMC suggested that PBMC alone as effector cells without antibody could not inhibit tumor growth. The tumor growth inhibition (TGI) was summarized in Table 11. 18B10-HaLa had no effect on animal weight (data not shown).

TABLE 11
Tumor Growth Inhibition of 18B10-HaLa in
MKN45-CLDN18.2-high and hPBMC co-inoculation
xenograft tumor model (mean ± S.E.M., n = 6)

| | Tumor size | TGI (%) | p value vs. |
|---|---|---|---|
| Treatment | (mm\hat{ }3) Day 29 | Day 29 | Isotype control |
| Model Group | 797.59 ± 98.07 | / | / |
| Isotype control | 926.27 ± 175.36 | / | / |
| 18B10-HaLa 3 mg/kg | 273.62 ± 41.16 | 70.46 | 0.0047 |
| 18B10-HaLa 10 mg/kg | 85.40 ± 10.35 | 90.78 | 0.0007 |

3. 18B10-HaLa Dose-Dependently Inhibited Tumor Growth of MKN45-CLDN18.2-High Xenograft in Nude Mice

Each female Balb/c nude mice was inoculated with 5×10\hat{ }6 cells with 50% matri-gel (BD) by s.c. injection on the right flank. 9 days after inoculation, 32 mice with tumor size around 100 mm\hat{ }3 were selected and randomized into 4 μgroups (n=8). Then the mice were treated with isotype control, 0.1 mg/kg, 0.3 mg/kg and 1 mg/kg 18B10-HaLa, twice a week for 3 weeks by i.p. injection. Animals were sacrificed at the end of the study with CO₂ inhalation. Tumor size and volume were measured 2-3 times a week. Results were analyzed using Prism GraphPad and expressed as mean S.E.M.

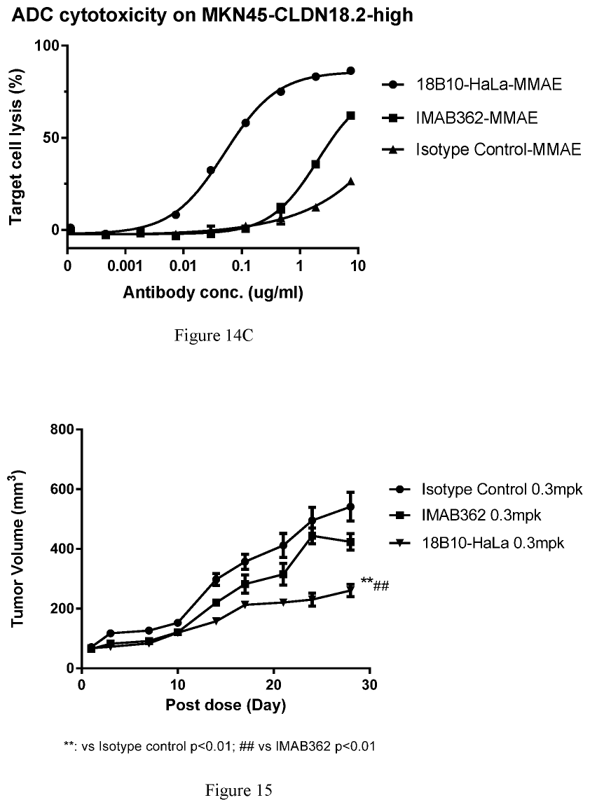

As shown in FIG. 17, the anti-tumor activity of 18B10-HaLa was dose-dependent. The 1 mg/kg group showed the best tumor growth inhibition activity (Table 12).

TABLE 12
Dose-dependent Tumor Growth Inhibition of 18B10-HaLa in MKN45-CLDN18.2-high
Xenograft Tumor Model (mean ± S.E.M., n = 8)

| | Tumor size (mm^{∧}3) | TGI (%) | p value vs. Isotype |
|---|---|---|---|
| Treatment | Day 13 | Day 13 | control |
| Isotype control | 584.37 ± 32.32 | / | / |
| 0.1 mg/kg 18B10-HaLa | 409.75 ± 44.46 | 29.88 | 0.0067 |
| 0.3 mg/kg 18B10-HaLa | 369.84 ± 19.14 | 36.71 | 5.38*10^{∧}-5 |
| 1 mg/kg 18B10-HaLa | 275.57 ± 23.41 | 52.84 | 2.02*10^{∧}-6 |

### Example 13: Generation, Expression, Purification and Characterization of 18B10-HaLa-VLPYLL Mutant with Enhanced ADCC Effect

1. Generation of 18B10-HaLa-VLPYLL Mutant

According to the study from Futa Mimoto et al., L235V/F243L/R292P/Y300L/P396L mutations could increase 10-fold binding affinity to FcγRIIIA without any change against FcγRIIB, which is an inhibitory FcγR isoform. To test this hypothesis, 18B10-HaLa-L235V/F243L/R292P/Y300L/P396L (18B10-HaLa-VLPYLL) mutant was constructed and generated to enhance its ADCC effect. This Fc variant was transient transfected, expressed and purified following the same methods as Section 1 of Example 12.

It has been reported that five mutations L235V/F243L/R292P/Y300L/P396L in Fc can increase binding affinity to both alleles of human CD16A (FcγRIIIA), without any change against FcγRIIB, which is an inhibitory FcγR isoform (Futa Mimoto et al., Novel asymmetrically engineered antibody Fc variant with superior FcγR binding affinity and specificity compared with afucosylated Fc variant[C]//MAbs. Taylor & Francis, 2013, 5(2): 229-236). To test this hypothesis, these mutations were introduced into Hu18B10_Ha_hIgG1 by using the overlap extension PCR, and the new construct is named as Hu18B10_Ha_hIgG1_L235V/F243L/R292P/Y300L/P396L. The final PCR products were characterized by agarose gel electrophoresis. The correct size fragment was extracted from gel and cloned into expression vector. The correct construct of Hu18B10_Ha_hIgG1_P330S was then confirmed by sequencing analysis. The plasmids of Hu18B10_Ha_hIgG1_L235V/F243L/R292P/Y300L/P396L and Hu18B10_La_hKappa were prepared by using the Plasmid Maxi-prep System from Qiagen. Then the heavy chain and light chain plasmids were co-transfected into Expi-CHO cell for expression and purification as previously described above following the same methods as Section 1 of Example 10.

Sequences of engineered Fc
L235V/F243L/R292P/Y300L/P396L (SEQ ID NO: 51):
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELVGGPSVFLLPPKPKDTLMISRTPEVTCVVV
DVSHEDPEVKFNWYVDGVEVHNAKTKPPEEQYNSTLRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPLVLDSDGSFFLYSKLT
VDKSRWQQGNVFSCSVMHEALHNHYTQKSLSLSPGK

2. Binding to Fc Gamma Receptors

Futa Mimoto et al had compared VLPYLL Fc mutant to the wildtype one and found the mutations could increase its binding affinity to both FcγRIIIA F176 (63-fold) and FcγRIIIA V176 (33-fold) without affecting other FcgRs. To confirm this finding, ELISA binding between antibodies and these FcγRs were tested. Briefly, 18B10-HaLa-VLPYLL or 18B10-HaLa-wt antibody was coated on the plate at a concentration of 1 μg/ml. After blocking and washing, the serial diluted (5 g/ml˜0.02 μg/ml) FcγRs labeled with His-tag were added and incubated for 1 h. Then anti-His-HRP and TMB were added for detection of FcγR binding at OD450 nm.

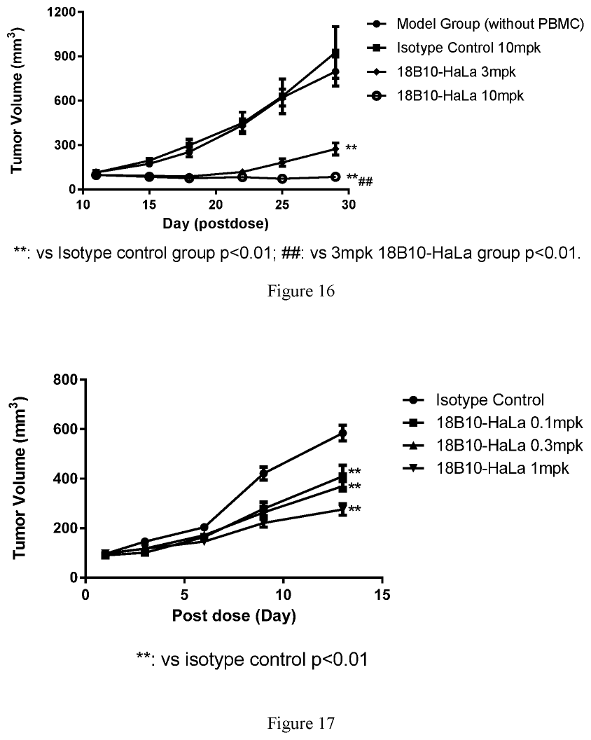

As shown in FIGS. 18A and 18B, there was no significant difference between 18B10-HaLa_VLPYLL and 18B10-HaLa-wt in binding to human FcγRI or FcγRIIB. However 18B10-HaLa_VLPYLL showed 10-fold increased binding to human FcγRIIIA (F176) and FcγRIIIA (V176) as compared to its wild-type (wt) one (FIGS. 18C and 18D). The similar results were shown with mouse FcγRs and cyno FcγRs (FIG. 18E to 18I).

3. Binding to FcRn and C1q

FcRn binding was evaluated by an ELISA method. Briefly, 18B10-HaLa_VLPYLL or wt were immobilized on the plate. Biotinylated FcRn was serial diluted in a pH6.0 dilution buffer (1 μg/ml˜0.0002 μg/ml) and then added for 1 h incubation. Next, Streptavidin-HRP and TMB were added for detection of binding at OD450 nm.

C1q binding assay was taken following method. Two antibodies were immobilized on the plate. Serial diluted C1q (20 μg/ml˜0.31 μg/ml) were added for 1 h incubation. Then anti-C1q-HRP and TMB were added for detection at OD450 nm.

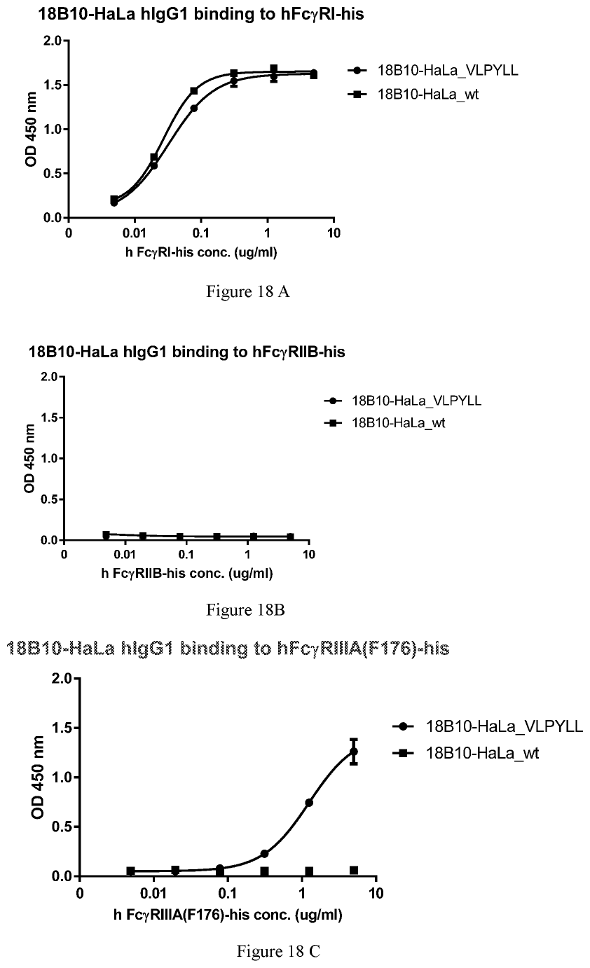

As shown in FIG. 19A, there is no significant difference in FcRn binding between 18B10-HaLa_VLPYLL and wt, indicating VLPYLL mutations had no effect on FcRn binding. FIG. 19B showed 18B10-HaLa_VLPYLL reached the same binding signal at a lower C1q concentration than that of wt, which may lead to an increased CDC potency.

4. ADCC Assay on NUGC4 Cell Using Jurkat-NFAT-Luc-FcγRIIIA-V176 as the Effector Cells

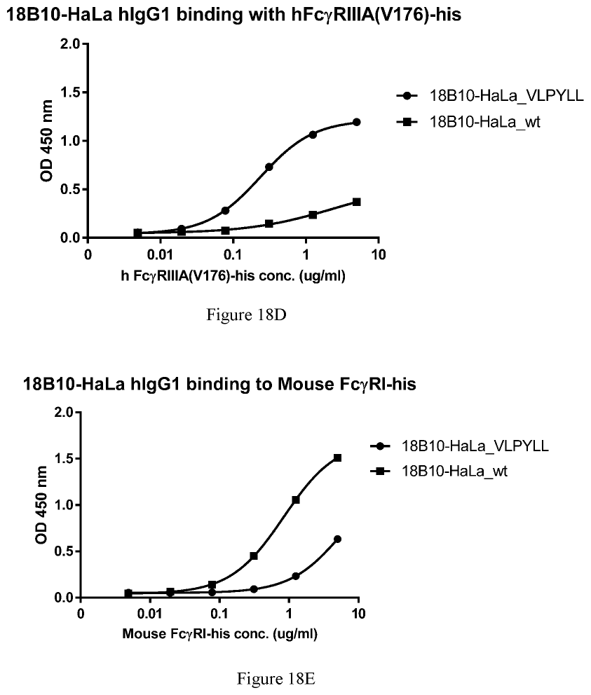

ADCC reporter assay was performed following the same method above (see Section 2 of Example 7). As shown in FIG. 20A, 18B10-HaLa_VLPYLL had a 3-fold increase in ADCC potency (EC50-0.0097 μg/ml) as compared to that of wt (EC50-0.032 μg/ml).

5. ADCC Assay on NUGC4 Cell Using Human PBMC as the Effector Cells

ADCC assay using human PBMC was performed following the method above (see Section 7 of Example 10). As shown in FIG. 20B, 18B10-HaLa_VLPYLL also had a 3-fold increase of ADCC potency as compared to that of wt, though the maximum cytotoxicity of both were similar (˜45%). When compared to IMAB362, 18B10-HaLa_VLPYLL had a 100-fold increase of potency.

6. MESF of CLDN18.2 Expression on a Panel of Gastric Cancer Cell Lines

Quantum™ MESF (Molecules of Equivalent Soluble Fluorochrome) microsphere kits enable the standardization of fluorescence intensity units for applications in quantitative fluorescence cytometry. A panel of gastric cancer (GC) cells were stained by using 30 μg/ml 18B10-HaLa and goat anti-human IgG-FITC. Cells were detected by using Quantum™ MESF beads on the flow cytometer with a fixed fluorescence setting. Briefly, add one drop of the reference blank “B” to 400 μL suspending solution, then combine 1 drop of each of the fluorescence intensity populations to 400 μL of the same buffer for analysis. The microspheres were analyzed on the flow cytometer. The downloaded Bangs Laboratories' quantitative analysis template, QuickCal® v. 2.3 was utilized for data analysis, using a calibration curve and Regression Coefficient (r2) value. For accurate MESF assignments, instrument linearity was assured, and a regression coefficient ≥0.9995 was reached. Also, appropriate controls (e.g. unstained cells, isotype controls) were run in parallel.

As shown in FIG. 21, two transfected cell lines HEK293-CLDN18.2 and MKN45-CLDN18.2-high had a much higher level of CLDN18.2 expression than other cell lines, which may not represent tumor cells from GC patients. Among the GC cell lies, NUGC4 had the highest expression of CLDN18.2. SNU-601 (Cobioer, Cat #CBP60507) and SNU-620 (Cobioer, Cat #CBP60508) had a moderate level, while KATOIII and OCUM-1 (Cobioer, Cat #CBP60494) had a lower expression. Therefore, CLDN18.2 has different expression level among gastric cancer cells.

7. IHC Detection of CLDN18.2 Expression on a Panel of Gastric Cancer Cell Lines

The gastric cancer cell lines were collected at log growth phase and fixed in 4% neutral buffered paraformaldehyde (PFA) for 30 min at room temperature after washing with phosphate-buffered saline (PBS) respectively. After centrifugation, cells re-suspended in PBS at density of 2-5×10\hat{ }7 approximately, subsequently mixed with 200 μl molten agar, followed by dehydration in gradient alcohol, clear in xylene and then embedded in paraffin wax for section. The CLDN18.2 expression level of these cell was detected via Immunohistochemistry (IHC) using 3 μg/ml GC182-Biotin, generated by Mabspace Bioscience according to the sequence in WO2013167259 and biotinylated in house, which is the available monoclonal antibody for CLDN18.2 IHC detection. IHC results were evaluated by the relative proportion of positive cells and staining intensity on cell membrane. According to the scoring guidelines of IMAB362 in clinical trial, these cell lines were scored and assessed (Table 13). Only patients with moderate (2+) and strong (3+) staining in at least 40% of tumor cells were eligible for inclusion in the FAST study of IMAB362. Therefore, NUGC4, MKN45-CLDN18.2-high and HEK293-CLDN18.2 meet the criteria. The results were consistent with that of Example 13 section 6 (Quantum™ MESF method).

Heavy chain variable region of GC182
(SEQ ID NO: 74):
QIQLVQSGPELKKFGETVKISCKASGYTFTDYSIHWVKQAPGKGLKWMG
WINTETGVPTYADDFKGRFAFSLETSASTAYLQINNLKNEDTATYFCAR
RTGFDYWGQGTTLTVSS
Light chain variable region of GC182
(SEQ ID NO: 75):
DIVMTQAAFSIPVTLGTSASISCRSSKNLLHSDGITYLYWYLQRPGQSP
QLLIYRVSNLASGVPNRFSGSESGTDFTLRISRVEAEDVGVYYCVQVLE
LPFTFGGGTKLEIK

TABLE 13
Analysis of CLDN18.2 expression of gastric cancer cell lines
via IHC method

| Cell line | Positive cell % | Intensity |
|---|---|---|
| HEK293-CLDN18.2 | 100 | 3+ |
| MKN45-CLDN18.2-high | 40~50 | 2+ |
| MKN45-CLDN18.2-medium | <5 | 0~1+ |
| NUGC4 | 30~40 | 1+~2+ |
| SNU-601 | 5~10 | 1+ |
| KATOIII | <1 | 0~1+ |
| SNU-620 | <1 | 0~1+ |
| OCUM-1 | 0 | 0 |

8. ADCC Assay on Gastric Cancer (GC) Cell Lines with a Different CLDN18.2 Expression Level Using Human PBMC as the Effector Cells

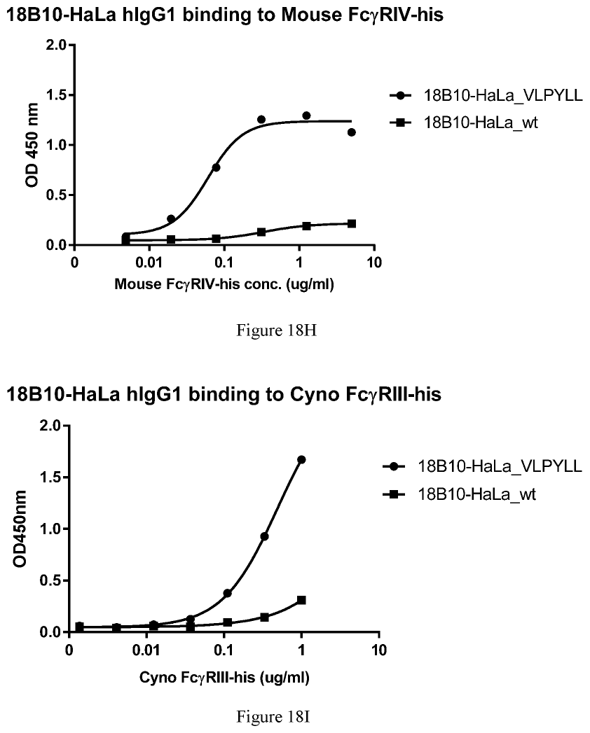

To further test the hypothesis that ADCC activity of the CLDN18.2 antibodies is regulated by the expression level of CLDN18.2 on GC cells, ADCC assay using human PBMC as the effector cells were performed following same method above (see Section 7 of Example 10). 4 μgastric cell lines with different expression level of CLDN18.2 were used as the target cells. As shown in FIG. 22A-22D, NUGC4 cell induced the highest ADCC activity (maximum cytotoxicity ˜40%) of all the CLDN18.2 antibodies. Among the three tested antibodies, 18B10-HaLa-VLPYLL showed a much better potency than 18B10-HaLa-wt and IMAB362. SNU-601 and SNU-620 cells induced a moderate ADCC activity (maximum cytotoxicity 30% and 15%, respectively), while OCUM-1 cell had the lowest cytotoxicity (below 10%). These results suggested that ADCC activity was correlated with the CLDN18.2 expression level on these cell lines.

### Example 14: Process Optimization of 18B10-HaLa and Characterization of ADCC Effect

1. Process Optimization of 18B10-HaLa

It is well known that afucosylation or defucosylation selectively and significantly increases binding affinity to FcγRIII and leads to enhanced ADCC function. The following describes the process optimizations for decreasing fucose and enhancing ADCC.

Briefly, after the seed of cell bank was recovered and cultured in CD-CHO medium (Gibco) for 3 days, cells were expanded in basal medium (Hyclone, ActiPro+4 mM Gln+1×HT) for 6 days. Then 0 (as reference sample) or 50 μM of 2F—O—F (2-Deoxy-2-fluoro-L-fucose) were added into the bioreactor and DO (dissolved oxygen) was controlled around 40%. Feed medium 1/2 (Hyclone, Cell Boost 7a, Cell Boost 7b) was added and cell suspension was harvested when the VCD (variable cell density) was below 80% or on day 13.

Antibody titers of both reference sample and 50 μM of 2F—O—F sample were measured by HPLC after cell suspension was harvested. The titer of 50 μM of 2F—O—F sample was 4.73 μg/L on day 13, which was even higher than reference sample, indicating it was not affected by 2F—O—F.

Antibody quality was measured by HPLC after purification and 50 M of 2F—O—F sample had similar purity (98.3%) to reference sample (98.2%). There was no significant impact of 2F—O—F on antibody quality.

N-Glycan was analyzed by HPLC at the same time and the result was shown in Table 14. Comparing to the reference sample, addition of 2F—O—F decreased percentage of G0F (FA2) (from 61.6% to 1.9%) and fucose (from 87.7% to 13.7%) but increased percentage of G0 (A2) (from 8.1% to 69.8%). Therefore, 50 μM of 2F—O—F was enough to control fucose below 15% and this may result in enhancement of ADCC effect. The product under this process (with 50 μM of 2F—O—F) was named as 18B10-HaLa low fucose.

TABLE 14
Analysis of N-Glycan of 18B10-HaLa samples

| | G0F | | | | | | |
|---|---|---|---|---|---|---|---|
| Sample | (FA2) % | G0(A2) % | Man5 % | G1F % | M % | F % | S % |
| Reference | 61.616 | 8.112 | 2.013 | 20.621 | 2.013 | 87.708 | 0.761 |
| 50 μM | 1.919 | 69.795 | 10.954 | 1.065 | 10.954 | 13.684 | 2.154 |
| 2F-O-F | | | | | | | |

In order to demonstrate that 18B10-HaLa low fucose enhances the affinity of effective FcγIIIa receptor while maintaining the affinity to FcRn, we compared the affinity of 18B10-HaLa low fucose and IMAB362-analog by Bio-Layer Interferometry (BLI) technique of Fortebio system. IMAB362-analog with human IgG1 isotype and normal glycosylation was taken as control.

In this study, FcγRI, FcγRIIa-H167, FcγRIIa-R167, FcγRIIb, FcγRIIIa-V176, FcγRIIIa-F176, FcγRIIIb-NA1, FcγRIIIb-NA2 and FcRn were loaded on the biosensors and dipped into IMAB362-analog and 18B10-HaLa low fucose in solution with varying concentrations. All binding data were collected at 30° C. When measuring the affinity of 18B10-HaLa low fucose or IMAB362-analog to C1q, the biotinylated antibodies were loaded on biosensors, and then incubated with C1q in solution. When the affinity of antibodies to FcRn was measured by BLI, pH was 6.0, and pH was 7.4 for the other Fc receptors' binding assay. The experiments comprised 5 steps: 1. Baseline acquisition; 2. Human Fc gamma Receptors loading onto biosensor; 3. Second baseline acquisition; 4. Association of 18B10-HaLa low fucose and IMAB362-analog for the measurement of kₒₙ; and 5. Dissociation of antibodies for the measurement of koff. 18B10-HaLa low fucose and IMAB362-analog have similar affinity to FcγRI, FcRn or C1q, while 18B10-HaLa low fucose shows slightly higher affinity than IMAB362-analog for other receptors. These results indicate that 18B10-HaLa low fucose will exhibit enhanced ADCC activity and a similar half-life with normal glycosylated antibodies in clinical trials.

TABLE 15
Data of affinity of 18B10-HaLa low fucose and IMAB362-analog to human Fc receptor detected
by ForteBio Octet

| Ligand | Analyte | kon (1/Ms) | kdis (1/s) | K_{D} (M) |
|---|---|---|---|---|
| FcγRI | IMAB362-analog | 6.67E+04 | 3.48E−04 | 5.22E−09 |
| | 18B10-HaLa low fucose | 8.49E+04 | 4.46E−04 | 5.25E−09 |
| FcγRIIa-H167 | IMAB362-analog | 1.97E+05 | 3.81E−02 | 1.93E−07 |
| | 18B10-HaLa low fucose | 3.38E+05 | 2.98E−02 | 8.81E−08 |
| FcγRIIa-R167 | IMAB362-analog | 2.55E+05 | 2.97E−02 | 1.17E−07 |
| | 18B10-HaLa low fucose | 4.04E+05 | 2.03E−02 | 5.02E−08 |
| FcγRIIb | IMAB362-analog | 1.60E+05 | 3.67E−02 | 2.30E−07 |
| | 18B10-HaLa low fucose | 3.54E+05 | 3.29E−02 | 9.30E−08 |
| FcγRIIIa-V176 | IMAB362-analog | 1.59E+05 | 9.75E−03 | 6.15E−08 |
| | 18B10-HaLa low fucose | 3.59E+05 | 9.46E−03 | 2.64E−08 |
| FcγRIIIa-F176 | IMAB362-analog | 1.42E+05 | 3.28E−02 | 2.32E−07 |
| | 18B10-HaLa low fucose | 4.07E+05 | 3.88E−02 | 9.53E−08 |
| FcγRIIIb-NA1 | IMAB362-analog | 1.05E+04 | 4.18E−02 | 3.97E−06 |
| | 18B10-HaLa low fucose | 2.53E+04 | 5.47E−02 | 2.17E−06 |
| FcγRIIIb-NA2 | IMAB362-analog | 1.40E+04 | 4.43E−02 | 3.16E−06 |
| | 18B10-HaLa low fucose | 3.52E+04 | 4.07E−02 | 1.16E−06 |
| FcRn | IMAB362-analog | 4.46E+05 | 4.67E−03 | 1.05E−08 |
| | 18B10-HaLa low fucose | 6.59E+05 | 5.49E−03 | 8.34E−09 |
| IMAB362-analog | C1q | 6.87E+06 | 1.48E−01 | 2.15E−08 |
| 18B10-HaLa low fucose | | 7.96E+06 | 1.37E−01 | 1.72E−08 |

As shown in Table 15, the affinities of 18B10-HaLa low fucose to human FcγRIIIa-V176 and human FcγRIIIa-F176 protein were a little higher than those of IMAB362, which may be caused by lower fucosylation. As shown in Table 15, the affinity of 18B10-HaLa low fucose to human FcRn protein was not effected by lower fucosylation, even a little higher than that of IMAB362. As shown in Table 15, the affinity of 18B10-HaLa low fucose to human C1q protein was not quite similar to that of IMAB362.

2. ADCC Reporter Assay on NUGC4 Using Jurkat-NFAT-Luc-FcγRIIIA-V176 as the Effector Cells

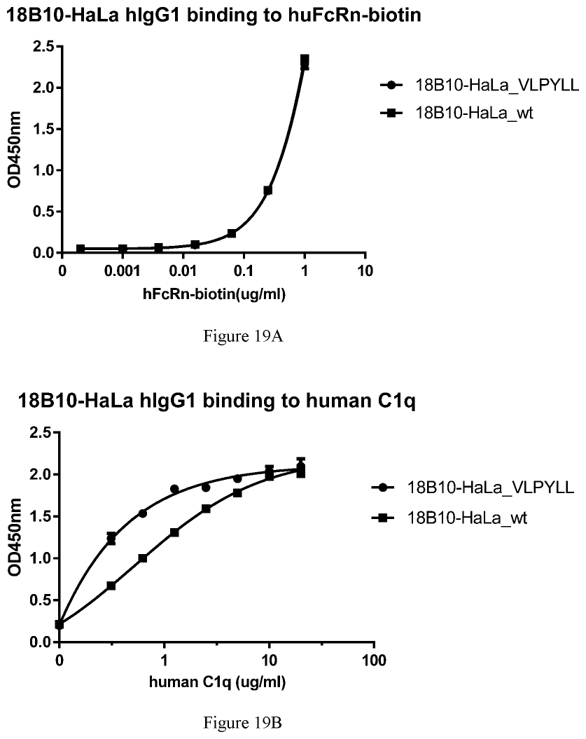

ADCC test was carried out following the same protocol above (see Section 2 of Example 7). As shown in FIG. 23, the antibody produced using the process with the addition of 50 μM 2F—O—F (18B10-HaLa low fucose) increased the ADCC activity by over 30-fold than that of reference sample produced using a process without the addition of 2F—O—F. Transient expressed 18B10-HaLa was also included in this comparison and due to process optimization, the maximum signal was also increased, which represents ADCC activity.

3. FACS Binding to Different Gastric Cancer Cell Lines Using 18B10-HaLa Low Fucose

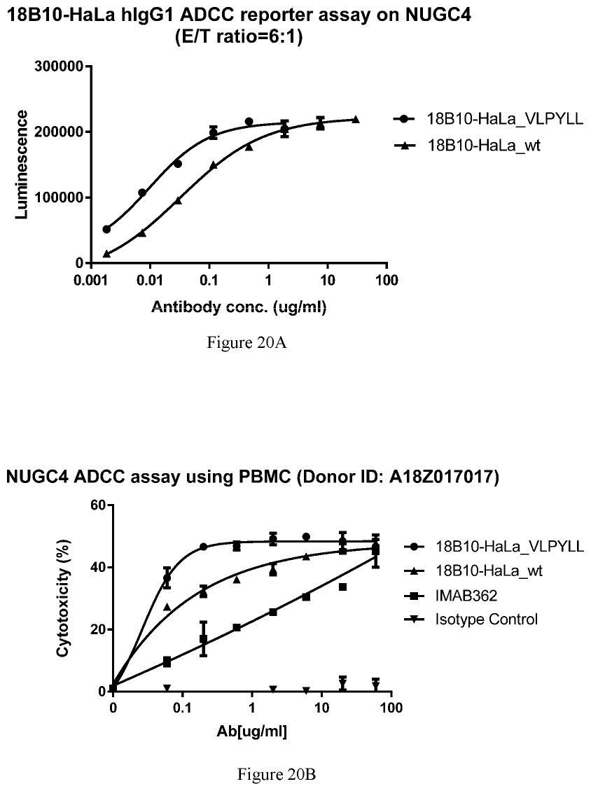

FACS binding was carried out following the same protocol of Section 2 of Example 7. As shown in FIG. 24A-24C, 18B10-HaLa low fucose could bind to these cell lines with higher potency than that of IMAB362. The EC50 of 18B10-HaLa low fucose was 0.5-1.6 μg/ml while IMAB362 nearly had no binding signal around the concentration of 1 μg/ml.

4. ADCC Reporter Assay on Different Gastric Cancer Cell Lines Using Jurkat-NFAT-Luc-FcγRIIIA-V176 as the Effector Cells

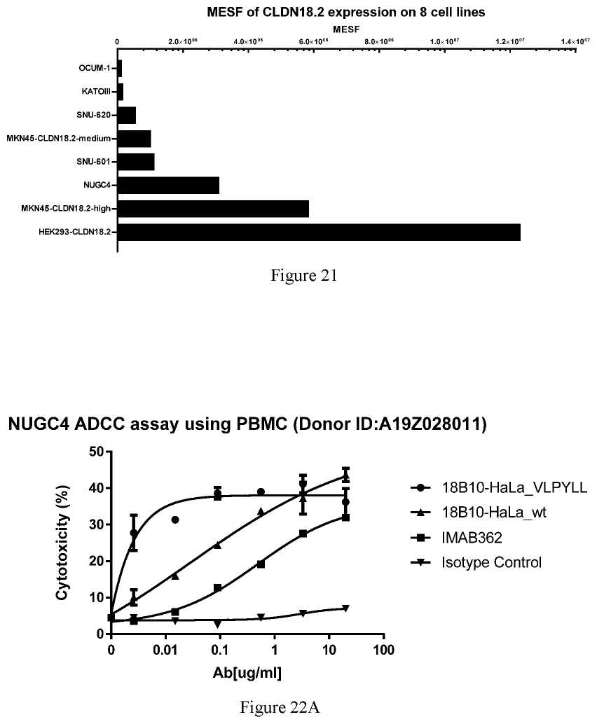

ADCC test was carried out following the same protocol above (see Section 2 of Example 7). As shown in FIG. 25A-E, the EC50 of ADCC activity of 18B10-HaLa low fucose was around 0.008 μg/ml using gastric cancer cell lines with different levels of CLDN18.2 expression. Comparing to IMAB362, 18B10-HaLa low fucose had at least 100-fold higher ADCC potency.

5. ADCC Reporter Assay on Different Gastric Cancer Cell Lines Using PBMC as the Effector Cells

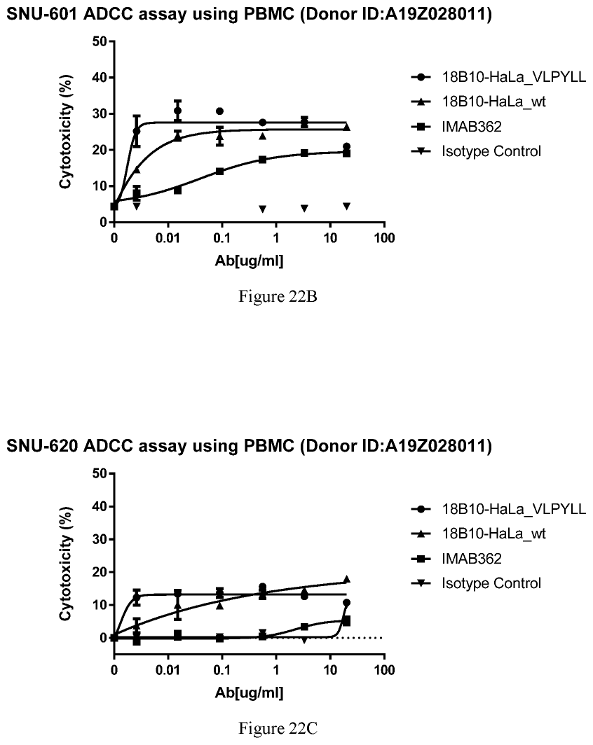

ADCC test was carried out following the same protocol above (see Section 7 of Example 10). As shown in FIG. 26A-26D, 18B10-HaLa low fucose significantly induced higher ADCC effect than IMAB362 on different gastric cancer cell lines. IMAB362 hardly induced any cytotoxicity at low concentration (0.01˜0.1 g/ml). However, at concentration of 0.1 μg/ml, the cytotoxicity of 18B10-HaLa low fucose was nearly saturated.

6. Optimized ADCC Assay on NUGC4 Using PBMC as the Effector Cells

Optimized ADCC assay using human PBMC as effector cells was developed for further study. Briefly, recover the frozen PBMC from liquid nitrogen and resuspend cells with RPMI1640+10% FBS at density of 5×10\hat{ }6/ml and incubate them in a 37° C. 5% CO₂ incubator for 5 h before use. Label the target cell NUGC4 cells with CellTrace™ Far Red (Invitrogen, cat #C34564) following the instruction. Add the labeled NUGC4 cells and diluted antibody into 96-well plate and incubate them in a 37° C. 5% CO₂ incubator for 30 minutes. Then add PBMC cells into corresponding wells and incubate cells in the incubator for 15 hours. At the end of culture, add Propidium Iodide (PI) Staining Solution to mark dead NUGC4 cells. Analyze PI positive cell percentage in CellTrace™ Far Red positive cells by flow cytometry. Specific cytotoxicity was calculated by subtracting non-specific killing percentage.

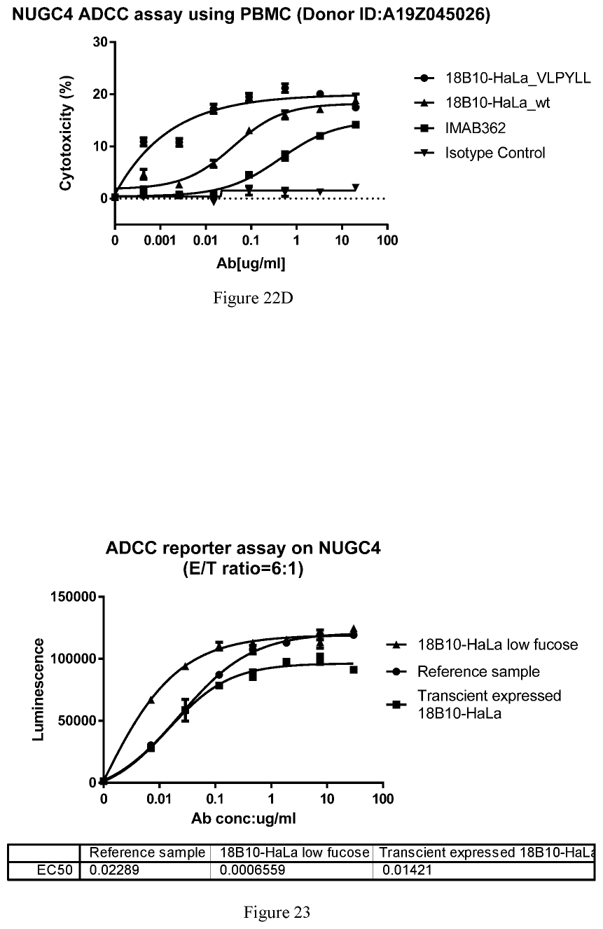

As shown in FIG. 27, a representative data, the maximum specific cytotoxicity of 18B10-HaLa low fucose reached over 60% at the concentration of 1.2 μg/ml and its EC50 was 0.014 μg/ml, while the maximum of IMAB362-analog was only 40% at the highest concentration (30 μg/ml) and its EC50 was 0.54 μg/ml, which is over 30 times that of 18B10-HaLa low fucose.

### Example 15: Anti-Tumor Activity of 18B10-HaLa Low Fucose in Vivo

1. Anti-Tumor Efficacy on MKN45-CLDN18.2-High and hPBMC Co-Inoculation Xenograft Model Using NOD-SCID Mice

Human PBMC cells were acquired from Allcells. 60 female SPF grade NOD-SCID mice were randomized to 6 μgroup (n=10), 10 mice inoculated with 5×10\hat{ }6 MKN45-CLDN18.2-high cells and 50% matri-gel (BD) by s.c. injection on the right flank as model group (without PBMC), and 50 mice were inoculated with 5×10\hat{ }6 MKN45-CLDN18.2-high cells and 5×10\hat{ }6 human PBMC cell with 50% matri-gel (BD) as treatment group. 4 hours after inoculation the mice were treated with 10 mg/kg isotype control, 1 mg/kg, 3 mg/kg and 10 mg/kg 18B10-HaLa low fucose, twice a week for 5 weeks by i.p. injection. Animals were sacrificed at the end of the study with CO₂ inhalation. Tumor size and volume were measured 2-3 times a week. Results were analyzed using Prism GraphPad and expressed as mean±S.E.M.

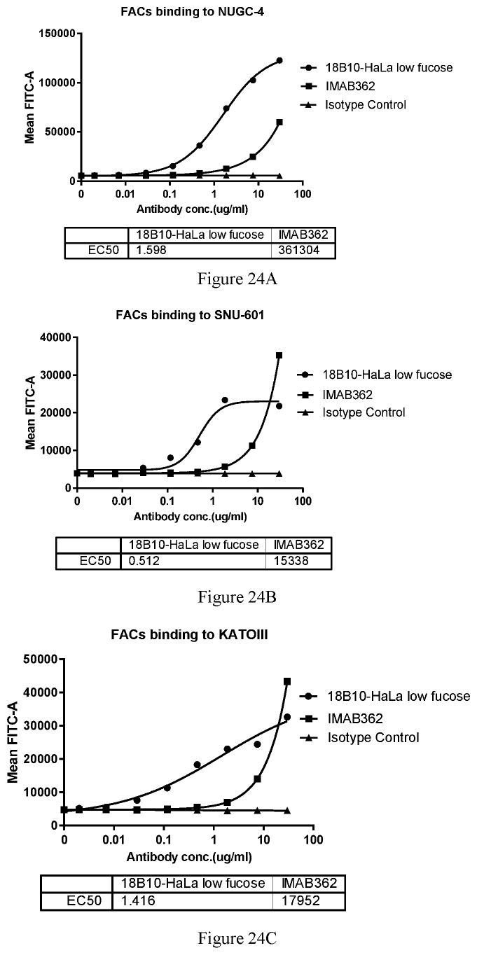

As shown in FIG. 28A, the tumor growth was significantly inhibited by 18B10-HaLa low fucose in a dose-dependent manner. Especially with 10 mg/kg of 18B10-HaLa low fucose, most of tumors (7/10) disappeared at the end of study (FIG. 28B). The tumor growth inhibition rate of 18B10-HaLa low fucose was also dependent on dosage and the TGI of 10 mg/kg group reached 95.86% (Table 16). Comparing to IMAB362-analog, 1810-HaLa low fucose has much more potent anti-tumor activity. Meanwhile 18B10-HaLa low fucose had no effect on animal weight (data not shown).

TABLE 16
Tumor Growth Inhibition of antibodies in MKN45-CLDN18.2-high and hPBMC co-inoculation
xenograft tumor model on Day 36 (mean ± S.E.M., n = 10)

| | Tumor size | | p value vs. |
|---|---|---|---|
| Treatment | (±SEM, mm^{∧}3) | TGI (%) | Isotype control |
| Model Group | 953.02 ± 84.3 | / | / |
| Isotype control 10 mg/kg | 932.88 ± 118.05 | / | / |
| 18B10-HaLa low fucose 1 mg/kg | 608.2 ± 102.07 | 35.80 | 0.0520 |
| 18B10-HaLa low fucose 3 mg/kg | 279.34 ± 78.07 | 70.06 | 0.0002 |
| 18B10-HaLa low fucose 10 mg/kg | 38.62 ± 24.06 | 95.86 | 7 × 10^{∧}-7 |
| IMAB362-analog 10 mg/kg | 162.78 ± 40.6 | 82.55 | 8 × 10^{∧}-6 |

2. Efficacy of 18B10-HaLa Low Fucose Combined with Oxaliplatin and 5-Fu on MKN45-CLDN18.2-High Tumor Model in Nude Mice

Female SPF grade nude mice were inoculated with mixed 5×10\hat{ }6 MKN45-CLDN18.2-high cells with 50% matri-gel. When the tumor size around 90 mm\hat{ }3, tumor bearing mice were selected and randomized to 4 μgroups (n=8). Animals were treated with 10 mg/kg isotype control and vehicle, 10 mg/kg 18B10-HaLa low fucose, 2.5 mg/kg Oxaliplatin and 30 mg/kg 5-FU, and 10 mg/kg 18B10-HaLa low fucose combined with 2.5 mg/kg Oxaliplatin and 30 mg/kg 5-FU, 18B10-HaLa low fucose was administrated twice a week for 4 weeks by i.p. injection, while Oxaliplatin and 5 FU were administrated once a week for 4 weeks by i.v. injection. Tumor size was measured twice or triple times a week in two dimensions using a caliper (INSIZE) and the volume was expressed in mm\hat{ }3 using the formula: V=0.5 a×b\hat{ }2 where a and b ate the long and shirt diameters of the tumor, respectively. Results were analyzed using Prism GraphPad and expressed as mean±S.E.M. Comparisons between two groups were made by T-test, and the difference is considered significant if p is *<0.05 and **<0.01.

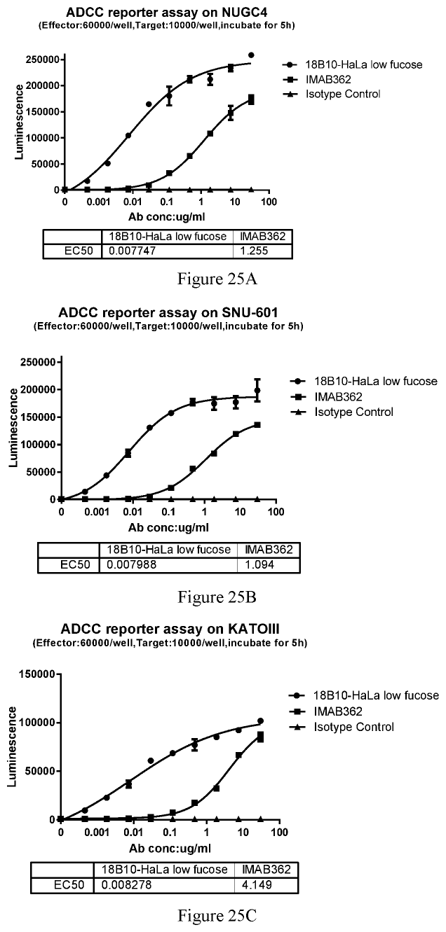

As shown in FIG. 29 and Table 17, without PBMC, single agent groups of 18B10-HaLa low fucose and Oxaliplatin+5-FU only had mild inhibition of tumor growth with TGI of 47% and 52% respectively. But the combination of them had an enhanced tumor inhibition 69%, with significant difference compared with single agent groups.

TABLE 17
Tumor Growth Inhibition of 18B10-HaLa low fucose combination with Oxaliplatin and 5-FU
on MKN45-CLDN18.2-high tumor model on Day 28 (mean ± S.E.M., n = 8)

| | Tumor size | | p value vs. |
|---|---|---|---|
| Treatment | (±SEM, mm^{∧}3) | TGI (%) | Isotype control |
| 10 mg/kg Isotype control + vehicle | 1617.77 ± 66.37 | / | / |
| 18B10-HaLa low fucose 10 mg/kg | 852.28 ± 75.68 | 47.32 | 3 × 10^{∧}- 6 |
| Oxaliplatin 2.5 mg/kg + 5-FU 30 mg/kg | 770.66 ± 87.38 | 52.36 | 2 × 10^{∧} - 6 |
| 18B10-HaLa low fucose 10 mg/kg + | 508.03 ± 77.02 | 68.60 | 3 × 10^{∧}- 8 |
| (Oxaliplatin 2.5 mg/kg + 5-FU 30 mg/kg) | | | |

3. Efficacy of 18B10-HaLa Low Fucose Combined with Paclitaxel on MKN45-CLDN18.2-High Tumor Model in Nude Mice

MKN45-CLDN18.2-high cells were maintained in vitro as a monolayer culture in RPMI1640 medium (Thermo Fisher) supplemented with 10% heat inactivated fetal bovine serum (ExCell Biology), 100 U/ml penicillin and 100 ug/ml streptomycin (Hyclone) at 37° C. with 5% C02. Cells in an exponential growth phase were harvested and counted for tumor inoculation. Each female Balb/c nude mice was inoculated with 5×10\hat{ }6 cells with 50% matri-gel (BD) by s.c. injection on the right flank. 8-11 days after inoculation, 24 mice with tumor size around 100 mm\hat{ }3 were selected and randomized into 3 μgroups (n=8). Then the mice were treated with isotype control or 18B10-HaLa low fucose at dose of 10 mg/kg, twice a week for 3 weeks by i.p. injection. 5 mg/kg of Paclitaxel was i.v. injected once a week. Animals were sacrificed at the end of the study with C02 inhalation. Tumor size was measured in two dimensions using a caliper (INSIZE) and the volume was expressed in mm3 using the formula: V=0.5 a×b2 (where a and b represent the length and width of the tumor, respectively). Tumor growth inhibition rate (TGI %) was calculated using the formula: TGI %=(1−(TVDt (treatment group)/TVDt (control group))×100%. TVDt represents the tumor volume at each subsequent measurement. Histograms were generated using Prism GraphPad (mean±S.E.M.), and T analysis was used for statistical analysis. p<0.05, represents a significant difference between groups; p<0.01, represents a highly significant difference between groups.

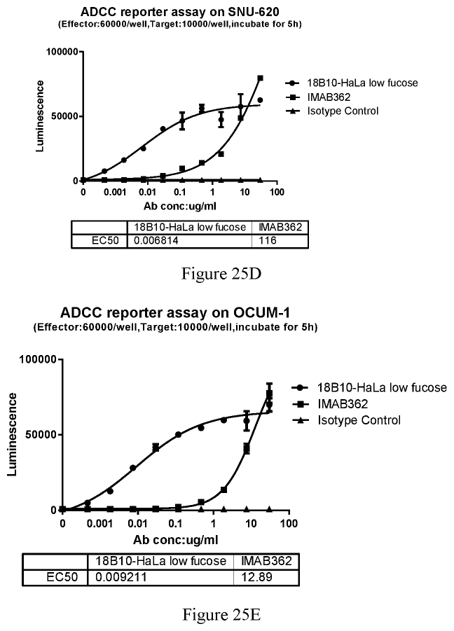

As shown in FIG. 30A, comparing with isotype control, 18B10-HaLa low fucose significantly inhibit tumor growth from Day 5 and its TGI was about 43%. Similarly, Paclitaxel, as a commonly used second line chemotherapeutic agent for gastric cancer, also had about 45% TGI. But when they were combined, tumor inhibition rate reached 61% with significant difference to single agent groups (FIG. 30B, Table 18). However, without human PBMC inoculated with tumor, tumor volumes were significantly larger than those with human PBMC. No significant body weight change was seen in all groups.

TABLE 18
Tumor Growth Inhibition of antibodies in MKN45-CLDN18.2-high xenograft tumor model
on Day 29 (mean ± S.E.M., n = 10)

| | Tumor size | | p value vs. |
|---|---|---|---|
| Treatment | (±SEM, mm^{∧}3) | TGI (%) | Isotype control |
| Isotype control 10 mg/kg | 941.20 ± 122.34 | / | / |
| 18B10-HaLa low fucose 10 mg/kg | 536.99 ± 29.26 | 42.95 | 0.0011 |
| Paclitaxel 3 mg/kg | 515.47 ± 71.93 | 45.23 | 0.0022 |
| 18B10-HaLa low fucose 10 mg/kg + | 366.55 ± 26.37 | 61.05 | 0.000045 |
| Paclitaxel 3 mg/kg | | | |

4. Efficacy of 18B10-HaLa Low Fucose Combined with Paclitaxel in GC02-0004 PDX Tumor Model in Nude Mice

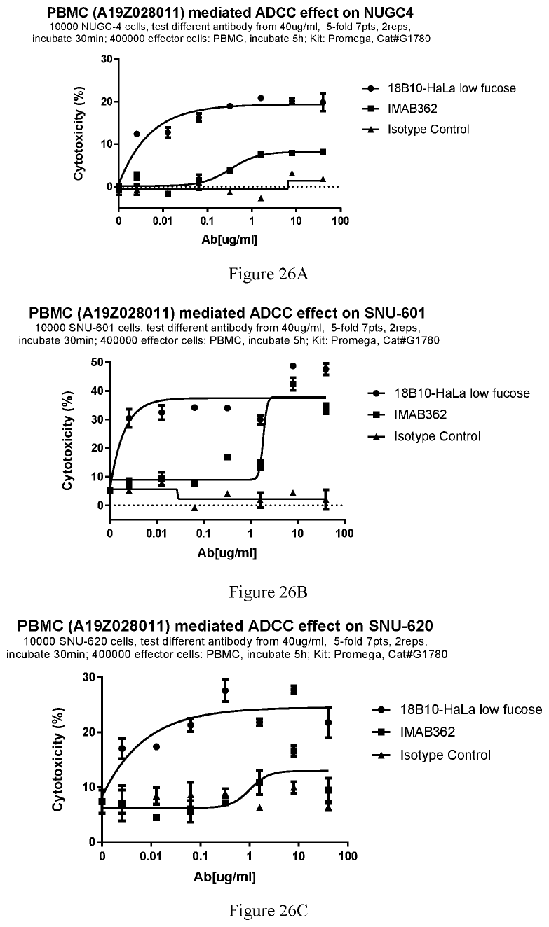

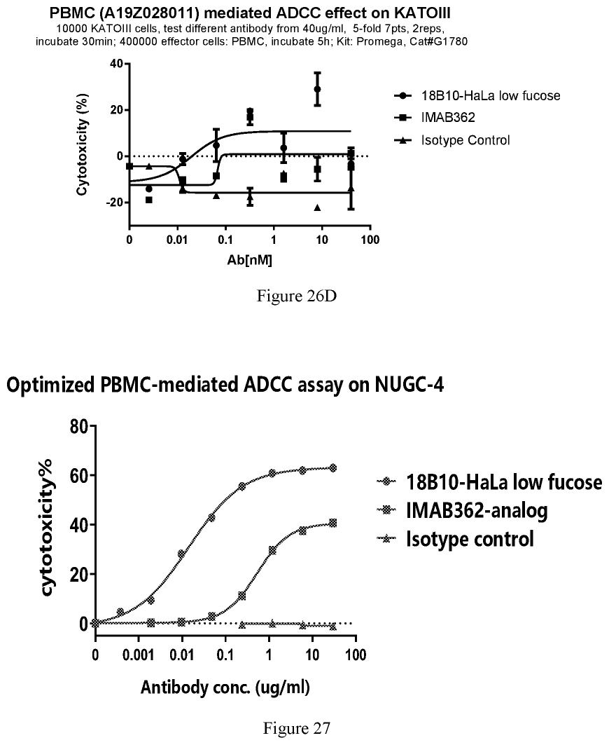

The tumor tissue of gastric cancer patients derived xenograft (PDX) model was derived from an adenocarcinoma/gastric cancer patient (No: GC-02-004) of Beijing Cancer Hospital and analyzed after 6 passages in nude mice. The CLDN18.2 expression was detected via Immunohistochemistry (IHC) using 3 μg/ml GC182-Biotin, which is the accepted IHC antibody for CLDN18.2 detection. GC182 was generated by Mabspace Bioscience according to the sequence in WO2013167259. The relative proportion of positive cells in this tumor tissue was between 40% and 70% (FIG. 31A, 200× magnification). HER2 and PD-L1 expression were also detected via IHC using Rabbit mAb D8F12 (Cell Signaling Technology, Cat #4290) and SP263 (produced by MabSpace Bioscience according to the sequence in WO2016124558), respectively. As a result, this tumor tissue was both HER2 negative (FIG. 31B) and PD-L1 negative (FIG. 31C).

Each mouse was subcutaneously inoculated with a small tumor tissue block approximately 3 mm in diameter which sheared from integrated tumor decollement form a tumor bearing mouse. 2 weeks after inoculation, animals with tumor size at about 50 mm\hat{ }3 were selected and randomly divided into 3 μgroups, each group consisting of 8 mice. 18B10-HaLa low fucose and control antibody were injected intraperitoneal 10 mg/kg twice a week. 5 mg/kg of Paclitaxel was i.v. injected once a week. Treatment continued for 5 weeks after the first injection. The tumor volume and mouse weight were measured 2-3 times per week. Animals were sacrificed at the end of the study with CO₂ inhalation. Tumor size was measured in two dimensions using a caliper (INSIZE) and the volume was expressed in mm3 using the formula: V=0.5 a×b² (where a and b represent the length and width of the tumor, respectively). Tumor growth inhibition rate (TGI %) was calculated using the formula: TGI %=(1−(TV_{Dt} (treatment group)/TV_{Dt} (control group))×100%. TV_{Dt} represents the tumor volume at each subsequent measurement. Histograms were generated using Prism GraphPad (mean±S.E.M.), and T analysis was used for statistical analysis. p<0.05, represents a significant difference between groups; p<0.01, represents a highly significant difference between groups.

As shown in FIG. 31D, 18B10-HaLa low fucose led to 48% tumor inhibition rate. Similarly, Paclitaxel, as a commonly used second line chemotherapeutic agent for gastric cancer, also had only 45% tumor inhibition rate. But when they were combined, tumor inhibition rate reached 68% with significant difference to single agent groups (Table 19). As shown in FIG. 31E, 5 mg/kg of Paclitaxel seemed to have slight tox effect on body weight of mice while other treatments did not.

TABLE 19
Tumor Growth Inhibition of antibodies in GC02-0004 PDX tumor model on Day 36
(mean ± S.E.M., n = 10)

| | Tumor size | | p value vs. |
|---|---|---|---|
| Treatment | (±SEM, mm^{∧}3) | TGI (%) | Isotype control |
| Isotype control 10 mg/kg | 1481.05 ± 304.09 | / | / |
| 18B10-HaLa low fucose 10 mg/kg | 767.65 ± 105.40 | 48.17 | 0.0510 |
| Paclitaxel 5 mg/kg | 812.71 ± 122.04 | 45.13 | 0.0687 |
| 18B10-HaLa low fucose 10 mg/kg + | 474.07 ± 74.58 | 67.99 | 0.0092 |
| Paclitaxel 5 mg/kg | | | |

5. Combination with DC101 in MKN45-CLDN18.2-High Xenograft Tumor Model

DC101 is a monoclonal antibody reacting with mouse VEGFR-2 (vascular endothelial growth factor receptor 2), also known as CD309, KDR and Flk-1. VEGFR-2 is a member of the tyrosine protein kinase family. Upon binding to its ligand VEGF, VEGFR-2 plays key roles in vascular development and permeability. DC101 was proved to competitively block the binding of VEGF and VEGFR-2, leading to reduced density of tumor microvessels and tumor growth. This antibody was produced by MabSpace Biosciences (Suzhou) Co., Limited according to the sequence in U.S. Pat. No. 5,840,301.

Female SPF grade nude mice were inoculated with mixed 5×10\hat{ }6 MKN45-CLDN18.2-high cells with 50% matri-gel. When the tumor size around 90 mm\hat{ }3, tumor bearing mice were selected and randomized to 4 μgroups (n=8). Animals were treated with 10 mg/kg isotype control, 10 mg/kg 18B10-HaLa low fucose, 3 mg/kg DC101, and 10 mg/kg 18B10-HaLa low fucose combined with 3 mg/kg DC101. All the antibodies were administrated twice a week for 4 weeks by i.p. injection. Tumor size was measured twice or triple times a week in two dimensions using a caliper (INSIZE) and the volume was expressed in mm\hat{ }3 using the formula: V=0.5 a×b\hat{ }2 where a and b ate the long and shirt diameters of the tumor, respectively. Results were analyzed using Prism GraphPad and expressed as mean±S.E.M. Comparisons between two groups were made by T-test, and the difference is considered significant if p is *<0.05 and **<0.01.

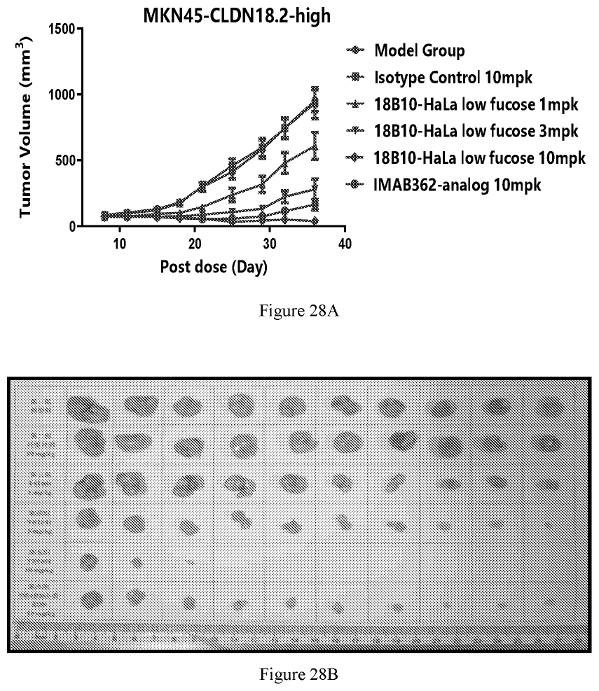

As shown in FIG. 32 and Table 20, without PBMC, single agent groups (18B10-HaLa low fucose or DC101) had some degree of tumor inhibition, with TGI of 47% and 35% respectively. When they are combined, the tumor growth almost stopped, with inhibition rate of 75%, which is significant different compared with single agent groups.

TABLE 20
Tumor Growth Inhibition (TGI) of antibodies in MKN45-CLDN18.2 Xenograft Model on
Day 22 (mean ± S.E.M., n = 6)

| | Tumor size | | p value vs. |
|---|---|---|---|
| Treatment | (±SEM, mm^{∧}3) | TGI (%) | Isotype Control |
| Isotype Control 10 mg/kg | 1141.33 ± 70.17 | | |
| 18B10-HaLa low fucose 10 mg/kg | 605.40 ± 49.96 | 46.96 | 2 × 10^{∧}-5 |
| DC101 3 mg/kg | 741.10 ± 79.96 | 35.07 | 2 × 10^{∧}-3 |
| 18B10-HaLa low fucose 10 mg/kg + | 290.57 ± 56.35 | 74.54 | 3 × 10^{∧}-7 |
| DC101 3 mg/kg | | | |

### Example 18: Anti-Tumor Activity of 18B10-HaLa Low Fucose on Pancreatic Cancer Cells In Vitro

1. Generation of MIA PaCa-2-CLDN18.2 and BxPC-3-CLDN18.2 Cell Lines

MIA PaCa-2-CLDN18.2 and BxPC-3-CLDN18.2 cell lines were constructed by MabSpace Biosciences (Suzhou) Co., Limited. Briefly, MIA PaCa-2 cell (Shanghai Institutes for Biological Sciences, Cat #SCSP-568) and BxPC-3 cell (Shanghai Institutes for Biological Sciences, Cat #TCHu12) was transfected with pcDNA3.1/hCLDN18.2 plasmids, and selected with G418 to obtain stable expressing cell line MIA PaCa-2-CLDN18.2 and BxPC-3-CLDN18.2. The expression level of CLDN18.2 was detected by 18B10-HaLa low fucose antibody. The single cell clone with a highest signal was selected and amplified for cell banking.

2. FACS Binding to Pancreatic Cancer Cell Lines Using 18B10-HaLa Low Fucose

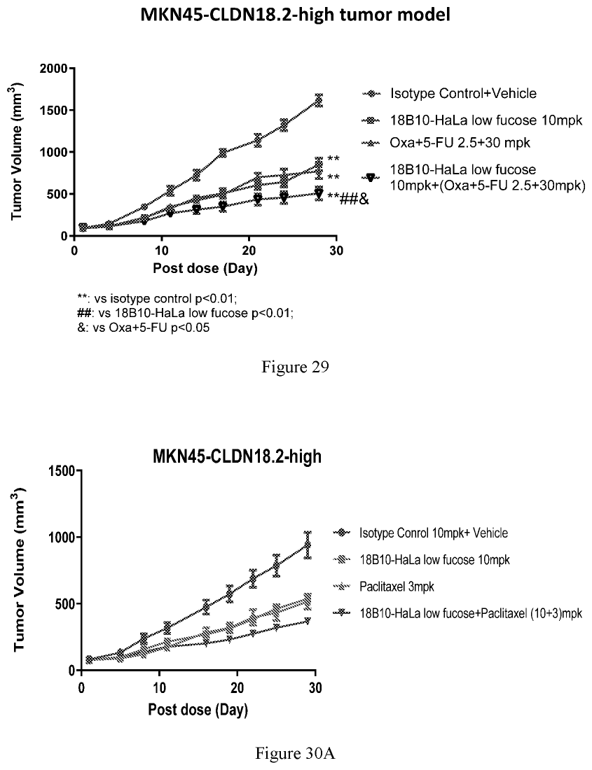

FACS binding was carried out following the same protocol of Section 2 of Example 7. As shown in FIG. 33A-33B, 18B10-HaLa low fucose could bind to the two cell lines with higher potency than that of IMAB362. The maximum signal of 18B10-HaLa low fucose was significantly higher than that of IMAB362. The EC50 of 18B10-HaLa low fucose was about 0.53 μg/ml, which was also significantly lower than that of IMAB362.

3. ADCC Reporter Assay on Pancreatic Cancer Cell Lines Using Jurkat-NFAT-Luc-FcγRIIIA-V176 as the Effector Cells

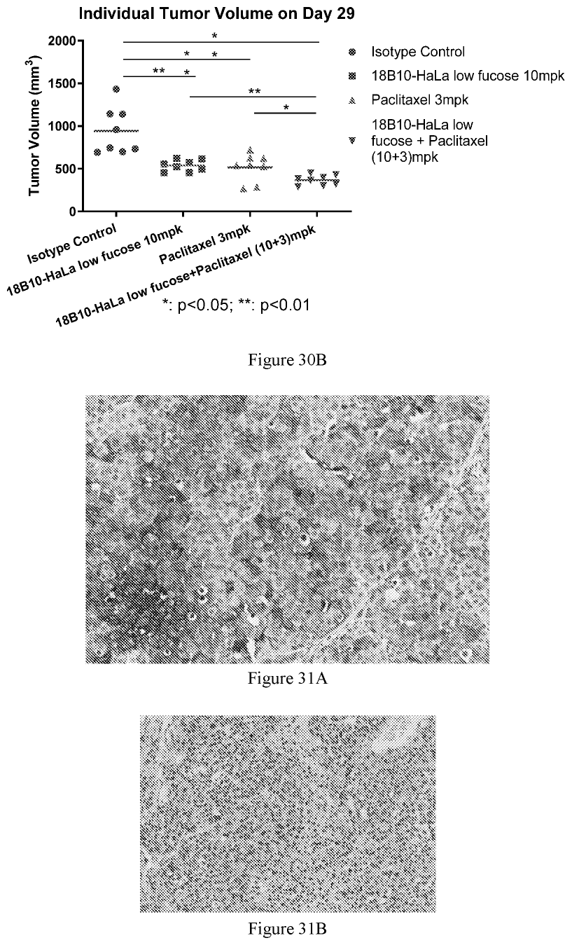

ADCC test was carried out following the same protocol above (see Section 2 of Example 7). As shown in FIG. 34A-34B, the EC50 of ADCC activity of 18B10-HaLa low fucose was around 0.001 μg/ml. Comparing to IMAB362, 18B10-HaLa low fucose had about 4-fold higher ADCC potency.

### Example 19: Anti-Tumor Activity of 18B10-HaLa Low Fucose on Pancreatic Cancer Cells In Vivo

1. Efficacy on MIA PaCa-2-CLDN18.2 Xenograft Model Using Nude Mice

Each 5-6 weeks female Balb/c nude mice was inoculated with 5×10\hat{ }6 MIA PaCa-2-CLDN18.2 cells with 50% matri-gel (BD) by s.c. injection on the right flank. 12 days after inoculation, 24 mice with tumor size around 70 mm\hat{ }3 were selected and randomized into 4 μgroups (n=6). Then the mice were treated with isotype control or 18B10-HaLa low fucose or IMAB362 at a dose of 10 mg/kg, or PBS of same volume twice a week for 5 weeks by i.p. injection. Animals were sacrificed at the end of the study with C02 inhalation. Tumor size and volume were measured 2-3 times a week. Results were analyzed using Prism GraphPad and expressed as mean±S.E.M.

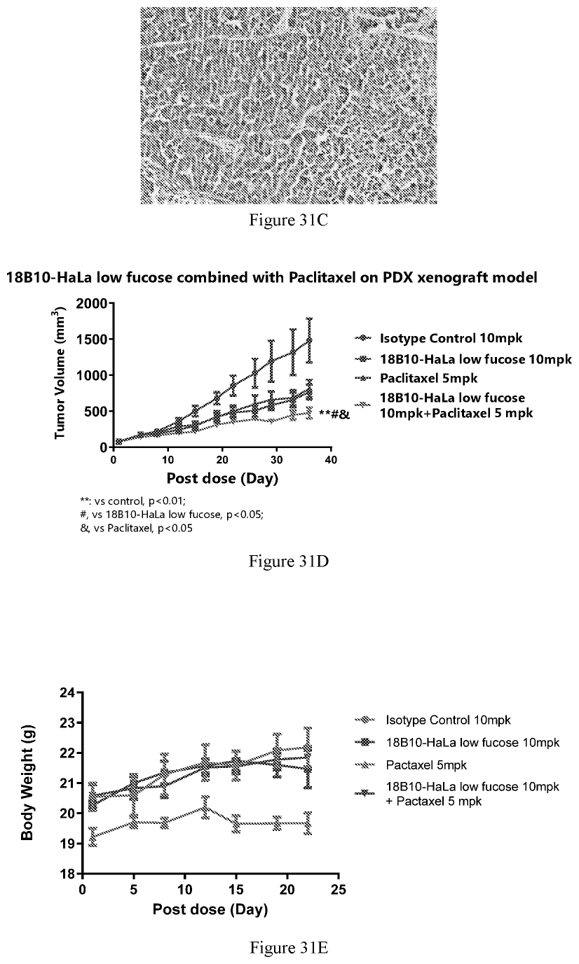

As shown in FIG. 35, 18B10-HaLa low fucose showed a better anti-tumor activity than IMAB362 as measured by the tumor size and the TGI, while both were significantly better than the isotype control (Table 21). No significant body weight change was seen in all groups.

TABLE 21
Tumor Growth Inhibition (TGI) of antibodies in MIA PaCa-2-CLDN18.2 Xenograft Model
on Day 36 (mean ± S.E.M., n = 6)

| | Tumor size | | p value vs. |
|---|---|---|---|
| Treatment | (±SEM, mm^{∧}3) | TGI (%) | Isotype Control |
| PBS | 475.95 ± 43.76 | | |
| Isotype Control 10 mg/kg | 694.44 ± 187.73 | / | / |
| 18B10-HaLa low fucose 10 mg/kg | 152.22 ± 30.23 | 78.08 | 0.030 |
| IMAB362 10 mg/kg | 205.36 ± 60.55 | 70.43 | 0.0544 |

2. Efficacy on BxPC-3-CLDN18.2 Xenograft Model Using Nude Mice

BxPC-3-CLDN18.2 xenograft model was established and treated by antibodies following the same procedure of MIA PaCa-2-CLDN18.2 model (Section 1 of Example 19).

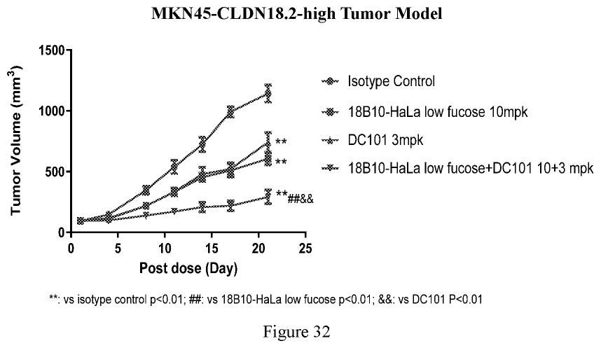

As shown in FIG. 36, IMAB362 could not inhibit tumor growth at all while 18B10-HaLa low fucose showed some degree of anti-tumor activity (Table 22). No significant body weight change was seen in all groups.

TABLE 22
Tumor Growth Inhibition (TGI) of antibodies in BxPC-3-CLDN18.2 Xenograft Model on
Day 34 (mean ± S.E.M., n = 6)

| | Tumor size | | p value vs. |
|---|---|---|---|
| Treatment | (±SEM, mm^{∧}3) | TGI (%) | Isotype Control |
| PBS | 801.64 ± 259.96 | | |
| Isotype Control 10 mg/kg | 902.02 ± 181.20 | / | / |
| 18B10-HaLa low fucose 10 mg/kg | 621.69 ± 157.93 | 31.08 | 0.3363 |
| IMAB362 10 mg/kg | 988.91 ± 169.91 | −9.63 | 0.7681 |

### Example 20: Anti-Tumor Activity of 18B10-HaLa Low Fucose on Lung Cancer Cells In Vitro

1. FACS Binding to Lung Cancer Cell Lines Using 18B10-HaLa Low Fucose

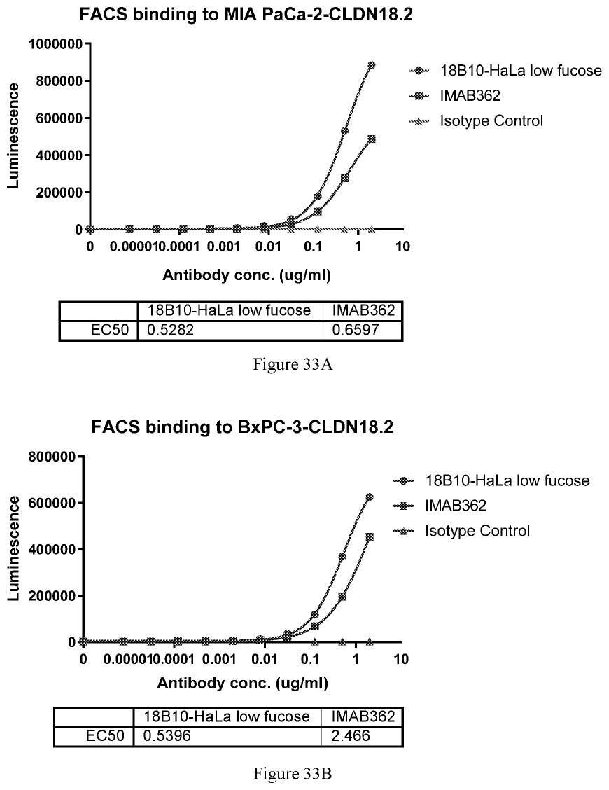

NCI-H146 was purchased from ATCC (Cat #, ATCC® HTB-173). NCI-H460-CLDN18.2 was purchase from Kyinno (Cat #, KC-1450), which was stable transfected with CLDN18.2. FACS binding was carried out following the same protocol of Section 2 of Example 7. As shown in FIG. 37A-37B, 18B10-HaLa low fucose could bind to the two cell lines in a dose-dependent manner. For much higher expression level of CLDN18.2 on NCI-H460-CLDN18.2 cell than NCI-H146 cell, the maximum binding signal of the former cell was also significantly higher than that of later one.

2. ADCC Reporter Assay on NCI-H146 Using Jurkat-NFAT-Luc-FcγRIIIA-V176 as the Effector Cells

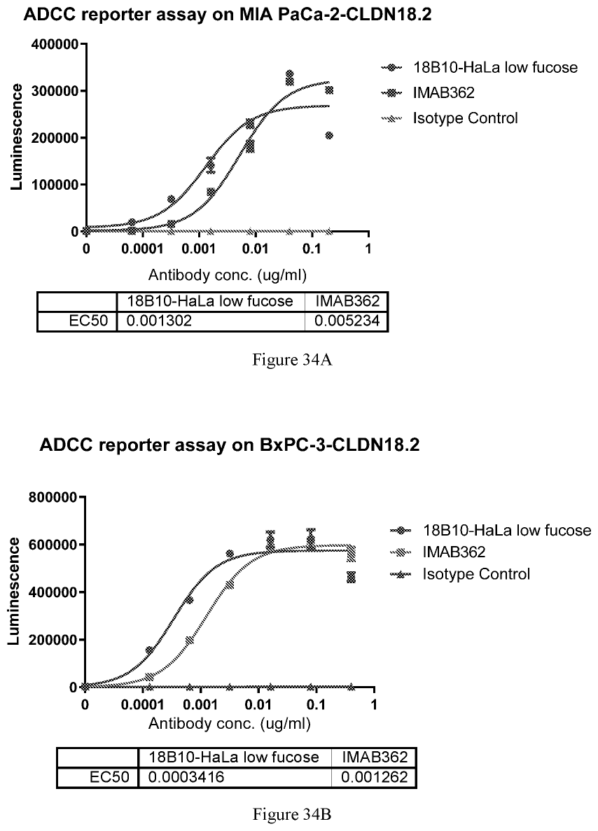

ADCC test was carried out following the same protocol above (see Section 2 of Example 7). As shown in FIG. 38, 18B10-HaLa low fucose could induce ADCC on NCI-H146 cell and the EC50 was around 0.003 μg/ml. Comparing to IMAB362, 18B10-HaLa low fucose had about 150-fold higher ADCC potency.

3. ADCC Assay on NCI-H460-CLDN18.2 Using PBMC as the Effector Cells

Primary PBMC mediated ADCC test was carried out following the similar protocol above (Section 6 of Example 16). Briefly, recover the frozen PBMC from liquid nitrogen and resuspend cells with RPMI1640+10% FBS at density of 5×10\hat{ }6/ml and incubate them in a 37° C. 5% CO2 incubator for 5 h before use. Label the target cell NCI-H460-CLDN18.2 cells or NCI-H292 cells with CellTrace™ Far Red following the instruction. Add the labeled target cells and diluted antibody into 24-well cell culture plate and incubate them in a 37° C. 5% CO2 incubator for 30 minutes. Then add PBMC cells into corresponding wells with E:T ratio of 40:1 and incubate cells in the incubator for 15 hours. At the end of culture, collect the suspension cells and adherent cells (with mild trypsin digestion) of each well into the corresponding 15 mL tube. Centrifuge the tubes to remove supernatant. Add PBS with Propidium Iodide (PI) Staining Solution to resuspend target cells and mark dead target cells. Analyze PI positive cell percentage in CellTrace™ Far Red positive cells by flow cytometry. Specific cytotoxicity was calculated by subtracting non-specific killing percentage.

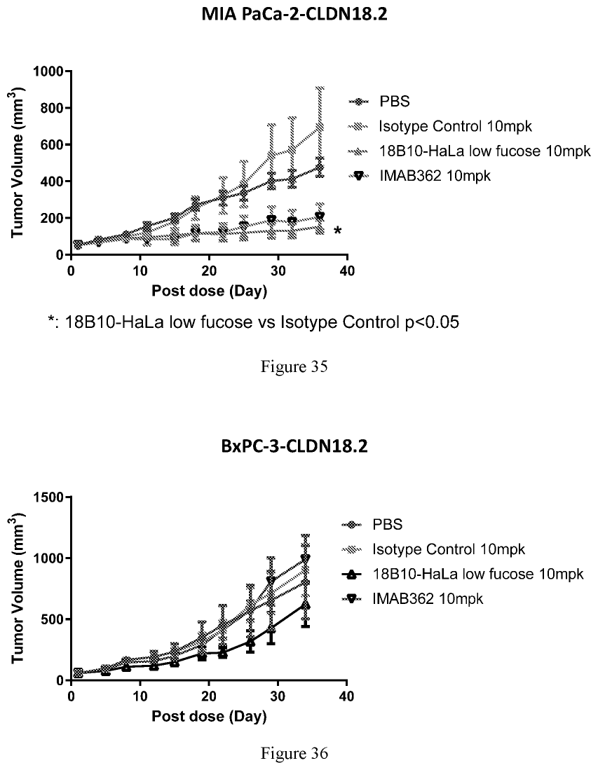

As shown in FIG. 39, 18B10-HaLa low fucose had ADCC activity only on NCI-H460-CLDN18.2 cell rather than NCI-H292, which is CLDN18.2 negative human lung adenocarcinoma cell.

### Example 21: Anti-Tumor Activity of 18B10-HaLa Low Fucose on Lung Cancer Cells In Vivo

1. Efficacy on NCI-H146 and Human PBMC Co-Inoculation Tumor Model Using Nude Mice

NCI-H146 and human PBMC co-inoculation tumor model was established and treated by antibodies following the same procedure of MKN45-CLDN18.2-high model (Section 1 of Example 17). 30 NOD-SCID mice were inoculated with 5×10\hat{ }6 NCI-H146+1.5×10\hat{ }6 human PBMC and 50% matri-gel. 4 hours after inoculation, animals were randomized to 3 μgroups (n=10).

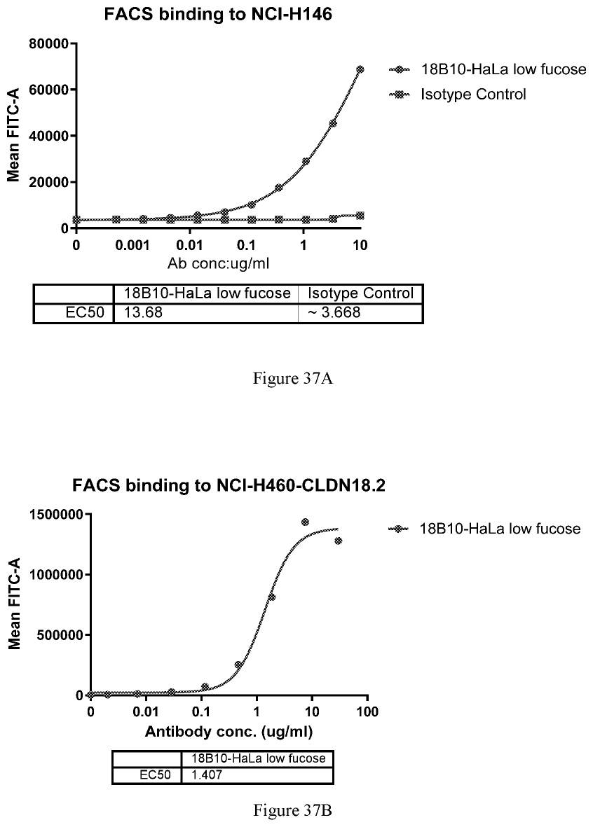

As shown in FIGS. 40A-40B and Table 23, with human PBMC and 18B10-HaLa low fucose treatment, NCI-H146 tumors almost didn't grow at all and there was very small intra group difference.

TABLE 23
Tumor Growth Inhibition (TGI) of antibodies in NCI-H146 and PBMC co-inoculation Model
on Day 32 (mean ± S.E.M., n = 10)

| | Tumor size | | p value vs. |
|---|---|---|---|
| Treatment | (±SEM, mm^{∧}3) | TGI (%) | Isotype Control |
| Model Group | 329.42 ± 70.14 | | |
| Isotype Control 10 mg/kg | 275.13 ± 55.10 | / | / |
| 18B10-HaLa low fucose 10 mg/kg | 85.91 ± 5.15 | 68.78 | 0.0001 |

2. Efficacy on NCI-H460-CLDN18.2 and Human PBMC Co-Inoculation Tumor Model Using NOD-SCID Mice

Human PBMC cells were acquired from Allcells. 20 female SPF grade NOD-SCID mice were randomized to 2 μgroup (n=10). Mice were inoculated with 3×10\hat{ }6 NCI-H460-CLDN18.2 cells and 5×10\hat{ }6 human PBMC cell with 50% matri-gel (BD) by s.c. injection on the right flank as model group. 4 hours after inoculation the mice were treated with 10 mg/kg isotype control and 10 mg/kg 18B10-HaLa low fucose, twice a week for 5 weeks by i.p. injection. Animals were sacrificed at the end of the study with C02 inhalation. Tumor size and volume were measured 2-3 times a week. Results were analyzed using Prism GraphPad and expressed as mean±S.E.M.

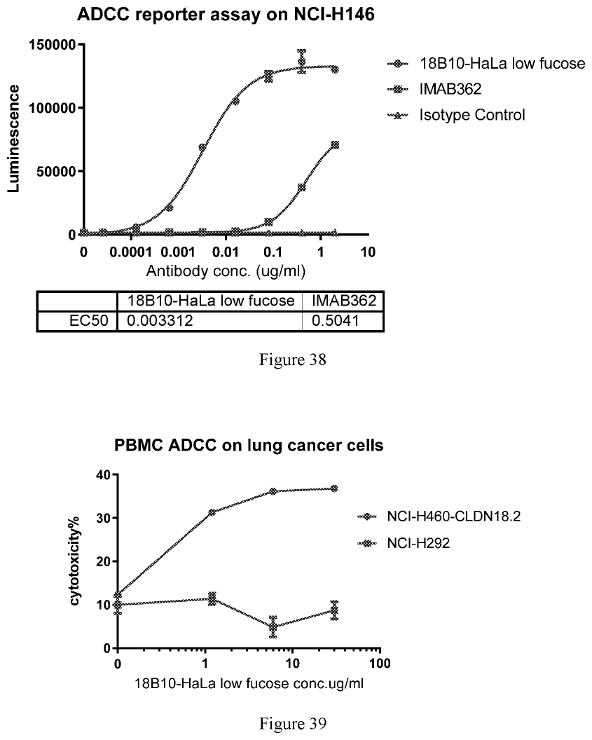

As shown in FIG. 41 and Table 24, with human PBMC, the tumor growth of 18B10-HaLa low fucose group was slower than that of isotype control group, with 36% TGI.

TABLE 24
Tumor Growth Inhibition (TGI) of antibodies in NCI-H460-CLDN18.2 tumor model on
Day 25 (mean ± S.E.M., n = 10)

| | Tumor size | | p value vs. |
|---|---|---|---|
| Treatment | (±SEM, mm^{∧}3) | TGI (%) | Isotype Control |
| Isotype Control 10 mg/kg | 486.54 ± 52.63 | | |
| 18B10-HaLa low fucose 10 mg/kg | 311.35 ± 32.78 | 36.01 | 0.0125 |

### Example 22: Anti-Tumor Activity of 18B10-HaLa Low Fucose on Colon Cancer Cells In Vitro

1. FACS Binding to Colon Cancer Cell Lines Using 18B10-HaLa Low Fucose

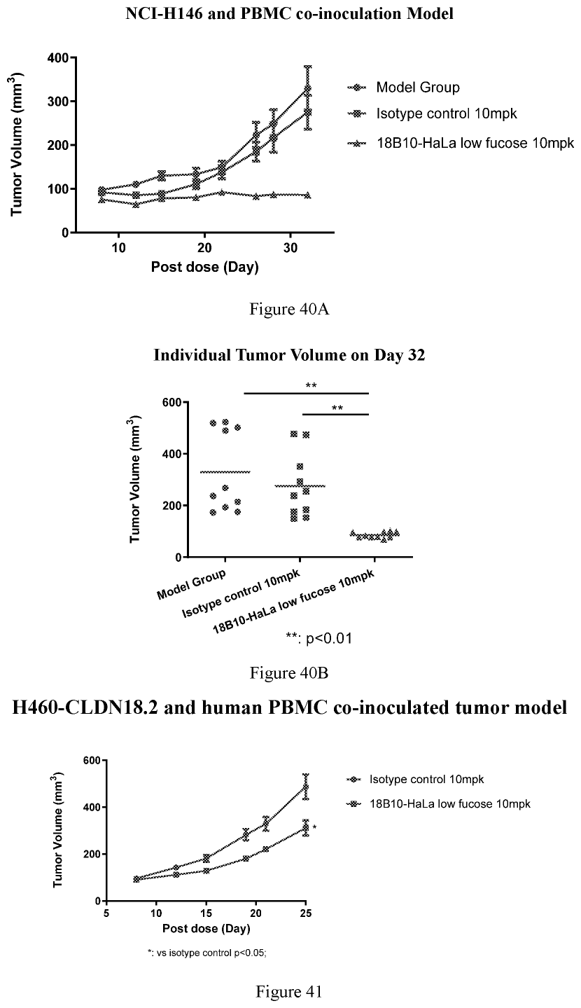

SK-CO-1 was purchased from ATCC (Cat #, ATCC® HTB-39). FACS binding was carried out following the same protocol of Section 2 of Example 7. As shown in FIG. 42, 18B10-HaLa low fucose could bind to SK-CO-1 cell in a dose-dependent manner. The EC50 was about 1.78 μg/ml.

2. ADCC Reporter Assay on Colon Cancer Cell Lines Using Jurkat-NFAT-Luc-FcγRIIIA-V176 as the Effector Cells

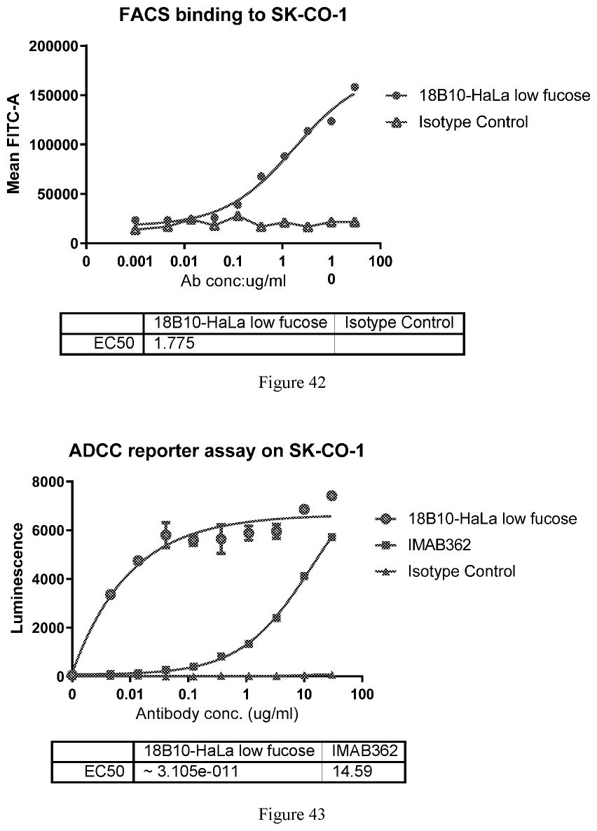

ADCC test was carried out following the same protocol above (see Section 2 of Example 7). As shown in FIG. 43, 18B10-HaLa low fucose could induce ADCC on SK-CO-1 cell. Comparing to IMAB362, 18B10-HaLa low fucose had much higher ADCC potency.

### Example 23: Interaction Analysis of 18B10-HaLa Low Fucose with Human Fc Receptors Using ForteBio Octet RED96

1. Interaction with FcγRIIIa Proteins

Biotinylated human FcγRIIIa-V176 or biotinylated human FcγRIIIa-F176 Protein with His tag at 100 nM in 1× Kinetics Buffer (1×PBS, pH 7.4, 0.002% Tween 20) were loaded onto 7 pre-wet SA biosensors (PALL, ForteBio, Cat #18-5019) and incubated with varying concentrations of 18B10-HaLa low fucose or IMAB362 in solution. All binding data were collected at 30° C. The experiments comprised 5 steps: 1. Baseline acquisition (60 s); 2. Biotinylated human FcγRIIIa-V176 Protein or biotinylated human FcγRIIIa-F176 Protein loading onto SA biosensor (120 s); 3. Second baseline acquisition (60 s); 4. Association of 18B10-HaLa low fucose or IMAB362 for the measurement of kₒₙ (60 s); and 5. Dissociation of antibodies for the measurement of koff (60 s). 7 different concentrations of antibodies were used, including 1000 nM, 500 nM, 250 nM, 125 nM, 62.5 nM, 31.3 nM and 0 nM, and the antibodies were diluted with 1× Kinetics Buffer. Baseline and dissociation steps were carried out in 1× Kinetics Buffer only. The ratio of koff to kₒₙ determines the KD. The Biosensors are regenerated for 5 s in Regeneration Buffer (10 mM Glycine-HCL, pH 1.5), followed by neutralization for 5 s in Neutralization Buffer (1×PBS, pH 7.4, 0.002% Tween 20), this process repeat 3 times.

As shown in Table 25, the affinities of 18B10-HaLa low fucose to human FcγRIIIa-V176 and human FcγRIIIa-F176 protein were a little higher than those of IMAB362, which may be caused by lower fucosylation.

TABLE 25
Kinetic binding constant of CLDN18.2 antibodies to human FcγRIIIa proteins

| Ligand | Analyte | kon (1/Ms) | kdis (1/s) | K_{D} (M) |
|---|---|---|---|---|
| FcγRIIIa-V176 | 18B10-HaLa low fucose | 3.59E+05 | 9.46E−03 | 2.64E−08 |
| | IMAB362 | 1.59E+05 | 9.75E−03 | 6.15E−08 |
| FcγRIIIa-F176 | 18B10-HaLa low fucose | 4.07E+05 | 3.88E−02 | 9.53E−08 |
| | IMAB362 | 1.42E+05 | 3.28E−02 | 2.32E−07 |

2. Interaction with FcRn (FCGRT&B2M) Protein

Human FcRn (FCGRT&B2M) Protein with His tag at 50 nM in FcRn 1×Kinetics Buffer (1×PBS, pH 6.0, 0.002% Tween 20) were loaded onto 7 pre-wet Ni-NTA biosensors and incubated with varying concentrations of 18B10-HaLa low fucose or IMAB362 in solution. All binding data were collected at 30° C. The experiments comprised 5 steps: 1. Baseline acquisition (60 s); 2. Human Fc gamma FcRn (FCGRT&B2M) Protein loading onto Ni-NTA biosensor (150 s); 3. Second baseline acquisition (80 s); 4. Association of 18B10-HaLa low fucose or IMAB362 for the measurement of kₒₙ (60 s); and 5. Dissociation of antibodies for the measurement of koff (60 s). 7 different concentrations of antibodies were used, including 500 nM, 250 nM, 125 nM, 62.5 nM, 31.3 nM, 15.6 nM and 0 nM, and the antibodies were diluted with FcRn Kinetics Buffer (pH 6.0). Baseline step was carried out in 1× Kinetics Buffer only, baseline2 and dissociation steps were carried out in FcRn Kinetics Buffer (pH 6.0). The ratio of koff to kₒₙ determines the KD. The Biosensors are regenerated for 5 s in Regeneration Buffer, followed by neutralization for 5 s in Neutralization Buffer, this process repeated 3 times.

As shown in Table 26, the affinity of 18B10-HaLa low fucose to human FcRn protein was not effected by lower fucosylation, even a little higher than that of IMAB362.

TABLE 26
Kinetic binding constant of CLDN18.2 antibodies to human FcRn protein

| Ligand | Analyte | kon (1/Ms) | kdis (1/s) | K_{D} (M) |
|---|---|---|---|---|
| FcRn | 18B10-HaLa low fucose | 6.59E+05 | 5.49E−03 | 8.34E−09 |
| (FCGRT&B2M) | IMAB362 | 4.46E+05 | 4.67E−03 | 1.05E−08 |

3. Interaction with Human C1q Protein

18B10-HaLa low fucose or IMAB362 were biotinylated by being mixed with Biotinamidohexanoic acid N-hydroxysuccinimide ester (Sigma) in DMF. Biotinylated 18B10-HaLa low fucose or IMAB362 at 100 nM in 1× Kinetics Buffer were loaded onto 7 pre-wet SA biosensors and incubated with varying concentrations of human C1q in solution. All binding data were collected at 30° C. The experiments comprised 5 steps: 1. Baseline acquisition (60 s); 2. Biotinylated 18B10-HaLa low fucose or IMAB362 loading onto SA biosensor (150 s); 3. Second baseline acquisition (60 s); 4. Association of human C1q for the measurement of kₒₙ (30 s); and 5. Dissociation of human C1q for the measurement of off (30 s). 7 different concentrations of antibodies were used, including 50 nM, 25 nM, 12.5 nM, 6.25 nM, 3.13 nM, 1.56 nM and 0 nM, and the human C1q was diluted with 1× Kinetics Buffer. Baseline and dissociation steps were carried out in 1× Kinetics Buffer only. The ratio of koff to kₒₙ determines the KD. The Biosensors are only used once.

As shown in Table 27, the affinity of 18B10-HaLa low fucose to human C1q protein was not quite similar to that of IMAB362.

TABLE 27
Kinetic binding constant of CLDN18.2 antibodies to human C1q protein

| Ligand | Analyte | kon (1/Ms) | kdis (1/s) | K_{D} (M) |
|---|---|---|---|---|
| 18B10-HaLa low fucose | C1q | 7.96E+06 | 1.37E−01 | 1.72E−08 |
| IMAB362 | | 6.87E+06 | 1.48E−01 | 2.15E−08 |

1. An isolated antibody against human CLDN18.2 or an antigen-binding fragment thereof, capable of binding to an epitope comprising at least one, two, or three of amino acid residues at positions D28, W30, V43, N45, Y46, L49, W50, R51, R55, E56, F60, E62, Y66, L72, L76, V79 and R80 in the amino acid sequence of SEQ ID NO: 30.

2. The isolated antibody or an antigen-binding fragment thereof of claim 1, wherein the epitope comprises the amino acid residue at position E56.

3. The isolated antibody or an antigen-binding fragment thereof of claim 1 or 2, wherein the epitope does not contain at least one of the following residues: A42, or N45.

4. The isolated antibody or an antigen-binding fragment thereof of any of preceding claims, wherein the epitope comprises the amino acid residue at position W30, L49, W50, R55, and E56.

5. The isolated antibody or an antigen-binding fragment thereof of any of preceding claims, wherein the epitope further comprises one or more amino acid residues: T41, N45, Y46, R51, F60, E62, and R80.

6. The isolated antibody or an antigen-binding fragment thereof of any of preceding claims, wherein the epitope further comprises one or more amino acid residues: D28, V43, N45, Y46, Y66, L72, L76, and V79.

7. An isolated antibody or an antigen-binding fragment thereof, capable of specifically binding to human CLDN18.2 and having at least one of the following characteristics:
a) binding to a cell expressing human CLDN18.2 at a Kd value of no more than 2.5 nM (or no more than 2.0, 1.5, 1, 0.9, 0.8, 0.7, 0.6, 0.5, 0.4 nM) as measured by KinExA assay;
b) binding to a cell expressing human CLDN18.2 at an EC50 value of no more than 70 μg/ml (or no more than 65, 60, 55, 50, 45, 40, 35, 30, 25, 20, 15, 12, or 10, 9, 8, 7, 6, 5, 4, 3, 2, or 1 μg/ml) as measured by flow cytometry;
c) inducing complement dependent cytotoxicity (CDC) on a cell expressing human CLDN18.2 at an EC50 value of no more than 1 μg/ml (or no more than 0.9, 0.8, 0.7, 0.6, 0.5 μg/ml) as measured by cytotoxicity assay,
d) inducing antibody-dependent cell cytotoxicity (ADCC) on a cell expressing human CLDN18.2 at an EC50 value of no more than 2 μg/ml (or no more than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 μg/ml) as measured by an ADCC reporter assay.

8. The isolated antibody or an antigen-binding fragment thereof of claim 7, wherein the cell comprises NUGC4 cell, SNU-620 cell, SNU-601 cell, KATOIII cell, or a comparable cell thereof having a human CLDN18.2 protein expression level comparable to or no more than that of NUGC4 cell, SNU-620 cell, SNU-601 cell, or KATOIII cell.

9. The isolated antibody or an antigen-binding fragment thereof of claim 7, wherein the cell comprises a human CLDN18.2 high-expressing cell, a human CLDN18.2 medium-expressing cell, or a human CLDN18.2 low-expressing cell, wherein the human CLDN18.2 high-expressing cell expresses human CLDN18.2 at an intensity of at least 2+ as measured by IHC and at a level where at least 40% of the cells are stained positive in Immunohistochemistry (IHC); the human CLDN18.2 medium-expressing cell expresses human CLDN18.2 at an intensity of at least 1+ and below 2+ as measured by IHC and at a level where at least 30% but below 40% of the cells are stained positive in IHC; and the human CLDN18.2 low-expressing cell expresses human CLDN18.2 at an intensity of above 0 but below 1+ as measured by IHC and at a level where above 0 but below 30% of the cells are stained positive in IHC.

10. The isolated antibody or an antigen-binding fragment thereof of claim 7, wherein the EC50 value for binding to NUGC4 cells is no more than 70 μg/ml (or no more than 65, 60, 55, 50, 45, 40, 35, 30, 25, 20, 15, 12, or 10 μg/ml).

11. The isolated antibody or an antigen-binding fragment thereof of claim 7, wherein the ADCC on NUGC4 cells at an EC50 value of no more than 2 μg/ml (or no more than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 μg/ml) as measured by an ADCC reporter assay.

12. An isolated antibody or an antigen-binding fragment thereof, capable of specifically binding to human CLDN18.2 and having at least one of the following characteristics:
a) binding to human CLDN18.2 at an Kd value no more than 80%, 70%, 60%, 50%, 40%, 30%, 20%, 15% of that of IMAB362, as measured by KinExA assay;
b) binding to a cell expressing human or mouse CLDN18.2 at an EC50 value no more than 80%, 70%, 60%, 50%, 40%, 30%, 20%, 15% or 10% of that of IMAB362, as measured by flow cytometry assay;
c) inducing complement dependent cytotoxicity (CDC) on a cell expressing human CLDN18.2 at an EC50 value no more than 80%, 70%, 60%, 50%, 40%, 30%, 20%, 10%, or 5% of than that of IMAB362, as measured by cytotoxicity assay; and
d) inducing antibody-dependent cell cytotoxicity (ADCC) on a cell expressing human CLDN18.2 at an EC50 value no more than 80%, 70%, 60%, 50%, 40%, 30%, 20%, 10%, 5%, or 1% of that of IMAB362, as measured by an ADCC reporter assay,
wherein IMAB362 is an antibody comprising a heavy chain variable region comprising the amino acid sequence of SEQ ID NO: 72, and a light chain variable region comprising the amino acid sequence of SEQ ID NO: 73.

13. The isolated antibody or an antigen-binding fragment thereof of claim 12, wherein the cell comprises a NUGC4 cell, SNU-620 cell, SNU-601 cell, KATOIII cell, or a cell line having a human CLDN18.2 protein expression level comparable to or no more than that of NUGC4 cell, SNU-620 cell, SNU-601 cell, KATOIII cell.

14. The isolated antibody or an antigen-binding fragment thereof of claim 12, wherein the cell comprises a human CLDN18.2 high-expressing cell, a human CLDN18.2 medium-expressing cell, or a human CLDN18.2 low-expressing cell, wherein the human CLDN18.2 high-expressing cell expresses human CLDN18.2 at an intensity of at least 2+ as measured by IHC and at a level where at least 40% of the cells are stained positive in IHC; the human CLDN18.2 medium-expressing cell expresses human CLDN18.2 at an intensity of at least 1+ and below 2+ as measured by IHC and at a level where at least 30% but below 40% of the cells are stained positive in IHC; and the human CLDN18.2 low-expressing cell expresses human CLDN18.2 at an intensity of above 0 but below 1+ as measured by IHC and at a level where above 0 but below 30% of the cells are stained positive in IHC.

15. The isolated antibody or an antigen-binding fragment thereof of any of claims 7-14, wherein the antibody binds to an epitope comprising at least one, two, or three of amino acid residues at positions D28, W30, V43, N45, Y46, L49, W50, R51, R55, E56, F60, E62, Y66, L72, L76, V79 and R80 in the amino acid sequence of SEQ ID NO: 30, optionally, the epitope comprises the amino acid residue at position E56; optionally, the epitope does not contain at least one of the following residues: A42, or N45; optionally, the epitope comprises the amino acid residue at position W30, L49, W50, R55, and E56; optionally, the epitope further comprises one or more amino acid residues: T41, N45, Y46, R51, F60, E62, and R80; and optionally, the epitope further comprises one or more amino acid residues: D28, V43, N45, Y46, Y66, L72, L76, and V79.

16. An anti-CLDN18.2 antibody or an antigen-binding fragment thereof, comprising heavy chain HCDR1, HCDR2 and HCDR3 and/or light chain LCDR1, LCDR2 and LCDR3 sequences, wherein:
the HCDR1 sequence comprises GYNMN (SEQ ID NO: 1), or TYFIGVG (SEQ ID NO: 13), or a homologue sequence of at least 80% sequence identity thereof;
the HCDR2 sequence comprises X₁IDPYYX₂X₃TX₄YNQKFX₅G (SEQ ID NO: 32), or HIWWNDNKYYNTALKS (SEQ ID NO: 15), or a homologue sequence of at least 80% sequence identity thereof;
the HCDR3 sequence comprises X₆X₇X₈GNAFDY (SEQ ID NO: 33), or MGSGAWFTY (SEQ ID NO: 17), or a homologue sequence of at least 80% sequence identity thereof;
the LCDR1 sequence comprises KSSQX₉LX₁₀NX₁₁GNX₁₂KNYLT (SEQ ID NO: 34) or a homologue sequence of at least 80% sequence identity thereof;
the LCDR2 sequence comprises WASTRX₁₃S (SEQ ID NO: 35) or a homologue sequence of at least 80% sequence identity thereof;
the LCDR3 sequence comprises QNDYX₁₄X₁₅PX₁₆T (SEQ ID NO: 36) or a homologue sequence of at least 80% sequence identity thereof,
wherein X₁ is N or Y or H, X₂ is G or V, X₃ is A or G or T, X₄ is R or T or S, X₅ is K or R, X₆ is S or M, X₇ is Y or F, X₈ is Y or H, X₉ is S or N, X₁₀ is L or F, X₁₁ is S or N, X₁₂ is Q or L, X₁₃ is E or K, X₁₄ is S or Y, X₁₅ is F or Y and X₁₆ is F or L.

17. An anti-CLDN18.2 antibody or an antigen-binding fragment thereof, wherein the heavy chain variable region comprises:
a) a HCDR1 comprises a sequence selected from SEQ ID NO: 1, and SEQ ID NO: 13,
b) a HCDR2 comprises a sequence selected from SEQ ID NO: 3, SEQ ID NO: 7, SEQ ID NO: 9, SEQ ID NO: 15, SEQ ID NO: 19, and SEQ ID NO: 22, and
c) a HCDR3 comprises a sequence selected from SEQ ID NO: 5, SEQ ID NO: 11, SEQ ID NO: 17, and SEQ ID NO: 21, and/or
a light chain variable region comprising:
d) a LCDR1 comprises a sequence of SEQ ID NO: 2, SEQ ID NO: 10, SEQ ID NO: 14, and SEQ ID NO: 20,
e) a LCDR2 comprises a sequence of SEQ ID NO: 4, and SEQ ID NO: 16, and
f) a LCDR3 comprises a sequence selected from SEQ ID NO: 6, SEQ ID NO: 8, SEQ ID NO: 12, and SEQ ID NO: 18.

18. The antibody or an antigen-binding fragment thereof of any of the preceding claims, wherein the heavy chain variable region is selected from the group consisting of:
a heavy chain variable region comprising a HCDR1 comprising the sequence of SEQ ID NO: 1, a HCDR2 comprising the sequence of SEQ ID NO: 3, and a HCDR3 comprising the sequence of SEQ ID NO: 5;
a heavy chain variable region comprising a HCDR1 comprising the sequence of SEQ ID NO: 1, a HCDR2 comprising the sequence of SEQ ID NO: 7, and a HCDR3 comprising the sequence of SEQ ID NO: 5;
a heavy chain variable region comprising a HCDR1 comprising the sequence of SEQ ID NO: 1, a HCDR2 comprising the sequence of SEQ ID NO: 9, and a HCDR3 comprising the sequence of SEQ ID NO: 11;
a heavy chain variable region comprising a HCDR1 comprising the sequence of SEQ ID NO: 13, a HCDR2 comprising the sequence of SEQ ID NO: 15, and a HCDR3 comprising the sequence of SEQ ID NO: 17;
a heavy chain variable region comprising a HCDR1 comprising the sequence of SEQ ID NO: 1, a HCDR2 comprising the sequence of SEQ ID NO: 19, and a HCDR3 comprising the sequence of SEQ ID NO: 21; and
a heavy chain variable region comprising a HCDR1 comprising the sequence of SEQ ID NO: 1, a HCDR2 comprising the sequence of SEQ ID NO: 22, and a HCDR3 comprising the sequence of SEQ ID NO: 5.

19. The antibody or an antigen-binding fragment thereof of any of the preceding claims, wherein the light chain variable region is selected from the group consisting of:
a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 6;
a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 8;
a light chain variable region comprising a LCDR1 comprising the sequence of SEQ ID NO: 10, a LCDR2 comprising the sequence of SEQ ID NO: 4, and a LCDR3 comprising the sequence of SEQ ID NO: 6;
a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 12;
a light chain variable region comprising a LCDR1 comprising the sequence of SEQ ID NO: 14, a LCDR2 comprising the sequence of SEQ ID NO: 16, and a LCDR3 comprising the sequence of SEQ ID NO: 18; and
a light chain variable region comprising a LCDR1 comprising the sequence of SEQ ID NO: 20, a LCDR2 comprising the sequence of SEQ ID NO: 4, and a LCDR3 comprising the sequence of SEQ ID NO: 6.

20. The antibody or an antigen-binding fragment thereof of any of the preceding claims, wherein:
the heavy chain variable region comprises a HCDR1 comprising the sequence of SEQ ID NO: 1, a HCDR2 comprising the sequence of SEQ ID NO: 3, and a HCDR3 comprising the sequence of SEQ ID NO: 5; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 6;
the heavy chain variable region comprises a HCDR1 comprising the sequence of SEQ ID NO: 1, a HCDR2 comprising the sequence of SEQ ID NO: 7, and a HCDR3 comprising the sequence of SEQ ID NO: 5; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 8;
the heavy chain variable region comprises a HCDR1 comprising the sequence of SEQ ID NO: 1, a HCDR2 comprising the sequence of SEQ ID NO: 9, and a HCDR3 comprising the sequence of SEQ ID NO: 11; and the light chain variable region comprises a LCDR1 comprising the sequence of SEQ ID NO: 10, a LCDR2 comprising the sequence of SEQ ID NO: 4, and a LCDR3 comprising the sequence of SEQ ID NO: 6;
the heavy chain variable region comprises a HCDR1 comprising the sequence of SEQ ID NO: 13, a HCDR2 comprising the sequence of SEQ ID NO: 15, and a HCDR3 comprising the sequence of SEQ ID NO: 17; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 12;
the heavy chain variable region comprises a HCDR1 comprising the sequence of SEQ ID NO: 1, a HCDR2 comprising the sequence of SEQ ID NO: 19, and a HCDR3 comprising the sequence of SEQ ID NO: 21; and the light chain variable region comprises a LCDR1 comprising the sequence of SEQ ID NO: 14, a LCDR2 comprising the sequence of SEQ ID NO: 16, and a LCDR3 comprising the sequence of SEQ ID NO: 18; or
the heavy chain variable region comprises a HCDR1 comprising the sequence of SEQ ID NO: 1, a HCDR2 comprising the sequence of SEQ ID NO: 22, and a HCDR3 comprising the sequence of SEQ ID NO: 5; and the light chain variable region comprises a LCDR1 comprising the sequence of SEQ ID NO: 20, a LCDR2 comprising the sequence of SEQ ID NO: 4, and a LCDR3 comprising the sequence of SEQ ID NO: 6.

21. The antibody or an antigen-binding fragment thereof of any one of the preceding claims, further comprising one or more of heavy chain HFR1, HFR2, HFR3 and HFR4, and/or one or more of light chain LFR1, LFR2, LFR3 and LFR4, wherein:
the HFR1 comprises QVQLVQSGAEVKKPGASVKVSCKASGYX₁₇FT (SEQ ID NO: 54) or a homologous sequence of at least 80% sequence identity thereof,
the HFR2 comprises WVX₁₈QAPGQGLEWX₁₉G (SEQ ID NO: 55) or a homologous sequence of at least 80% sequence identity thereof,
the HFR3 sequence comprises RVTX₂₀TIDKSTSTVYMELSSLRSEDTAVYYCAR (SEQ ID NO: 56) or a homologous sequence of at least 80% sequence identity thereof,
the HFR4 comprises WGQGTTVTVSS (SEQ ID NO: 57) or a homologous sequence of at least 80% sequence identity thereof,
the LFR1 comprises DIVMTQSPDSLAVSLGERATX₂₁NC (SEQ ID NO: 58) or a homologous sequence of at least 80% sequence identity thereof,
the LFR2 comprises WYQQKPGQPPKLLIY (SEQ ID NO: 59) or a homologous sequence of at least 80% sequence identity thereof,
the LFR3 comprises GVPDRFX₂₂GSGSGTDFTLTISSLQAEDVAVYYC (SEQ ID NO: 60) or a homologous sequence of at least 80% sequence identity thereof, and
the LFR4 comprises FGGGTKVEIK (SEQ ID NO: 61) or a homologous sequence of at least 80% sequence identity thereof,
wherein X₁₇ is T or S, X₁₈ is R or K, X₁₉ is M or I, X₂₀ is M or L, X₂₁ is I or M, and X₂₂ is S or T.

22. The antibody or antigen-binding fragment thereof of claim 21, wherein:
the HFR1 comprises a sequence selected from the group consisting of SEQ ID NOs: 62 and 63,
the HFR2 comprises a sequence selected from the group consisting of SEQ ID NOs: 64 and 65,
the HFR3 comprises the sequence selected from the group consisting of SEQ ID NOs: 66 and 67,
the HFR4 comprises a sequence of SEQ ID NOs: 57,
the LFR1 comprises the sequence from the group consisting of SEQ ID NOs: 68 and 69,
the LFR2 comprises a sequence of SEQ ID NO: 59,
the LFR3 comprises a sequence selected from the group consisting of SEQ ID NOs: 70 and 71, and
the LFR4 comprises a sequence of SEQ ID NO: 61.

23. The antibody or an antigen-binding fragment thereof of any of the preceding claims, wherein the heavy chain variable region comprises a sequence selected from the group consisting of SEQ ID NO: 25, SEQ ID NO: 27, SEQ ID NO: 29, SEQ ID NO: 37, SEQ ID NO: 39, SEQ ID NO: 41, SEQ ID NO: 43, SEQ ID NO: 45, and SEQ ID NO: 47, and a homologous sequence thereof having at least 80% sequence identity yet retaining specific binding affinity to CLDN18.2.

24. The antibody or an antigen-binding fragment thereof of any of the preceding claims, wherein the light chain variable region comprises a sequence selected from the group consisting of SEQ ID NO: 26, SEQ ID NO: 28, SEQ ID NO: 38, SEQ ID NO: 40, SEQ ID NO: 42, SEQ ID NO: 44, SEQ ID NO: 46, SEQ ID NO: 48, and a homologous sequence thereof having at least 80% sequence identity yet retaining specific binding affinity to CLDN18.2.

25. The antibody or an antigen-binding fragment thereof of any of the preceding claims, wherein:
a heavy chain variable region comprising the sequence of SEQ ID NO: 23 and a light chain variable region comprising the sequence of SEQ ID NO: 24;
the heavy chain variable region comprises a sequence of SEQ ID NO: 25 and the light chain variable region comprises a sequence of SEQ ID NO: 26;
the heavy chain variable region comprises a sequence of SEQ ID NO: 27 and the light chain variable region comprises a sequence of SEQ ID NO: 28;
the heavy chain variable region comprises a sequence of SEQ ID NO: 29 and the light chain variable region comprises a sequence of SEQ ID NO: 26, or 28;
the heavy chain variable region comprises a sequence of SEQ ID NO: 37 and the light chain variable region comprises a sequence of SEQ ID NO: 38;
the heavy chain variable region comprises a sequence of SEQ ID NO: 39 and the light chain variable region comprises a sequence of SEQ ID NO: 40;
the heavy chain variable region comprises a sequence of SEQ ID NO: 41 and the light chain variable region comprises a sequence of SEQ ID NO: 42;
the heavy chain variable region comprises a sequence of SEQ ID NO: 43 and the light chain variable region comprises a sequence of SEQ ID NO: 44;
the heavy chain variable region comprises a sequence of SEQ ID NO: 45 and the light chain variable region comprises a sequence of SEQ ID NO: 46; or
the heavy chain variable region comprises a sequence of SEQ ID NO: 47 and the light chain variable region comprises a sequence of SEQ ID NO: 48.

26. The antibody or antigen-binding fragment thereof of any of the preceding claims, further comprising one or more amino acid residue substitutions or modifications yet retains specific binding affinity to human CLDN18.2.

27. The antibody or antigen-binding fragment thereof of claim 26, wherein at least one of the substitutions or modifications is in one or more of the CDR sequences, and/or in one or more of the non-CDR regions of the VH or VL sequences.

28. The antibody or an antigen-binding fragment thereof of any of claims 16-27, wherein the antibody binds to an epitope comprising at least one, two, or three of amino acid residues at positions D28, W30, V43, N45, Y46, L49, W50, R51, R55, E56, F60, E62, Y66, L72, L76, V79 and R80 of human CLDN18.2 having the amino acid sequence of SEQ ID NO: 30.

29. The antibody or antigen-binding fragment thereof of any of the preceding claims, further comprising an immunoglobulin constant region, optionally a constant region of human Ig, or optionally a constant region of human IgG.

30. The antibody or antigen-binding fragment thereof of claim 29, wherein the constant region comprises a constant region of human IgG1, IgG2, IgG3, or IgG4.

31. The antibody or antigen-binding fragment thereof of claim 30, wherein the constant region of human IgG1 comprises SEQ ID NO: 49, or a homologous sequence having at least 80% sequence identity thereof.

32. The antibody or antigen-binding fragment thereof of any of claims 29-31, wherein the constant region comprises one or more amino acid residue substitutions or modifications conferring increased CDC or ADCC relative to wild-type constant region.

33. The antibody or antigen-binding fragment thereof of claim 32, wherein the constant region comprises one or more amino acid residue substitutions relative to SEQ ID NO: 49, selected from the group consisting of: L235V, F243L, R292P, Y300L, P396L, or any combination thereof.

34. The antibody or antigen-binding fragment thereof of claim 33, wherein the constant region comprises the sequence of SEQ ID NO: 51.

35. The antibody or an antigen-binding fragment thereof of any of the preceding claims, which is humanized.

36. The antibody or antigen-binding fragment thereof of any of the preceding claims, which is afucosylated.

37. The antibody or antigen-binding fragment thereof of any of the preceding claims, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

38. The antibody or antigen-binding fragment thereof of any of the preceding claims, which is bispecific.

39. The antibody or antigen-binding fragment thereof of claim 38, capable of specifically binding to a first and a second epitope of CLDN18.2, or capable of specifically binding to CLDN18.2 and a second antigen.

40. The antibody or antigen-binding fragment thereof of claim 39, wherein the second antigen is an immune related target, optionally selected from the group consisting of: PD-L1, PD-L2, PD-1, CLTA-4, TIM-3, LAG3, CD160, 2B4, TGF 3, VISTA, BTLA, TIGIT, LAIR1, OX40, CD2, CD27, ICAM-1, NKG2C, SLAMF7, NKp80, CD160, B7-H3, LFA-1, 1COS, 4-1BB, GITR, CD30, CD40, BAFFR, HVEM, CD7, LIGHT, IL-2, IL-15, CD3, CD16 and CD83.

41. The antibody or antigen-binding fragment thereof of claim 39, wherein the second antigen comprises a tumor antigen.

42. The antibody or antigen-binding fragment thereof of claim 41, wherein the tumor antigen is present in a CLDN18.2-expressing cell.

43. The antibody or antigen-binding fragment thereof of claim 42, wherein the tumor antigen comprises CA-125, gangliosides G (D2), G (M2) and G (D3), CD20, CD52, CD33, Ep-CAM, CEA, bombesin-like peptides, PSA, HER2/neu, epidermal growth factor receptor (EGFR), erbB2, erbB3/HER3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R or CO17-1A.

44. The antibody or an antigen-binding fragment thereof of any of the preceding claims, capable of specifically binding to mouse CLDN18.2.

45. The antibody or an antigen-binding fragment thereof of any of the preceding claims, which does not bind to human CLDN18.1.

46. The antibody or antigen-binding fragment thereof of any of the preceding claims linked to one or more conjugate moieties.

47. The antibody or antigen-binding fragment thereof of claim 46, wherein the conjugate moiety comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

48. An antibody or an antigen-binding fragment thereof, which competes for binding to CLDN18.2 with the antibody or antigen-binding fragment thereof of any of claims 1-6, and 16-47.

49. A composition comprising the anti-CLDN18.2 antibody or antigen-binding fragment thereof of any of claims 1-48, wherein the antibodies or antigen-binding fragments thereof is afucosylated.

50. The composition of claim 49, wherein the anti-CLDN18.2 antibody in the composition has an amount of fucose of 60% or less (e.g. less than 55%, 50%, 45%, 40%, 35%, 30%, 25%, 20%, 15% or 10%) of the total amount of oligosaccharides at Asn297 according to the EU numbering system.

51. A pharmaceutical composition comprising the antibody or antigen-binding fragment thereof of any of the preceding claims, and one or more pharmaceutically acceptable carriers.

52. An isolated polynucleotide encoding the antibody or an antigen-binding fragment thereof of the preceding claims.

53. A vector comprising the isolated polynucleotide of claim 52.

54. A host cell comprising the vector of claim 53.

55. A method of expressing the antibody or antigen-binding fragment thereof of any of claims 1-48, comprising culturing the host cell of claim 54 under the condition at which the vector of claim 53 is expressed.

56. A method of treating a disease or condition in a subject that would benefit from modulation of CLDN18.2 activity, comprising administering to the subject a therapeutically effective amount of the antibody or antigen-binding fragment thereof of any of claims 1-48 and/or the pharmaceutical composition of claim 51.

57. The method of claim 56, wherein the disease or condition is a CLDN18.2 related disease or condition.

58. The method of claim 57, wherein the disease or condition is cancer, optionally CLDN18.2-expressing cancer.

59. The method of any of claims 56-58, wherein the subject is identified as having a CLDN18.2-expressing cancer cell.

60. The method of claim 59, wherein the subject is identified as having a CLDN18.2 high-expressing cancer cell, a CLDN18.2 medium-expressing cancer cell, or a CLDN18.2 low-expressing cancer cell.

61. The method of claim 60, wherein the CLDN18.2 high-expressing cancer cell expresses CLDN18.2 at an intensity of at least 2+ as measured by IHC and at a level where at least 40% of the cells are stained positive in IHC; the CLDN18.2 medium-expressing cancer cell expresses CLDN18.2 at an intensity of at least 1+ and below 2+ as measured by IHC and at a level where at least 30% but below 40% of the cells are stained positive in IHC, and the CLDN18.2 low-expressing cancer cell expresses CLDN18.2 at an intensity of above 0 but below 1+ as measured by IHC and at a level where above 0 but below 30% of the cells are stained positive in IHC.

62. The method of any of claims 56-61, wherein the cancer is gastric cancer, lung cancer, bronchial cancer, bone cancer, liver and bile duct cancer, pancreatic cancer, breast cancer, liver cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, and adenocarcinoma.

63. The method of any of claims 56-62, wherein the subject is human.

64. The method of any of claims 56-63, wherein the administration is via oral, nasal, intravenous, subcutaneous, sublingual, or intramuscular administration.

65. The method of any of claims 56-64, further comprising administering a therapeutically effective amount of a second therapeutic agent.

66. The method of any of claim 65, wherein the second therapeutic agent is selected from a chemotherapeutic agent, an anti-cancer drug, radiation therapy, an immunotherapy agent, anti-angiogenesis agent, a targeted therapy agent, a cellular therapy agent, a gene therapy agent, a hormonal therapy agent, or cytokines.

67. A kit comprising an antibody or an antigen-binding fragment thereof of any of claims 1-48, and a second therapeutic agent.

68. A method of modulating CLDN18.2 activity in a CLDN18.2-expressing cell, comprising exposing the CLDN18.2-expressing cell to the antibody or antigen-binding fragment thereof of any of claims 1-48.

69. A method of detecting presence or amount of CLDN18.2 in a sample, comprising contacting the sample with the antibody or antigen-binding fragment thereof of any of claims 1-48, and determining the presence or the amount of CLDN18.2 in the sample.

70. A method of diagnosing a CLDN18.2 related disease or condition in a subject, comprising: a) contacting a sample obtained from the subject with the antibody or antigen-binding fragment thereof of any of claims 1-48; b) determining presence or amount of CLDN18.2 in the sample; and c) correlating the presence or the amount of CLDN18.2 to existence or status of the CLDN18.2 related disease or condition in the subject.

71. Use of the antibody or antigen-binding fragment thereof of any of claims 1-48 in the manufacture of a medicament for treating a CLDN18.2 related disease or condition in a subject.

72. Use of the antibody or antigen-binding fragment thereof of any of claims 1-48 in the manufacture of a diagnostic reagent for diagnosing a CLDN18.2 related disease or condition.

73. A kit comprising the antibody or antigen-binding fragment thereof of any of claims 1-48, useful in detecting CLDN18.2.

74. A chimeric antigen receptor (CAR) comprising an antigen binding domain, a transmembrane domain, a costimulatory signaling region, and a TCR signaling domain, wherein the antigen binding domain specifically binds to CLDN18.2 and comprises an antigen binding fragment of any of claims 1-48.

75. The CAR of claim 74, wherein the antigen binding fragment is a Fab or a scFv.

76. The CAR of claim 74 or 75, which is bispecific.

77. The CAR of claim 75, wherein the CAR is capable of further specifically binding to a second antigen other than CLDN18.2, or a second epitope on CLDN18.2.

78. The CAR of claim 77, wherein the second antigen comprises a tumor antigen.

79. A nucleic acid sequence encoding the chimeric antigen receptor (CAR) of any one of claims 74-78.

80. A cell comprising the nucleic acid sequence of claim 79.

81. A cell genetically modified to express the CAR of any one of claims 74-78.

82. A vector comprising the nucleic acid sequence of claim 79.

83. A method for stimulating a T cell-mediated immune response to a CLDN18.2-expressing cell or tissue in a mammal, the method comprising administering to the mammal an effective amount of a cell genetically modified to express the CAR of any one of claims 74-78.

84. A method of treating a mammal having a CLDN18.2 related disease or condition, comprising administering to the mammal an effective amount of a cell of claim 81, thereby treating the mammal.

85. The method of claim 84, wherein the cell is an autologous T cell.

86. The method of claim 84, wherein the CLDN18.2 related disease or condition is cancer.

87. The method of claim 84, wherein the mammal is a human subject.

88. The method claim 84, wherein the mammal is identified as having a CLDN18.2-expressing cancer cell, optionally the mammal is identified as having a CLDN18.2 high-expressing cancer cell, a CLDN18.2 medium-expressing cancer cell, or a CLDN18.2 low-expressing cancer cell.
